(19) United States
(12) Patent Application Publication
(10) Pub. No.: US 2026/0076551 A1
(43) Pub. Date: Mar. 19, 2026

(54) MACHINE-LEARNING-BASED CONTACT LENS FITTING ANALYSIS

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/885,484

(22) Filed: Sep. 13, 2024

(51) Int. Cl. A61B 3/04; A61B 3/00; A61B 3/06

(52) U.S. Cl. CPC A61B 3/04; A61B 3/005; A61B 3/063

(57) ABSTRACT

A patient's contact lens fitting profile can be performed using an electronic device in a virtual environment. The electronic device can execute a visual assessment application and display visual content continuously for an extended duration of time in the 3D virtual environment. The visual content can be displayed with predefined display parameters associated with contact lens fitting. The electronic device can obtain a stream of sensor data measured by the one or more sensors and apply at least a contact lens fitting model to generate a contact lens fitting profile for a user associated with the electronic device based on the stream of sensor data.

### TECHNICAL FIELD

The present disclosure relates to vision test technology. More specifically, methods, systems, devices, and non-statutory computer-readable storage media can be applied to analyze contact lens fitting in an extended reality environment.

### BACKGROUND

Traditional methods for visual acuity assessment do not allow for dynamic adjustment of test parameters, leading to less accurate assessments, nor can they be implemented to test eyes and vision at home using household devices in a very environment locked manner.

### SUMMARY

The present disclosure relates to innovative methods and systems that can revolutionize vision care, making vision testing and other exams more accessible and affordable for patients. Additionally, it is contemplated that the principles and features of the present disclosure can be implemented in numerous other applications of display technology, including headsets, heads-up displays, and other microdisplays (e.g., microLED and microOLED) to address challenges and limitations inherent in such products and their uses.

Some implementations of the present disclosure are directed to a method for testing vision. The method is implemented at an electronic device having a head-mounted display (HMD), one or more processors, and memory. The method includes executing a visual assessment application, including displaying a user interface to create a 3D virtual environment; partitioning a field of view displayed on the user interface into a plurality of regions; for each of the plurality of regions in the field of view, successively: rendering a respective visual pattern in the respective region; obtaining a user response to the respective visual pattern; adjusting a respective vision correction filter to the respective visual pattern based on the user response; and combining respective vision correction filters corresponding to the plurality of regions to determine a prescription of an eyewear for a user associated with the electronic device.

Some implementations of the present disclosure are directed to a method for testing vision. The method is implemented at an electronic device having a head-mounted display (HMD), one or more processors, and memory. The method includes executing a visual assessment application, including displaying a user interface to create a 3D virtual environment; partitioning a field of view displayed on the user interface into a plurality of regions; determining one or more respective corrective measures for each of the plurality of regions in the field of view; and determining a prescription of an eyewear for a user associated with the electronic device, wherein the prescription of the eyewear includes a map associating each of the plurality of regions with one or more respective corrective measures.

Some implementations of the present disclosure are directed to a method for testing vision. The method is implemented at an electronic device having an HMD, one or more processors, and memory. The method includes executing a visual assessment application, including displaying a user interface to create a 3D virtual environment corresponding to a field of view of a user associated with the electronic device; rendering a visual pattern in the field of view; applying a vision correction filter to the visual pattern; obtaining a set of user response data captured by a plurality of sensors in response to the visual pattern; determining whether the set of user response data satisfy a response quality criterion; and dynamically adjusting filter parameters of the vision correction filter based on the set of user response data, until the set of user response data satisfy the response quality criterion.

Some implementations of the present disclosure are directed to a method for testing vision. The method is implemented at an electronic device having an HMD, one or more processors, and memory. The method includes rendering a visual pattern in the field of view; applying a vision correction filter to the visual pattern; obtaining a set of user response data captured by a plurality of sensors in response to the visual pattern; adjusting filter parameters of the vision correction filter based on the set of user response data; and in accordance with a determination that the set of user response data satisfy a response quality criterion, generating a prescription of an eyewear based on the filter parameters of the vision correction filter.

Some implementations of the present disclosure are directed to a method for testing vision. The method is implemented at an electronic device having an HMD, one or more processors, and memory. The method includes executing a visual assessment application, including displaying a user interface to create a 3D virtual environment; displaying visual content continuously for an extended duration of time in the 3D virtual environment, wherein the visual content is displayed with predefined display parameters associated with a screen usage; obtaining a stream of sensor data measured by the one or more sensors; determining a plurality of sequential user responses to the visual content based on the stream of sensor data; and applying at least a screen usage prediction model to generate a screen usage guidance profile for the user based on the plurality of sequential user responses.

Some implementations of the present disclosure are directed to a method for testing vision. The method is implemented at an electronic device having an HMD, one or more processors, and memory. The method includes displaying visual content continuously for an extended duration of time in a 3D virtual environment, wherein the visual content is displayed with predefined display parameters associated with a screen usage; obtaining a stream of sensor data; determining a plurality of sequential user responses to the visual content based on the stream of sensor data; and generating a screen usage guidance profile for the user based on the plurality of sequential user responses, the screen usage guidance profile including at least a time-dependent display parameter.

Some implementations of the present disclosure are directed to a method for testing vision. The method is implemented at an electronic device having an HMD, one or more processors, and memory. The method includes executing a visual assessment application, including displaying a user interface to create a 3D virtual environment; displaying a predefined video clip in the 3D virtual environment, the predefined video clip including a plurality of visual sessions corresponding to a sequence of vision tests; while the predefined video clip is played; obtaining a stream of sensor data measured by the one or more sensors; and determining a plurality of first response parameters to the sequence of vision tests based on the stream of sensor data.

Some implementations of the present disclosure are directed to a method for testing vision. The method is implemented at an electronic device having an HMD, one or more processors, and memory. The method includes displaying a predefined video clip in a 3D virtual environment, the predefined video clip including a plurality of visual sessions corresponding to a sequence of vision tests; while the predefined video clip is played, obtaining a stream of sensor data measured by the one or more sensors; determining a current response feature vector indicating a user response to the sequence of vision tests based on the stream of sensor data; and determining a chronic vision change of a user associated with the electronic device based on a plurality of response feature vectors including a current response feature vector.

Some implementations of the present disclosure are directed to a method for testing vision. The method is implemented at an electronic device having an HMD, one or more processors, and memory. The method includes executing a visual assessment application, including displaying a user interface to create a 3D virtual environment; identifying a plurality of horizontal lines of sight; for each horizontal line of sight: rendering a respective visual stimulus on the respective horizontal line of sight; obtaining a user response to the respective visual stimulus; dynamically adjusting stimulus parameters of the respective visual stimulus based on the user response; and based on the stimulus parameters associated with each horizontal line of sight, determining an eyewear prescription of an eyewear for a user associated with the electronic device, the eyewear prescription including prescription parameters corresponding to the plurality of horizontal lines of sight.

Some implementations of the present disclosure are directed to a method for testing vision. The method is implemented at an electronic device having an HMD, one or more processors, and memory. The method includes identifying a plurality of horizontal lines of sight; rendering a visual stimulus successively on the plurality of horizontal lines of sight; dynamically adjusting stimulus parameters of the visual stimulus based on a spontaneous user response; and based on the stimulus parameters, determining an eyewear prescription of an eyewear for a user associated with the electronic device, the eyewear prescription including corrective measurements corresponding to the plurality of horizontal lines of sight.

Some implementations of the present disclosure are directed to a method for testing vision. The method is implemented at an electronic device having an HMD, one or more processors, and memory. The method includes determining a multifocal eyewear prescription of a user associated with the electronic device, wherein the multifocal eyewear prescription includes a multifocal parameter for a lens having a plurality of focal lengths; partition a field of view displayed on the user interface into a plurality of regions; displaying a visual stimulus successively in two distinct regions of the user interface; obtaining user response data captured by one or more sensors in response to the visual stimulus displayed in the two distinct regions; and based on the user response data, adjusting the multifocal parameter of the multifocal eyewear prescription.

Some implementations of the present disclosure are directed to a method for testing vision. The method is implemented at an electronic device having an HMD, one or more processors, and memory. The method includes obtaining a multifocal eyewear prescription of a user associated with the electronic device, wherein the multifocal eyewear prescription includes a multifocal parameter for a lens having a plurality of focal lengths; based on the multifocal parameter, displaying a visual stimulus successively in a plurality of distinct regions of a 3D virtual environment; obtaining a spontaneous user response in response to the visual stimulus displayed in the two distinct regions; and based on the spontaneous user response, automatically, adjusting the multifocal parameter of the multifocal eyewear prescription.

Some implementations of the present disclosure are directed to a method for testing vision. The method is implemented at an electronic device having an HMD, one or more processors, and memory. The method includes executing a visual assessment application, including displaying a user interface to create a 3D virtual environment; displaying visual content continuously for an extended duration of time in the 3D virtual environment, wherein the visual content is displayed with predefined display parameters associated with contact lens fitting; obtaining a stream of sensor data measured by the one or more sensors; and applying at least a contact lens fitting model to generate a contact lens fitting profile for a user associated with the electronic device based on the stream of sensor data.

Some implementations of the present disclosure are directed to a method for testing vision. The method is implemented at an electronic device having an HMD, one or more processors, and memory. The method includes executing a visual assessment application, including displaying a user interface to create a 3D virtual environment; obtaining a plurality of eye images captured by an eye-tracking camera; and generating a current contact lens fitting profile for a user associated with the electronic device based on the plurality of eye images.

Some implementations of the present disclosure are directed to a method for testing vision. The method is implemented at an electronic device having an HMD, one or more processors, and memory. The method includes executing a visual assessment application, including displaying a user interface to create a 3D virtual environment; obtaining a comprehensive prescription for an eyewear having a plurality of lens portions, each lens portion corresponding to a distinct region of a field of view and having a respective prescription parameter; generating a bifocal filter, a trifocal filter, and/or a progressive filter based on the comprehensive prescription; obtaining 3D visual content for display on the user interface; and rendering a plurality of versions of the first 3D visual content based on the bifocal filter, the trifocal filter, and/or the progressive filter.

Some implementations of the present disclosure are directed to a method for testing vision. The method is implemented at an electronic device having an HMD, one or more processors, and memory. The method includes executing a visual assessment application, including displaying a user interface to create a 3D virtual environment; obtaining a comprehensive prescription for an eyewear having a plurality of lens portions, each lens portion corresponding to one or more respective regions of a field of view and having a respective prescription parameter; obtaining 3D visual content for display on the user interface; and generating a multifocal prescription, including iteratively: rendering the 3D visual content based on the comprehensive prescription; and simplifying the comprehensive prescription, until an eyewear fitting condition is satisfied.

In some embodiments, a user application can be implemented by an electronic device including an HMD and configured to create a customized extended reality (XR) environment for a user engaged on an XR information platform. Products may be rendered for the user in a three-dimension format in the XR environment, thereby facilitating eyewear selection and fitting. The XR can be an umbrella term encapsulating Augmented Reality (AR), Virtual Reality (VR), Mixed Reality (MR), and everything in between. In this application, any embodiments that apply a VR system can be implemented using an AR or MR system as well.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE FIGURES

Various features of illustrative embodiments of the inventions are described below with reference to the drawings. The illustrated embodiments are intended to illustrate, but not to limit, the inventions.

FIG. 1 is an example data processing environment having one or more servers communicatively coupled to one or more computer devices (e.g., a headset device), in accordance with some embodiments.

FIG. 2 is an environment in which a computer device (e.g., a headset device) is applied to facilitate visual assessment or eyewear fitting, in accordance with some embodiments.

FIG. 3 is a block diagram of a computer system (e.g., including a headset device) configured to implement vision assessment or eyewear fitting, in accordance with some embodiments.

FIG. 4 is a block diagram of a machine learning system for training and applying machine learning models (e.g., for glass making), in accordance with some embodiments.

FIG. 5A is a structural diagram of an example neural network applied to process input data in a machine learning model, and FIG. 5B is an example node in the neural network, in accordance with some embodiments.

FIG. 6A is an example tumbling E chart applied in a visual acuity test, and FIGS. 6B-6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIG. 11 is a flow diagram of an example vision test process for determining a detailed eyewear prescription based on a plurality of regions (also called focus areas), in accordance with some embodiments.

FIG. 12 is a flow diagram of an example vision test process for determining an eyewear prescription, in accordance with some embodiments.

FIG. 13 is a schematic diagram of an example field of view including a plurality of regions, in accordance with some embodiments.

FIG. 14 is a flow diagram of an example vision test process for determining corrective measures based on vision correction simulation, in accordance with some embodiments.

FIG. 15 is a flow diagram of an example vision test process for simulating vision correction, in accordance with some embodiments.

FIG. 16 is a flow diagram of an example response processing method for determining response parameters, in accordance with some embodiments.

FIG. 17 is a flow diagram of an example vision test process for evaluating visual susceptibility of a user's eyes to digital screen exposure, in accordance with some embodiments.

FIG. 18 is a flow diagram of an example vision test process for assessing digital screen use by a user 120, in accordance with some embodiments.

FIG. 19 is a flow diagram of an example vision test process for evaluating prescription changes in a 3D virtual environment, in accordance with some embodiments.

FIG. 20 is a flow diagram of an example vision test process for tracking a chronic eye condition, in accordance with some embodiments.

FIG. 21A is a flow diagram of an example response processing method for determining response parameters, in accordance with some embodiments.

FIG. 21B is a flow diagram of an example response processing method for determining a chronic vision change, in accordance with some embodiments.

FIG. 22 is a flow diagram of an example vision test process for determining optimal vision correction parameters through successive approximation, in accordance with some embodiments.

FIG. 23A is a diagram of an example horizontal field of view (HFOV) of a user's eyes, in accordance with some embodiments, and FIG. 23B is a schematic diagram of an example field of view including three rows of regions corresponding to a plurality of lines of sight, in accordance with some embodiments.

FIG. 24 is a flow diagram of an example vision test process for determining an eyewear prescription corresponding to a plurality of horizontal lines of sight, in accordance with some embodiments.

FIG. 25 is a flow diagram of an example vision test process for prescribing and adjusting multifocal lenses in a 3D virtual environment, in accordance with some embodiments.

FIG. 26 is a set of example lenses including one or more focal lengths, in accordance with some embodiments.

FIG. 27 is a flow diagram of an example vision test process for determining a multifocal eyewear prescription, in accordance with some embodiments.

FIG. 28 is a flow diagram of an example process for determining a multifocal parameter based on user response data, in accordance with some embodiments.

FIG. 29 is a flow diagram of an example vision test process for contact lens fitting in a 3D virtual environment, in accordance with some embodiments.

FIG. 30 is a flow diagram of an example vision test process for checking contact lens fitting, in accordance with some embodiments.

FIG. 31 is a flow diagram of an example process for rendering content for multifocal eyewear fitting in a 3D virtual environment, in accordance with some embodiments.

FIG. 32 is a flow diagram of an example vision test process for preparing an eyewear, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

Moreover, various aspects of the present disclosure can be implemented in combination with aspects of other virtual-reality technology developed by the present applicant, for example, in copending U.S. Patent App. Nos. 63/560,623 (137034-5002), filed on Mar. 1, 2024, 63/569,095 (137034-5005), filed on Mar. 23, 2024, 63/642,571 (137034-5007), filed on May 3, 2024, 63/642,583 (137034-5009), filed on May 3, 2024, 63/642,593 (137034-5010), filed on May 3, 2024, 63/642,604 (137034-5011), filed on May 3, 2024, 63/644,457 (137034-5012), filed on May 8, 2024, Ser. No. 18/759,641 (137034-5018), filed on Jun. 28, 2024, Ser. No. 18/791,203 (137034-5036), filed on Jul. 31, 2024, Ser. No. 18/827,546, filed Sep. 6, 2024, and Ser. No. 18/827,588, filed Sep. 6, 2024, the entireties of each of which is incorporated herein by reference. Aspects of these copending cases can be implemented in combination with some embodiments disclosed herein, whether in addition to features thereof or as an alternative to a particular feature of an embodiment disclosed herein.

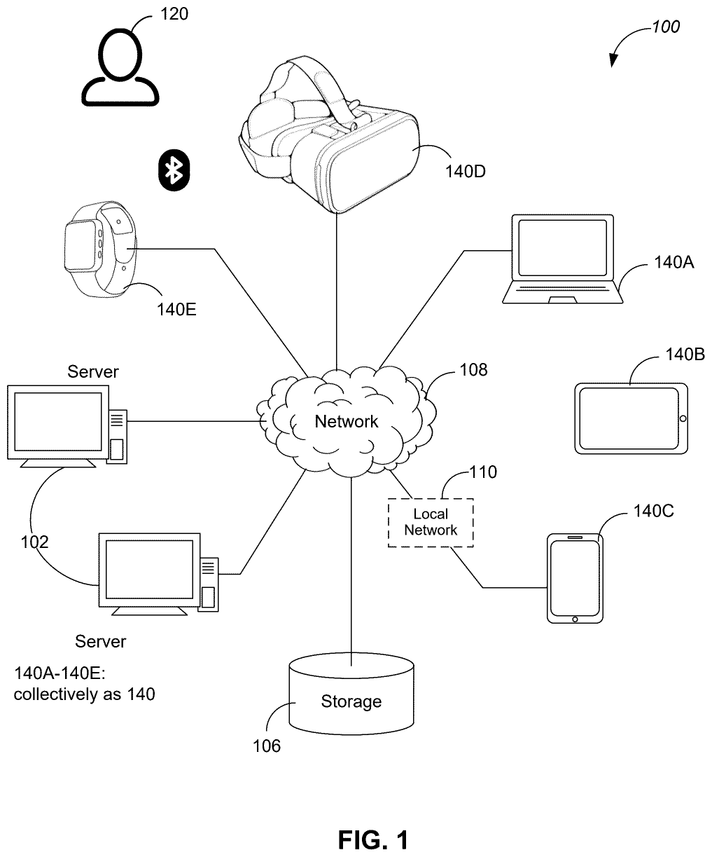

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real time data communication with the computer devices 140 that can be remote from each other or from the one or more servers 102. Further, in some embodiments, the one or more servers 102 can implement data processing tasks that are not completed locally by the computer devices 140. For example, the computer devices 140 can include a game console (e.g., the headset device 140D) that executes an interactive online gaming application (e.g., for visual assessment or eyewear fitting). The game console receives a user instruction and sends it to a server 102 with user data. The server 102 generates a stream of video data based on the user instruction and user data and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound includes user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern. For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

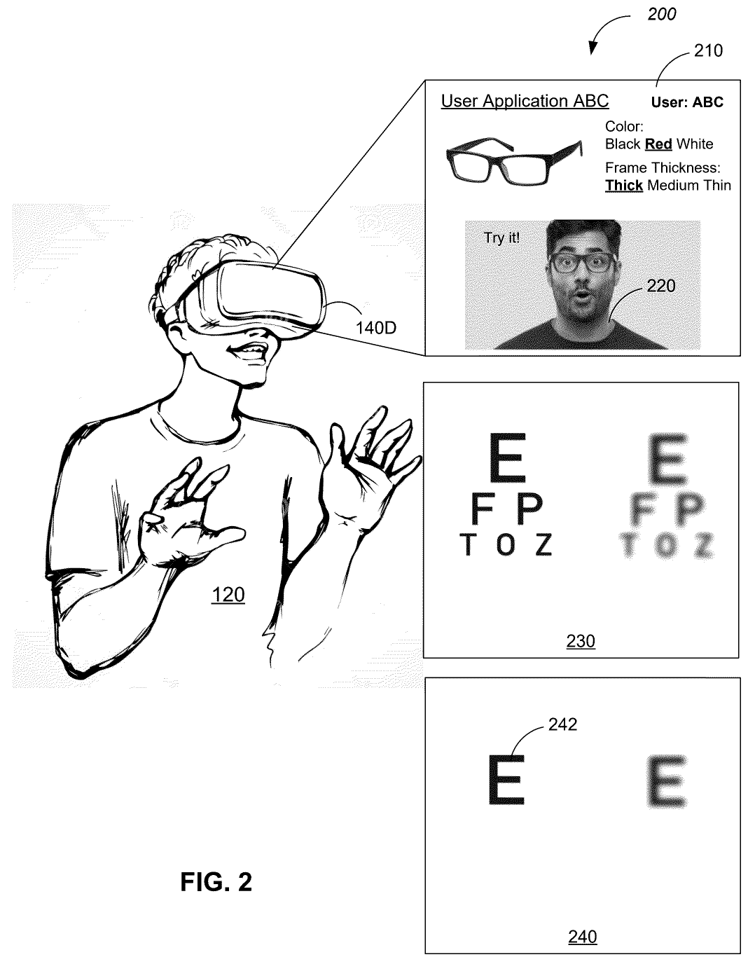

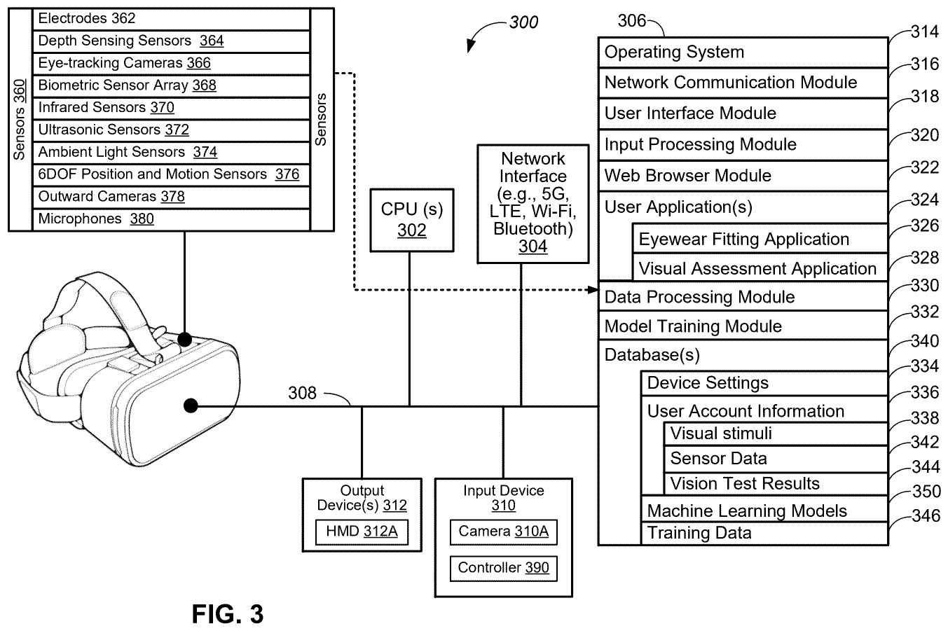

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive, from the first user interface 210, a user feedback message indicating an issue, requesting further improvement, or confirming a fit. In some embodiments, a second user interface 230 can be displayed on a computer device 140 associated with the user 120. The second user interface 230 may include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 may include one or more input devices 310 that facilitate user input, such as a keyboard, a mouse, a voice-command input unit or microphone, a touch screen display, a touch-sensitive input pad, a gesture capturing camera, a controller 390, or other input buttons or controls. Furthermore, in some embodiments, the computer device 140 of the computer system 300 may use a microphone for voice recognition or an eye tracking camera 366 for tracking eyeball movement. In some implementations, the computer device 140 may include one or more optical cameras (e.g., an RGB camera), scanners, or photo sensor units for capturing images. The computer system 300 may also include one or more output devices 312 that enable presentation of user interfaces 210 and media content. The one or more output devices 312 may include one or more speakers and/or one or more visual displays.

The computer system 300 may include one or more sensors 360, which further may include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more microphones 380. It is noted that the one or more sensors 360 can also be included in the input device 310 and used to collect data to the computer system 300.

Memory 306 may include high-speed random-access memory, such as DRAM, SRAM, DDR RAM, or other random-access solid state memory devices; and, optionally, may include non-volatile memory, such as one or more magnetic disk storage devices, one or more optical disk storage devices, one or more flash memory devices, or one or more other non-volatile solid state storage devices. Memory 306, optionally, may include one or more storage devices remotely located from one or more processing units 302. Memory 306, or alternatively the non-volatile memory within memory 306, may include a non-transitory computer readable storage medium. In some implementations, memory 306, or the non-transitory computer readable storage medium of memory 306, may store the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof may include a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training module 332 for obtaining training data 346 and training machine learning models 350; and
One or more databases 340 for storing at least data including one or more of:
   Device settings 334 including common device settings (e.g., service tier, device model, storage capacity, processing capabilities, communication capabilities, etc.) of the computer system 300;
   User account information 336 for the one or more user applications 324, e.g., user names, security questions, account history data, user preferences, and predefined account settings, where in some embodiments, the user account information 336 may include facial measurements and one or more virtual fitting parameters associated with associated with a user account of an eye fitting application 326, and in some embodiments, the user account information 336 may include visual stimuli 338, sensor data 342, and vision test results 344 associated with a user account of a visual assessment application 328; and
   Machine learning models 350 including parameters (e.g., weights, biases) used to implement vision test or select eyewear for eyewear users.

Each of the above identified elements may be stored in one or more of the previously mentioned memory devices and correspond to a set of instructions for performing a function described above. The above identified modules or programs (i.e., sets of instructions) need not be implemented as separate software programs, procedures, modules or data structures, and thus various subsets of these modules may be combined or otherwise rearranged in some embodiments. In some embodiments, memory 306, optionally, stores a subset of the modules and data structures identified above. Furthermore, memory 306, optionally, stores additional modules and data structures not described above.

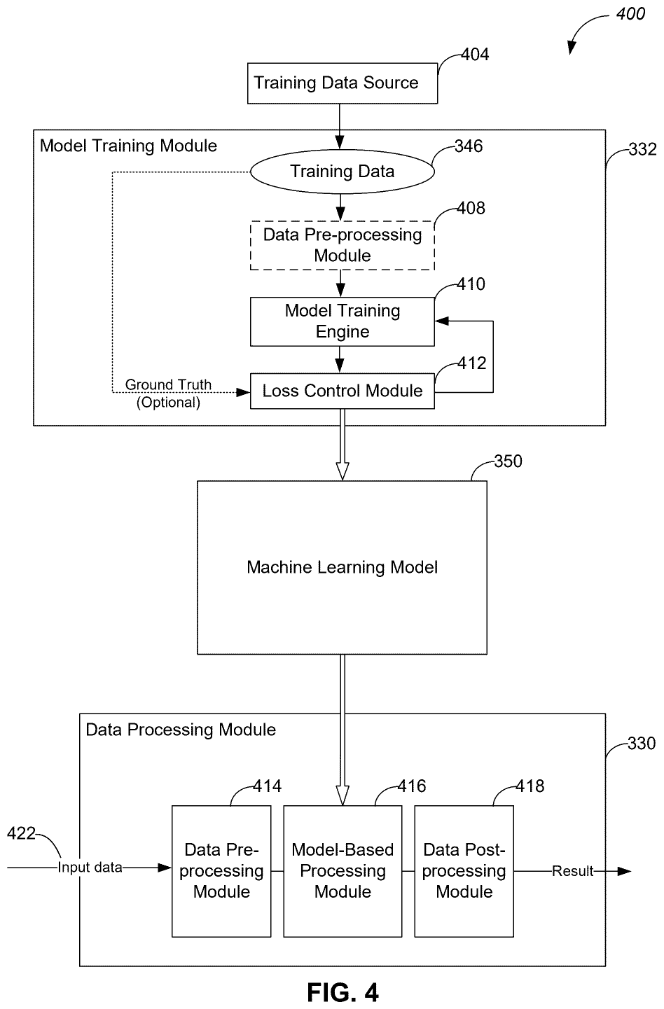

FIG. 4 is a block diagram of a machine learning system 400 for training and applying machine learning models 350 (e.g., for glass making), in accordance with some embodiments. The machine learning system 400 may include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 may include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real time input data 422 detected by the computer device 140. In some embodiments, the training data 346 provided by the training data source 404 may include a standard dataset widely used to train machine learning models 350. The input data 422 further may include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 may include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criteria (e.g., a comparison result of the loss function is minimized or reduced below a loss threshold). The machine learning models 350 may thereby be trained and provided to the data processing module 330 of a computer device 140 to process real time input data 422 from the computer device 140.

In some embodiments, the model training module 402 may further include a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and include a desired output for each training data item (also called the ground truth, in some embodiments). In some embodiments, the desirable output may be labelled manually by people or automatically by the model training module 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 may include a data pre-processing module 414, a model-based processing module 416, and a data post-processing module 418. The data pre-processing modules 414 may pre-process input data 422 based on the type of the input data 422. In some embodiments, functions of the data pre-processing modules 414 are consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

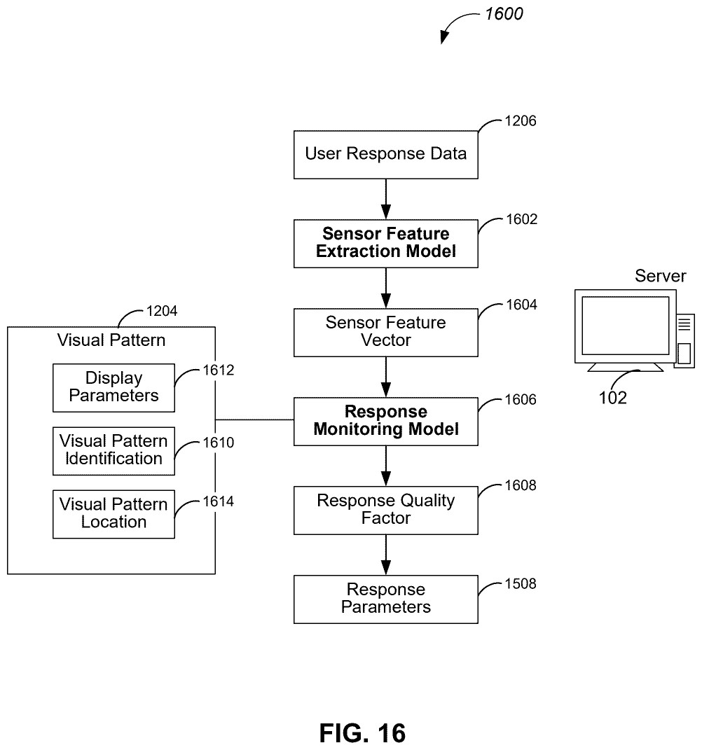

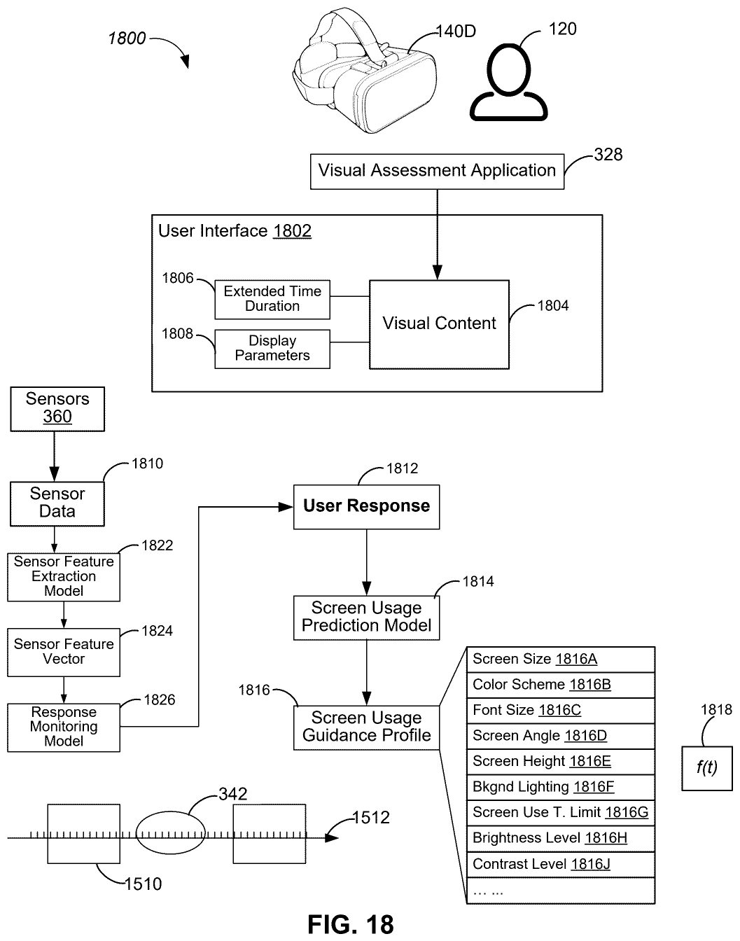

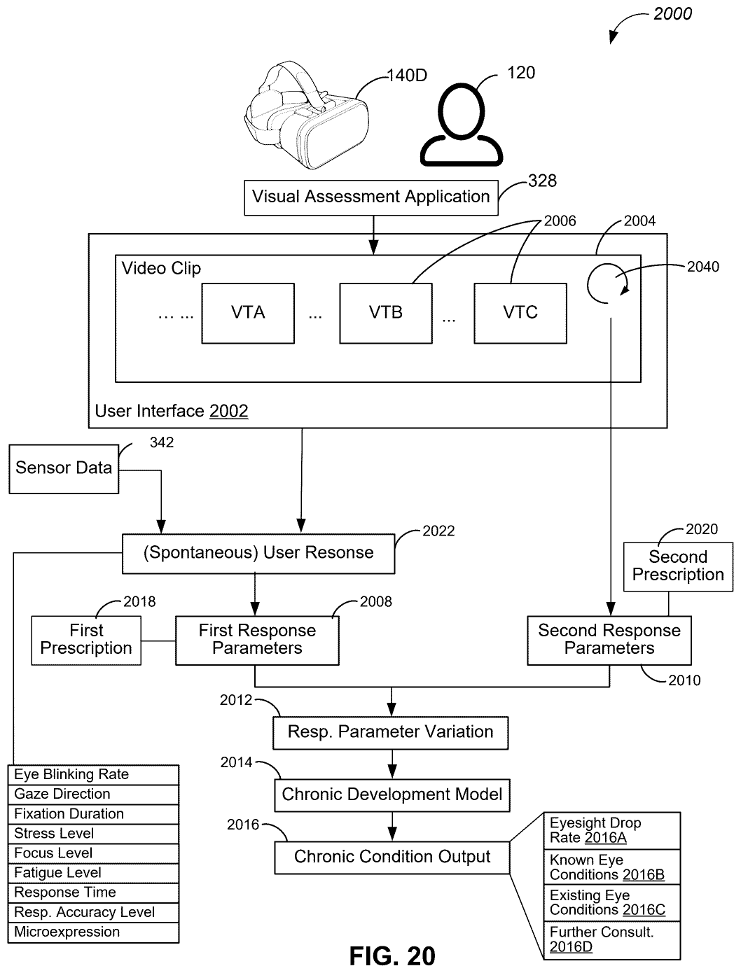

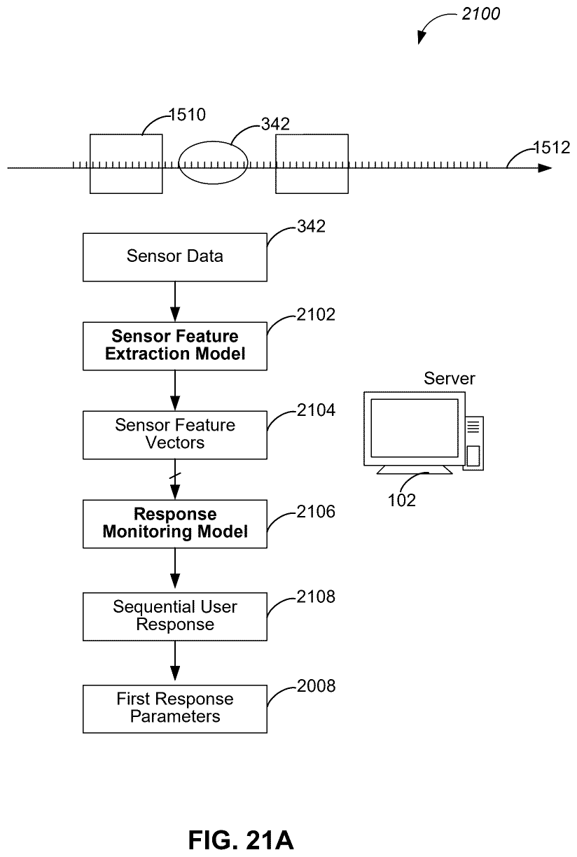

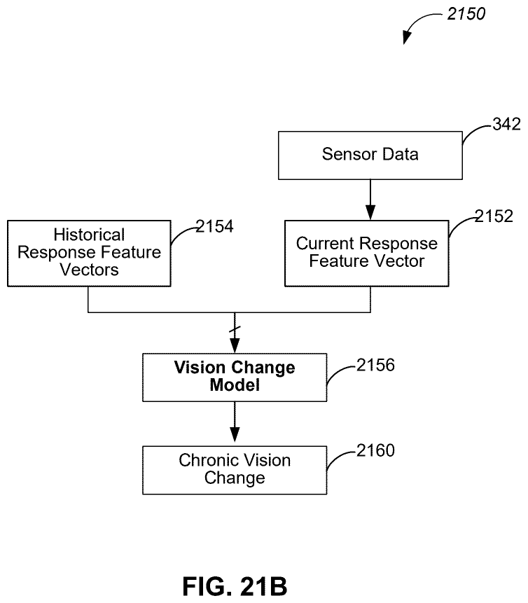

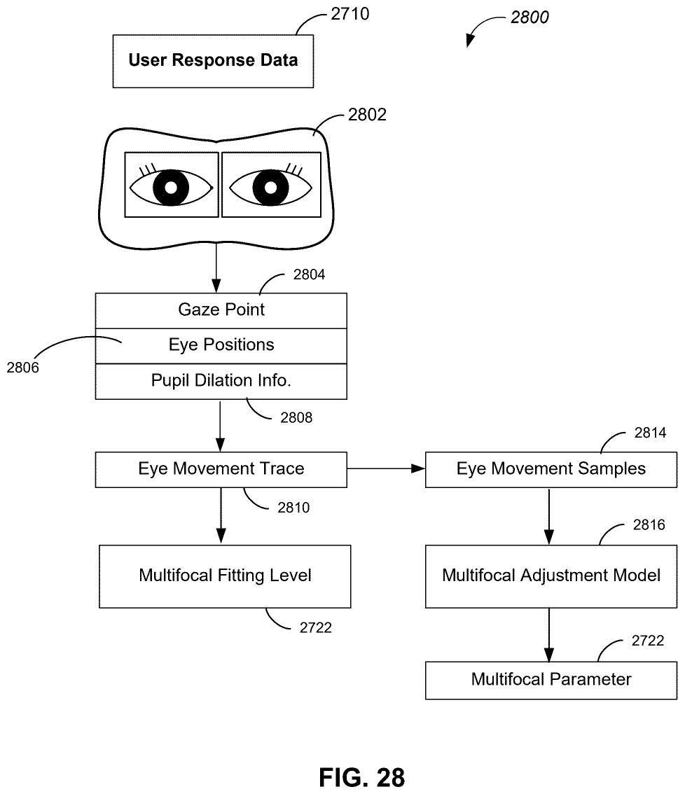

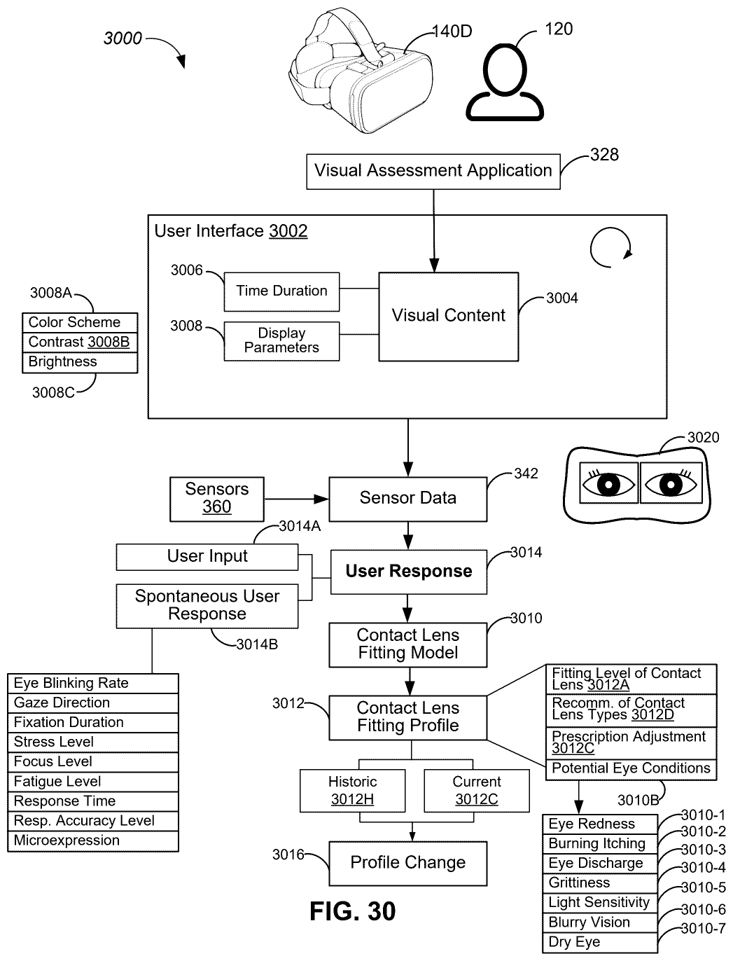

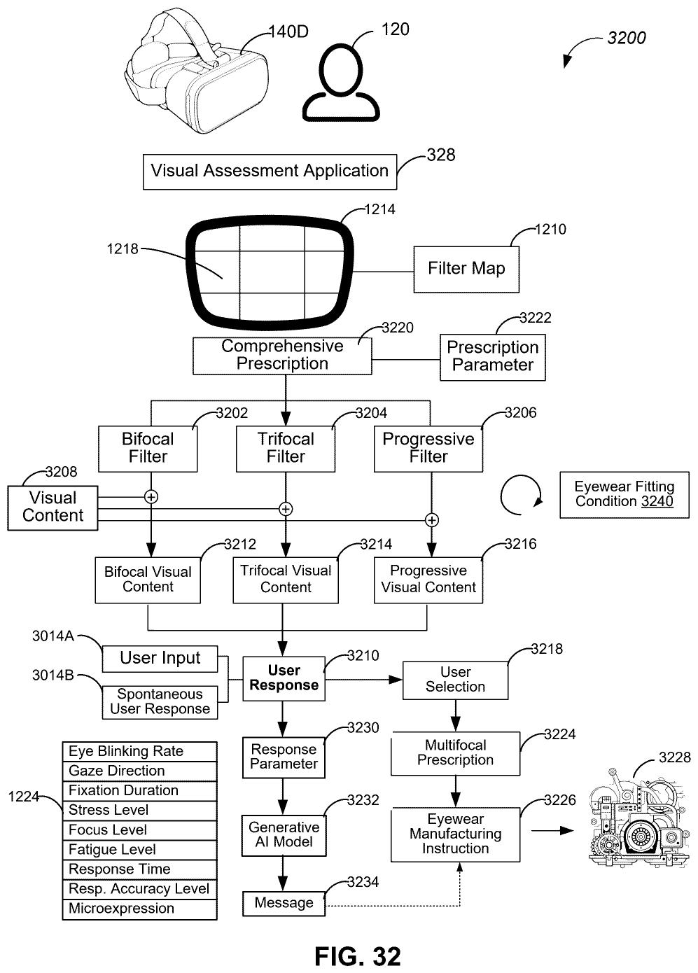

Examples of the machine learning model 350 include, but are not limited to, sensor feature extraction models (FIGS. 16, 18, 21), response monitoring models (FIG. 16, screen usage prediction model 1814 (FIG. 18), chronic development model 2014 (FIG. 20), vision change model 2156 (FIG. 21), multifocal adjustment model 2816 (FIG. 28), contact lens fitting model 3010 (FIG. 30), and generative artificial intelligence (AI) model 3232 (FIG. 32).

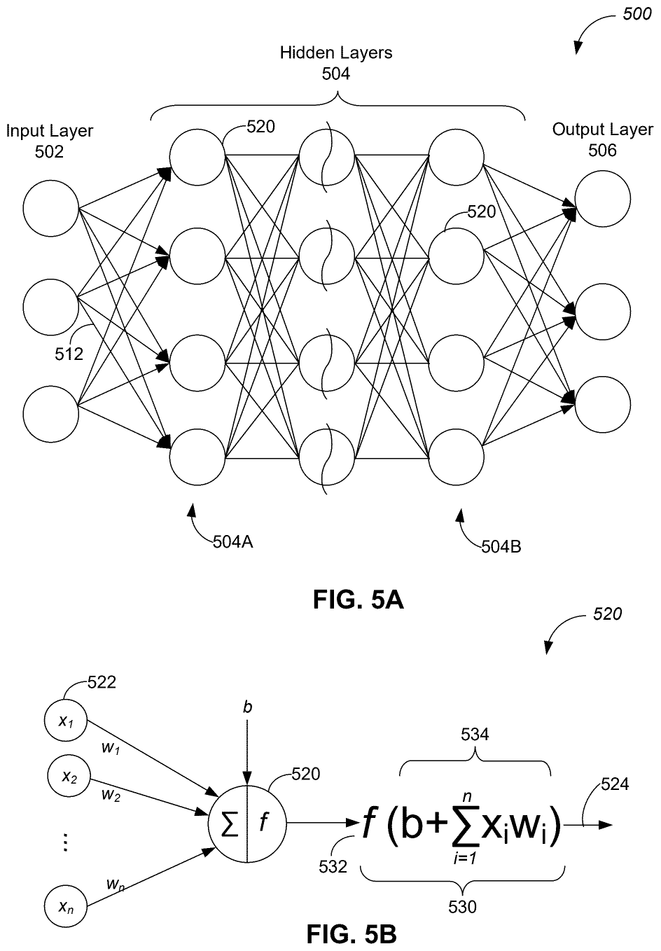

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 may include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers may include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a fully connected layer because each node in the layer is connected to every node in its immediately following layer. In some embodiments, a hidden layer 504 may include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN include convolutional layers. Each node in a convolutional layer may receive inputs from a receptive area associated with a previous layer (e.g., nine nodes). Each convolution layer may use a kernel to combine pixels in a respective area to generate outputs. For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights wᵢ for each layer of the neural network 500 using training data 346 that is provided in the input layer 502. The training process typically may include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training may include a network bias parameter b for each layer.

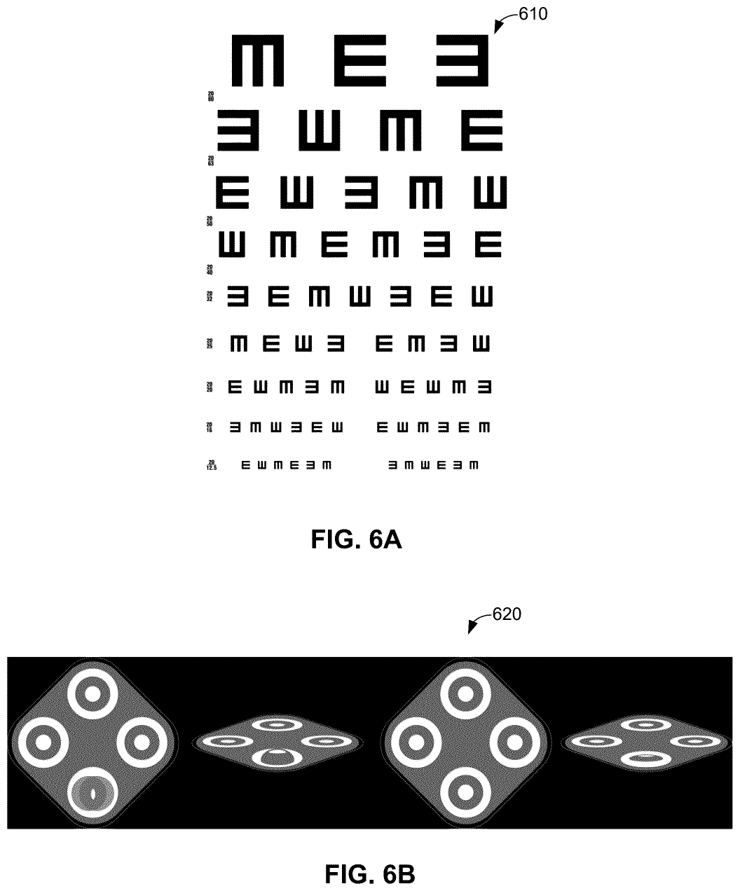

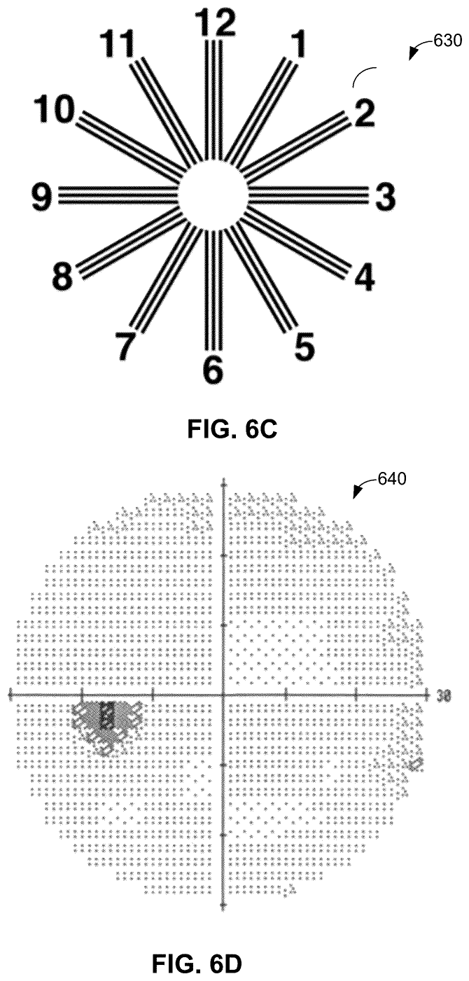

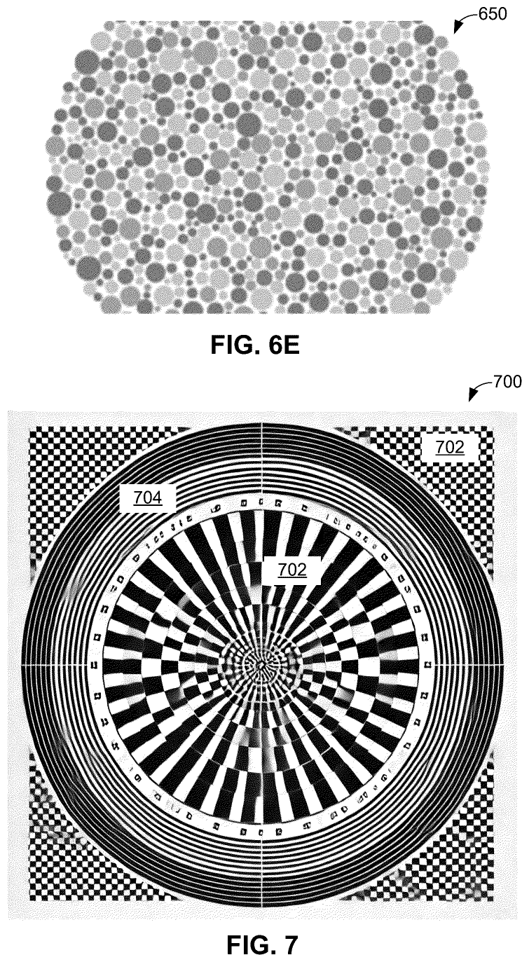

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example tumbling E chart 610 applied in a visual acuity test, in accordance with some embodiments. FIGS. 6B, 6C, 6D, and 6E are example patterns 620, 630, 640, and 650 applied in a stereopsis test, an astigmatism test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

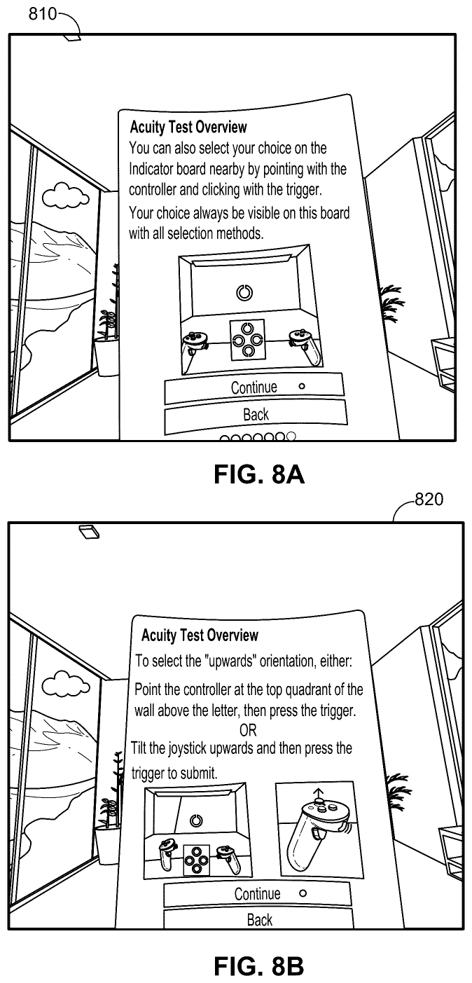

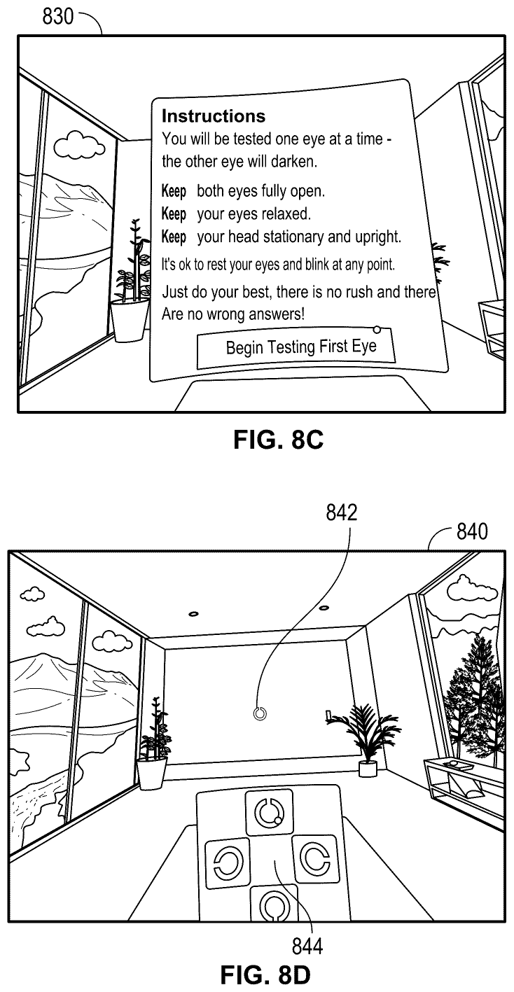

FIGS. 8A-8D include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using a controller 390 (FIG. 3) to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

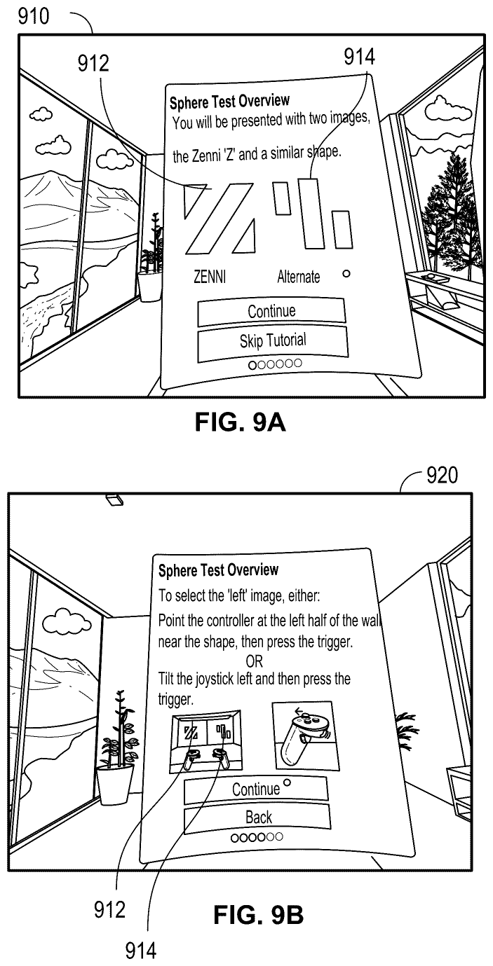

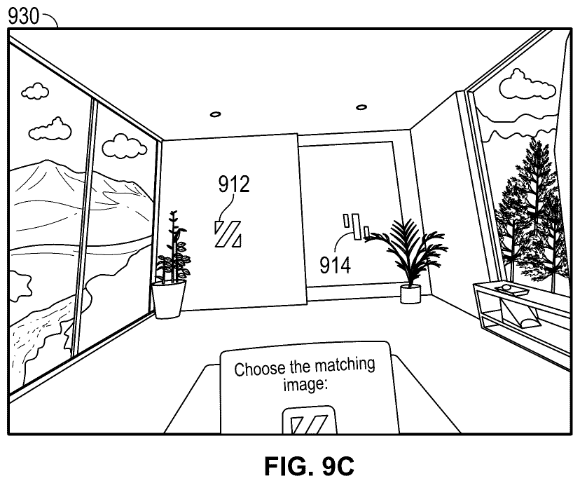

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using a controller 390 (FIG. 3) to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

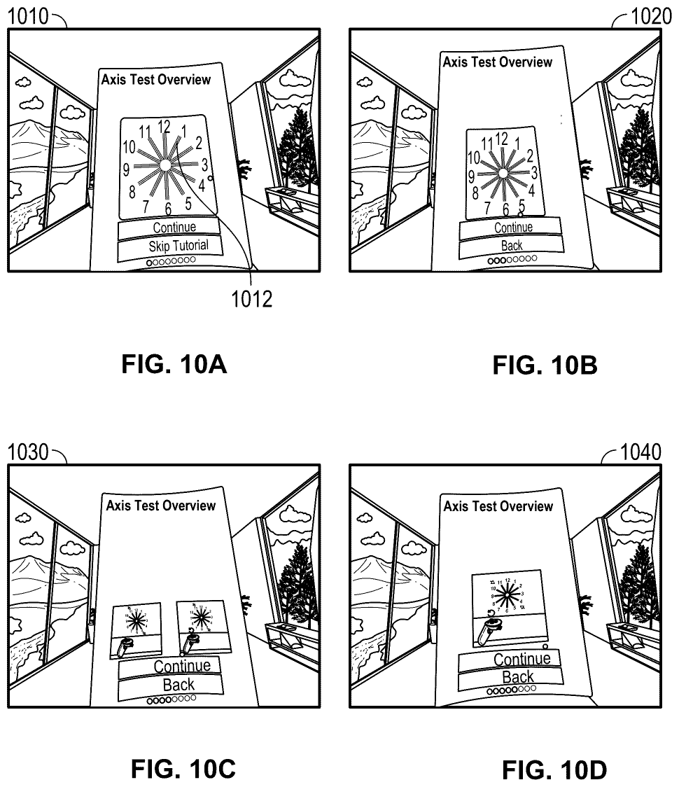

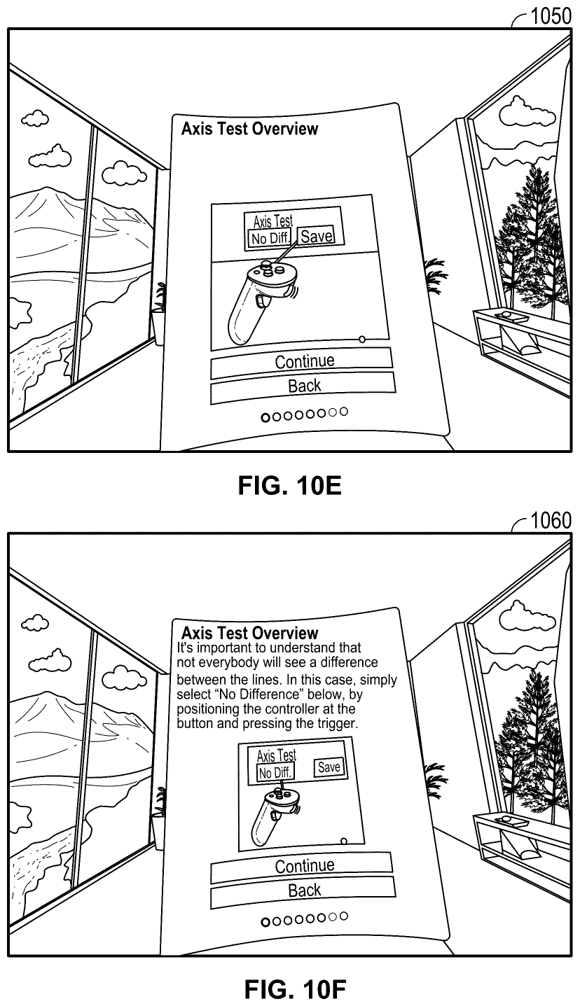

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 may include a message, e.g., You will be presented with a clock diagram of converging numbered lines. The user interface 1020 may display an information page explaining what is selected on the clock diagram of converging numbered lines 1012 displayed in the virtual environment. For example, the user interface 1010 may include a message, e.g., Your task is to identify if any of these sets of lines appear clearer, crisper, or darker than other. The user interface 1030 may display an information page including two optional ways of using a controller 390 (FIG. 3) to select lines on the clock diagram of converging numbered lines 1012. For example, the user interface 1010 may include a message, e.g., Make a selection by either pointing the controller 390 at the lines on the clock, then pressing the trigger and rotating the joystick to move the indicator arrows around the clock. The user interface 1040 may display an information page illustrating an embodiment having equally clear lines on the clock diagram of converging numbered lines 1012. For example, the user interface 1010 may include a message, e.g., If two sets of neighboring lines seem to both stand out as equally clear, you can move the indicator arrows to a halfway point between those lines.

Referring to FIG. 10E, the user interface 1050 may display an information page including an instruction using the controller 390 to submit a selection. For example, the user interface 1010 may include a message, e.g., After selecting a set of lines, submit your choice with the ‘Done’ button below by pointing to the controller 390 at the button and pressing the trigger. Further, referring to FIG. 10F, the user interface 1060 may display an information page including an instruction using the controller 390 to indicate that no difference is observed on the clock diagram of converging numbered lines 1012. For example, the user interface 1010 may include a message, e.g., It's important to understand that not everybody will see a difference between the lines and in this case, simply select ‘No Difference’ below, by positioning the controller at the button and pressing the trigger.

### Multi-Region Eyewear Prescription

Some implementations of this application include a VR-based computer system 300 configured to determine optimal eyeglass prescriptions through the use of dynamic focus areas. The computer system 300 may utilize a high-resolution VR headset that may be equipped with eye-tracking sensors (e.g., eye-tracking cameras 366 in FIG. 3) and a visual assessment application 328 to generate an interactive visual environment with adjustable focus zones. Users may wear the VR headset and participate in a series of tasks that require a user to shift a focus between different areas and objects. The eye-tracking sensors may continuously monitor the user's gaze direction, fixation duration, and focus adjustments, while the visual assessment application 328 dynamically alters the focus areas to simulate lens prescriptions, thereby enabling real-time assessment of visual clarity and comfort under different optical conditions.

In some embodiments, the VR-based computer system 300 may incorporate a variety of tasks configured to evaluate the user's visual acuity and focusing ability, e.g., reading text at varying distances, identifying details in complex scenes, and following moving objects. A user application 324 (e.g., visual assessment application 328 in FIG. 3) may process the data and evaluate parameters, such as clarity, sharpness, and user comfort across different simulated prescriptions. The computer system 300 may determine a lens prescription customized based on the user's specific visual needs. Results may be compiled into a report that provides detailed insights into the user's visual performance and the recommended eyeglass prescription. As such, the computer system 300 may offer a dynamic, engaging, and precise approach to prescribing eyeglasses.

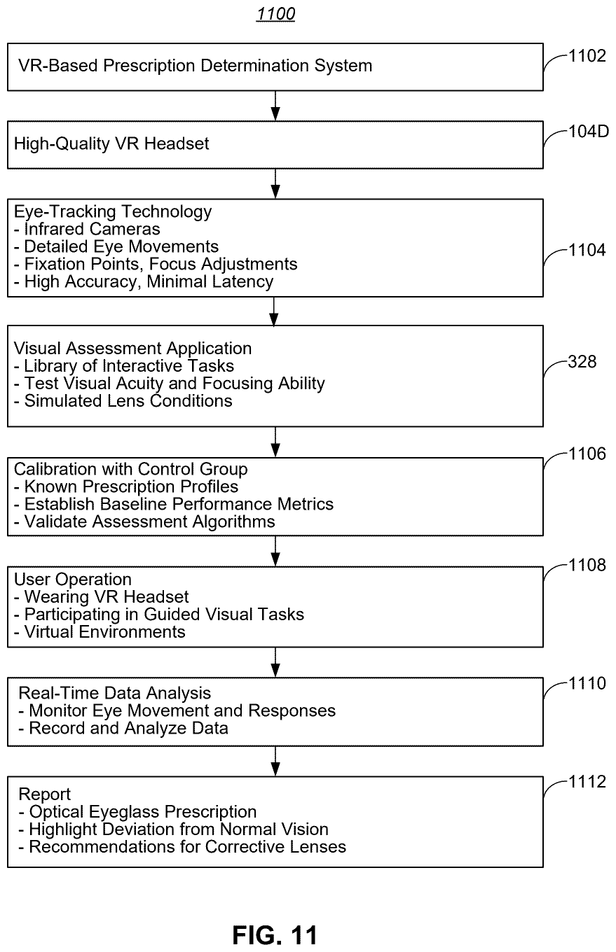

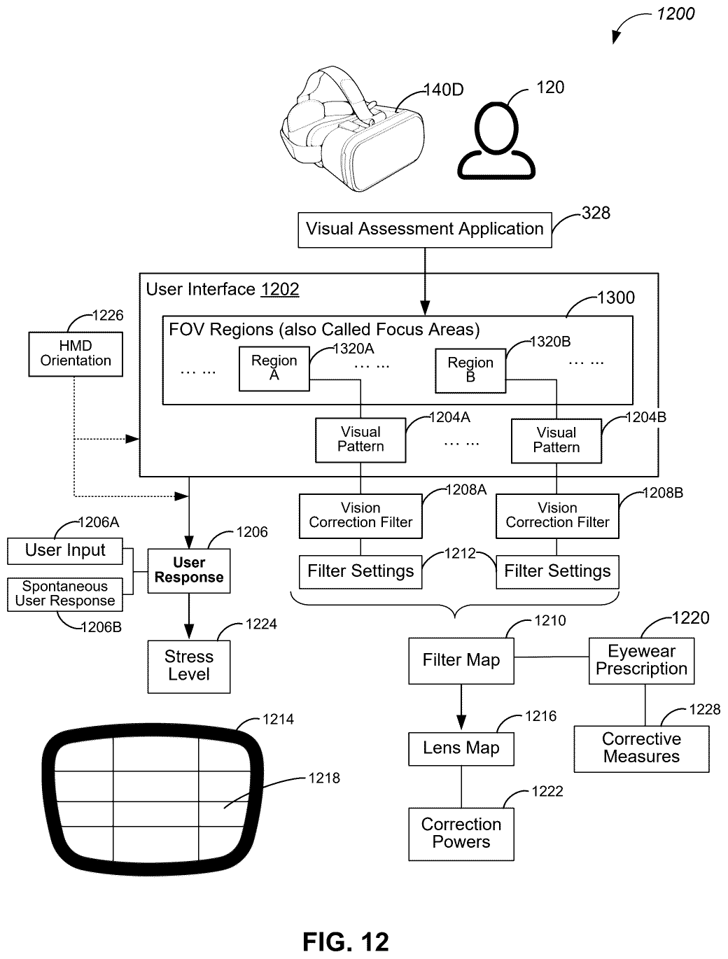

FIG. 11 is a flow diagram of an example vision test process 1100 for determining a detailed eyewear prescription (e.g., prescription 1220 in FIG. 12) based on a plurality of regions (also called focus areas), in accordance with some embodiments. The VR-based computer system 300 may be configured to enable a VR-based prescription determination system 1102. The computer system 300 may include a VR headset 104D that includes an eye-tracking camera 366 (FIG. 3). The eye-tracking technology may include an infrared camera (e.g., camera 366) configured to capture (operation 1104) eye movements, fixation points, and focus adjustments with high accuracy and minimal latency. In some embodiments, when a visual assessment application 328 is executed, a library of interactive cognitive tasks may be applied to test visual acuity and focusing ability under various simulated lens conditions. These tasks may include scenarios where the user may be prompted to read text at different distances, identify fine details in images, and track moving objects within the virtual environment.

In some embodiments, when hardware components and software modules are integrated to form the VR-based prescription determination system 1102, the VR-based computer system 300 may be calibrated (operation 1106) using a control group of individuals with known prescription profiles to establish baseline performance metrics and validate the accuracy of the assessment algorithms. Users can then operate (operation 1108) the system 1102 by wearing the VR headset and participating in the guided visual tasks within the virtual environments. The eye-tracking sensors may monitor their eye movements and responses to the dynamically adjusted focus areas, while the visual assessment application 328 records and analyzes (operation 1110) the data in real time. Based on the user's performance, the system generates a detailed report 1112 outlining the optimal eyeglass prescription, highlighting any deviations from normal vision, and providing recommendations for corrective lenses. By these means, the computer system 300 may offer a precise, non-invasive, and user-friendly method for determining eyeglass prescriptions, providing substantial benefits for both clinical applications and personal eye care routines.

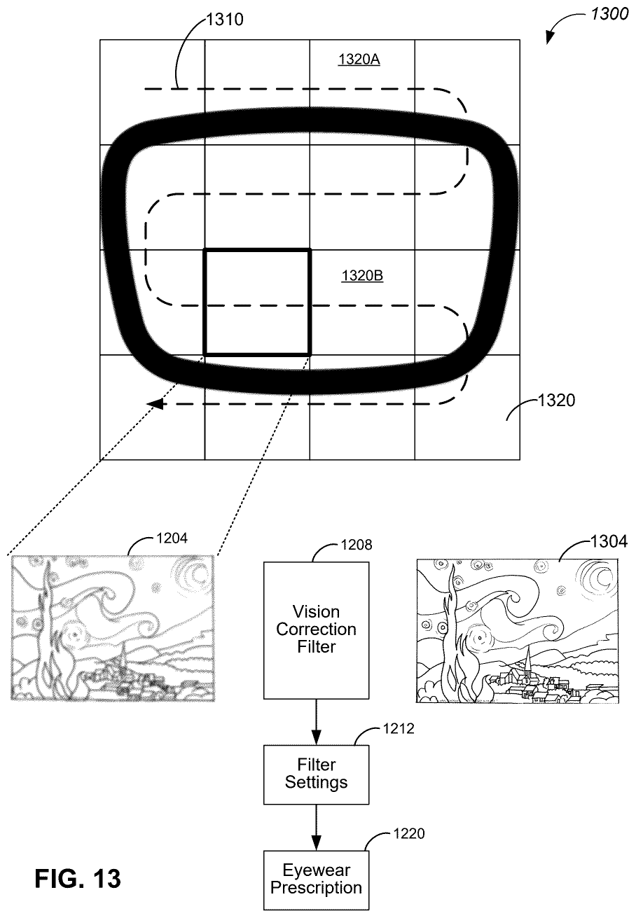

FIG. 12 is a flow diagram of an example vision test process 1200 for determining an eyewear prescription 1220, in accordance with some embodiments, and FIG. 13 is a schematic diagram of an example field of view 1300 including a plurality of regions 1320, in accordance with some embodiments. The eyewear prescription 1220 may correspond to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. In some embodiments, a user 120 may face a field of view 1300 in the 3D virtual environment. Referring to FIG. 13, the field of view 1300 displayed on the user interface 1202 may be partitioned into a plurality of regions 1320. For each of the plurality of regions 1320 in the field of view 1300, the computer device 140 may successively render a respective visual pattern 1204 in the respective region 1320, obtain a user response 1206 to the respective visual pattern 1204, and adjust a respective vision correction filter 1208 to the respective visual pattern 1204 based on the user response 1206. The computer device 140 may combine the respective vision correction filters 1208 corresponding to the plurality of regions 1320 to determine the prescription 1220 of an eyewear for a user 120 associated with the computer device 140. For example, referring to FIG. 12, the computer device 140 may determine the respective vision correction filters 1208A and 1208B for the respective visual patterns 1204A and 1204B displayed on regions 1320A and 1320B, respectively.

In some embodiments, the prescription 1220 of the eyewear may include a filter map 1210 associating the plurality of regions 1320 with the respective vision correction filters 1208. Each region 1320 may be associated with one or more filter settings 1212 of the respective vision correction filter 1208. For example, a region 1320A (e.g., the third region from the left in the top row in FIG. 13) is associated with the filter settings 1212A of the respective vision correction filter 1208A, and a region 1320B (e.g., the third region from the left in the third row in FIG. 13) is associated with the filter settings 1212B of the respective vision correction filter 1208B. Further, in some embodiments, the computer device 140 may identify a selection of an eyewear lens 1214. Based on the selection of the eyewear lens 1214, the computer device 140 may convert the filter map 1210 to a lens map 1216, which associates a plurality of lens portions 1218 of the eyewear lens 1214 with a plurality of correction powers 1222. In some embodiments, the eyewear lens 1214 are not evenly divided to provide the plurality of lens portions 1218. In some embodiments, for each of the plurality of regions 1320, the computer device 140 may identify a respective lens portion 1218 of the eyewear lens 1214, and determine a respective correction power 1222 for the respective lens portion 1218 based on the respective filter settings 1212 of the respective vision correction filter 1208 corresponding to the respective region 1320. In contrast with an existing prescription (having one of a subset of Sphere, Cylinder, Axis, ADD, PD, Prism, and Base), the eyewear prescription 1220 has a higher spatial resolution by including different sets of corrective measures corresponding to different lens portions 1218 of a lens 1214.

In some embodiments, a lens portion 1218 may correspond to two or more regions 1320 of the field of view 1300. The correction power 1222 of the lens portion 1218 may be determined based on the filter settings 1212 of the vision correction filters 1208.

Referring to FIG. 13, in some embodiments, the plurality of regions 1320 may include a first number of regions, and the first number is greater than nine. For example, the field of view 1300 may include sixteen, a hundred, or more than a hundred regions 1320. In some embodiments, the field of view 1300 may be divided substantially evenly to form the plurality of regions 1320. Conversely, in some embodiments, the field of view 1300 may be divided unevenly to form the plurality of regions 1320 (e.g., having a higher density of regions 1320 in a central portion of the field of view 1300 than a peripheral portion of the field of view 1300).

In some embodiments, the eyewear lens 1214 may vary in size and shape, and the plurality of lens portions 1218 may be adaptively determined for the selection of the eyewear lens 1214. In an example, a size of the selected eyewear lens 1214 may allow the plurality of lens portions 1218 to cover a set of central regions (e.g., region 1320B) in their entireties and part of each of a remainder of the regions 1320 (e.g., region 1320A). In an example, each lens portion 1218 may correspond to only part or all of a single region 1320. In another example, a lens portion 1218 may correspond to two or more regions 1320. In some embodiments, the lens portions 1218 match the corresponding regions 1320 of the field of view 1300. Alternatively, in some embodiments, the lens portions 1218 are formed, independently of the regions 1320 of the field of view 1300.

In some embodiments, the plurality of regions 1320 corresponding to a plurality of distinct visual patterns 1204 may jointly form an image frame (e.g., visual pattern 700 in FIG. 7), and the plurality of visual patterns 1204 may be rendered on the user interface 1202 concurrently. The respective vision correction filters 1208 corresponding to the plurality of regions 1320 are adjusted jointly to determine the prescription 1220 of the eyewear. Alternatively, in some embodiments, respective visual patterns 1204 of the plurality of regions 1320 may be rendered successively to determine a respective subset of the prescription 1220 for each respective region 1320. The respective visual patterns 1204 rendered in the plurality of regions 1320 may be the same visual pattern or different visual patterns. In some situations, the respective visual patterns 1204 may be rendered in the plurality of regions 1320 according to a predefined order 1310 associated with positions of the visual patterns 1204. For example, in accordance with the predefined order 1310, the respective visual patterns 1204 may be rendered successively from the top row to the bottom row and from the left to the right within each individual row. Conversely, in some situations, the respective visual patterns 1204 may be rendered in the plurality of regions 1320 according to a random order.

Referring to FIG. 12, in some embodiments, for each of the plurality of regions 1320, the computer device 140 may determine a stress level 1224 based on the user response 1206 to the respective visual pattern 1204 displayed in the respective region 1320. In accordance with a determination that the stress level 1224 satisfies a response criterion, the respective region 1320 may be associated with the respective vision correction filter 1208. The stress level 1224 may be a spontaneous user response 1206B monitored by a subset of one or more second sensors of the computer device 140. In some embodiments, the user response 1206 may include the spontaneous user response 1206B, which are determined based on sensor data 372 (FIG. 3) captured by one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362.

In some embodiments, the user response 1206 may include a user input 1206A captured by a subset of one or more first sensors of the computer device 140, and the one or more first sensors include a forward facing camera 378 (FIG. 3) for detecting a hand gesture, a microphone 380 (FIG. 3) for collecting an audio response, or a controller 390 (FIG. 3) for receiving a user physical force. For example, referring to FIG. 13, the filter settings 1212 of the vision correction filter 1208 may be dynamically adjusted for each of a subset of regions 1320, until the hand gesture, audio response, or user physical force indicates that the visual pattern 1204 has been observed with a satisfactory level of image quality (e.g., in a resulting visual pattern 1304).

In some embodiments, the computer device 140 may determine whether the HMD 312A is oriented forward (e.g., has a predefined HMD orientation 1226), and the visual pattern 1204 may be rendered and the user response 1206 is obtained and processed in accordance with a determination that the HMD 312A is oriented forward.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. The computer device 140 may partition the field of view 1300 displayed on the user interface 1202 into a plurality of regions 1320 and may determine one or more respective corrective measures 1228 for each of the plurality of regions 1320 in the field of view 1300. The prescription 1220 of an eyewear is determined for a user 120 associated with the computer device 140. The prescription 1220 of the eyewear may include a map associating each of the plurality of regions 1320 with the one or more respective corrective measures 1228.

Further, in some embodiments, for each of the plurality of regions 1320 in the field of view 1300, successively, the computer device may render a respective visual pattern 1204 in the respective region 1320, obtain a user response 1206 to the respective visual pattern 1204, adjusting a respective vision correction filter 1208 to the respective visual pattern 1204 based on the user response 1206, and generate the one or more respective corrective measures 1228 based on one or more filter settings 1212 of the respective vision correction filter 1208. Compared with existing eyewear, the process 1200 may provide an eyewear prescription having a high level of granularity of corrective measures.

### Determination of Corrective Measures Based on Vision Correction Simulation

Some implementations of this application include a VR-based computer system 300 configured to prescribe corrective measures for vision through a series of interactive vision correction simulations. The computer system 300 may utilize a high-resolution VR headset that may be equipped with eye-tracking sensors (e.g., eye-tracking cameras 366 in FIG. 3) and a visual assessment application 328 to generate a variety of visual environments that simulate different vision correction scenarios. A user 120 may wear the VR headset and perform a series of tasks that require the user 120 to perform visual activities under various simulated corrective measures, such as different types of lenses or vision correction techniques. The eye-tracking sensors may monitor the user's gaze direction, fixation duration, and response accuracy, while the visual assessment application 328 adjusts the visual simulations in real time to reflect different corrective measures, thereby enabling comprehensive evaluation of effectiveness of the corrective measures.

In some embodiments, the VR-based computer system 300 may incorporate a range of interactive tasks, such as reading text at varying distances, identifying objects in low light, and navigating through dynamic virtual environments. These tasks are configured to test the user's visual acuity, depth perception, and overall visual comfort under each simulated correction scenario. A user application 324 (e.g., visual assessment application 328 in FIG. 3) may process the data and evaluate parameters, such as clarity, sharpness, and user satisfaction with each correction type. The computer system 300 may determine effective corrective measures customized based on the user's specific visual needs. Results may be compiled into a report that provides insights into the user's visual performance and the recommended vision correction solution. As such, the computer system 300 may offer a dynamic, engaging, and precise approach to prescribing corrective measures, representing a significant advancement over traditional static vision tests.

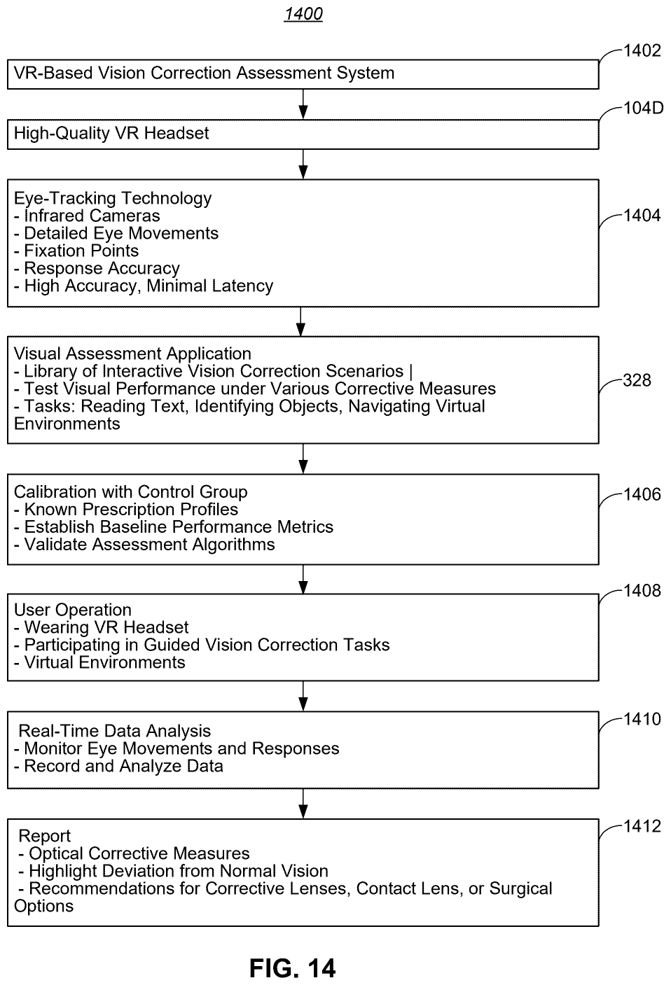

FIG. 14 is a flow diagram of an example vision test process 1400 for determining corrective measures based on vision correction simulation, in accordance with some embodiments. The VR-based computer system 300 may be configured to enable a VR-based vision correction assessment system 1402. The computer system 300 may include a VR headset 104D that includes an eye-tracking camera 366 (FIG. 3). The eye-tracking technology may include an infrared camera (e.g., camera 366) configured to capture (operation 1404) eye movements, fixation points, and response accuracy with high accuracy and minimal latency. In some embodiments, when a visual assessment application 328 is executed, a library of interactive cognitive tasks may be applied to test different aspects of visual performance under various corrective measures. These scenarios include tasks where the user may be prompted to read text at different distances, identify objects under various lighting conditions, and navigate through complex virtual environments.

In some embodiments, when hardware components and software modules may be integrated to form the VR-based vision correction assessment system 1402, the VR-based computer system 300 may be calibrated (operation 1406) using a control group of individuals with known vision correction profiles to establish baseline performance metrics and validate the accuracy of the assessment algorithms. Users can operate (operation 1408) the calibrated computer system 300 by wearing the VR headset and participating in the guided vision correction tasks within the virtual environments. The eye-tracking camera 366 may monitor their eye movements and responses to the simulated corrective measures. Image or video data recorded by the camera 366 may be analyzed (operation 1410) in real time by the software modules (e.g., visual assessment application 328, data processing module 330 in FIG. 3). In some implementations, the user may receive a report 1412 outlining the optimal corrective measures, highlighting any deviations from normal vision, and providing recommendations for corrective lenses, contact lenses, or surgical options. By these means, the computer system 300 may offer a precise, non-invasive, and user-friendly method for prescribing vision corrective measures, providing substantial benefits for both clinical applications and personal eye care routines.

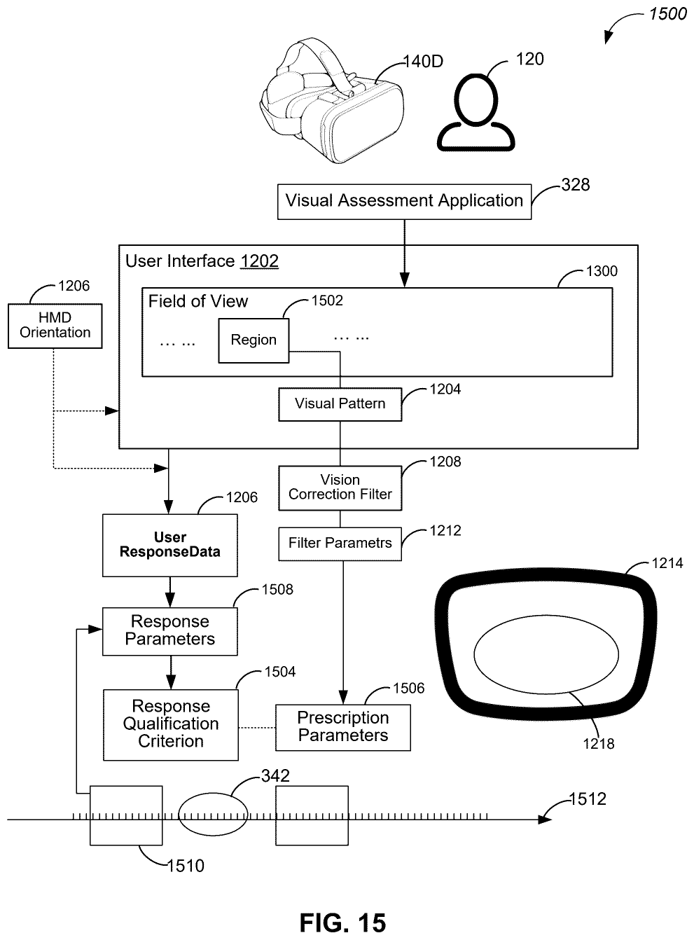

FIG. 15 is a flow diagram of an example vision test process 1500 for simulating vision correction, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. In some embodiments, a user 120 may face a field of view 1300 (FIG. 13) in the 3D virtual environment, and the field of view 1300 displayed on the user interface 1202 may include one or more regions 1502. The computer device 140 may render a visual pattern 1204 in the field of view 1300. A vision correction filter 1208 may be applied to process the visual pattern 1204. The computer device 140 may obtain a set of user response data 1206 captured by a plurality of sensors 360 in response to the visual pattern 1204 and determine whether the set of user response data 1206 satisfy a response quality criterion 1504. The computer device 140 may dynamically adjust filter parameters 1212 of the vision correction filter 1208 based on the set of user response data 1206, until the set of user response data 1206 satisfy the response quality criterion 1504.

In some embodiments, the visual pattern 1204 is rendered in one of a plurality of regions 1502 of the field of view 1300, and the vision correction filter 1208 may mimic a lens portion 1218 of an eyewear lens 1214 for improving perception of the visual pattern 1204 in the one of the plurality of regions 1502 of the field of view 1300. Further, In some embodiments, in accordance with a determination that the set of user response data 1206 satisfy the response quality criterion 1504, the computer device 140 may convert the filter parameters 1212 of the vision correction filter to a set of one or more eye prescription parameters 1506 associated with the lens portion 1218 of the eyewear lens 1214 for the user 120. Further, in some embodiments, the computer device 140 may identify a selection of the eyewear lens 1214. Based on the selection of the eyewear lens 1214, the computer device 140 may identify the lens portion on the eyewear lens 1214. In some embodiments, the eyewear lens 1214 may vary in size and shape, and the lens portion 1218 may be adaptively determined for the selection of the eyewear lens 1214.

Additionally, in some embodiments, the set of one or more eye prescription parameters 1506 associated with the lens portion 1218 of the eyewear lens 1214 includes one or more of: Sphere, Cylinder, Axis, ADD, PD, Prism, and Base, indicating the correction needed for vision. OD (oculus dexter) refers to the right eye, and OS (oculus sinister) refers to the left eye. Each eye will have its own set of numbers. Sphere measures the degree of nearsightedness (negative value) or farsightedness (positive value). Cylinder indicates the degree of astigmatism, reflecting the irregular shape of the cornea; a negative number means correction is required. Axis is given in degrees (from 0 to 180) and specifies the orientation of the astigmatism. For those with presbyopia or needing bifocals, ADD refers to the additional magnifying power for reading. PD (pupillary distance) may be listed to measure the distance between the pupils, ensuring proper lens alignment. Optional parameters may include prism (for correcting double vision) and base, which shows the direction of the prism correction.

In some embodiments, the computer device 140 may determine whether the HMD 312A is oriented forward. The visual pattern 1204 may be rendered and the set of user response data 1206 may be processed in accordance with a determination that the HMD 312A is oriented forward.

In some embodiments, when the computer device 140 determines whether the set of user response data 1206 satisfy the response quality criterion 1504, it may determine a plurality of response parameters 1508 based on the set of response data 1206. A combination of the plurality of response parameters 1508 may be determined. The computer device 140 may further determine whether the combination of the plurality of response parameters 1508 satisfies the response quality criterion 1504. The combination of the plurality of response parameters 1508 may indicate that the user 120 is comfortable with the visual pattern 1204 that has been corrected by the vision correction filter 1208. When this occurs, the computer device 140 may finalize the vision correction filter 1208 and apply the associated filter parameter 1212 to determine the prescription parameters 1506, e.g., automatically and without user intervention.

In some embodiments, the set of response data 1206 are captured in a temporal window 1510, and each of the plurality of sensors 360 (FIG. 3) has a respective sampling rate and may provide a subset of response data items (e.g., corresponding to sensor data 342 in FIG. 3) based on the respective sampling rate. The temporal window 1510 may move along a time axis 1512. Further, in some embodiments, the plurality of response parameters 1508 may be determined based on the temporal window 1510, and include one or more of: an eye blinking rate, a gaze direction, a fixation duration, a stress level, a focus level, a response time, a response accuracy level, and a micro expression type. For example, a focus level value may be determined for each temporal window 1510 based on the subset of response data items included within the temporal window 1510.

FIG. 16 is a flow diagram of an example response processing method 1600 for determining response parameters 1508, in accordance with some embodiments. Machine learning is applied to determine whether the user 120 is comfortable with the visual pattern 1204 that has been corrected by the vision correction filter 1208. In some embodiments, for each of the plurality of sensors 360, the computer device 140 may apply a sensor feature extraction model 1602 to process a subset of response data and generate a respective sensor feature vector 1604. A response monitoring model 1606 may be applied to process respective sensor feature vectors 1604 of the plurality of sensors 360 and generate a response quality indicator 1608 indicating whether the set of user response data 1206 satisfy the response quality criterion 1504. Stated another way, the response quality indicator 1608 may quantitatively indicate a comfort level of the user 120 with the visual pattern 1204 that has been corrected by the vision correction filter 1208. Further, in some embodiments, the response monitoring model 1606 may process a visual pattern identification 1610, display parameters 1612 of the visual pattern 1204, and a location 1614 of the visual pattern 1204 jointly with the respective sensor feature vectors 1604.

In some embodiments, a server 102 may collect training data from a plurality of eye patients. The training data may include response data 1206 of the plurality of eye patients associated with the visual pattern 1204 and train the response monitoring model 1606 based on the training data. The server 102 may provide the response monitoring model 1606 that has been trained to the computer device 140.

In some embodiments, the plurality of sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. The computer device 140 may render a visual pattern 1204 in the field of view. A vision correction filter may be applied to process the visual pattern 1204. The computer device 140 may obtain a set of user response data captured by a plurality of sensors in response to the visual pattern 1204. Filter parameters of the vision correction filter may be adjusted based on the set of user response data. In accordance with a determination that the set of user response data satisfy a response quality criterion, the computer device 140 may generate a prescription of an eyewear 1214 (e.g., including prescription parameters 1506) based on the filter parameters 1212 of the vision correction filter 1208.

In some embodiments, the visual pattern 1204 is rendered in at least one of a plurality of regions 1502 of the field of view 1300, and the vision correction filter 1208 is configured to mimic a lens portion 1218 of an eyewear lens 1214 for improving perception of the visual pattern 1204 in the one of the plurality of regions 1502 of the field of view 1300.

In some embodiments, the visual pattern 1204 covers a plurality of regions of 1502 the field of view 1300, and each region 1502 corresponds to a subset of respective filter parameters 1212. The computer device 140 may adjust the filter parameters 1212 of the vision correction filter 1208 by adjusting the subset of respective filter parameters 1212 for at least one region 1502 based on the set of user response data 1206 during each of a plurality of iteration.

### Determination of Visual Susceptibility to Digital Screen Use

Some implementations of this application include a VR-based computer system 300 configured to evaluate the effects of digital screen exposure on vision health. The computer system 300 may include a VR headset 104D that includes an eye-tracking camera 366 (FIG. 3). The eye-tracking camera 366 may include an infrared camera configured to capture eye movements and fixation patterns with high accuracy and minimal latency. In some embodiments, a visual assessment application 328 may be executed to generate simulations of prolonged digital screen use in various environmental settings. A user 120 may wear the VR headset and perform a series of tasks that may replicate typical screen-based activities, such as reading, gaming, and working on digital documents. In some embodiments, the eye-tracking camera 366 may monitor the user's gaze direction, blink rate, and fixation duration, while the visual assessment application 328 analyzes these responses to assess the impact of extended screen exposure on visual parameters such as eye strain, focus fatigue, and blinking patterns.

In some embodiments, the VR-based computer system 300 may incorporate a range of scenarios that mimic real-world digital screen usage, exposing users to different screen types, brightness levels, text sizes, and background lighting conditions. Tasks are configured to challenge the visual system by requiring prolonged focus, rapid eye movements, and frequent shifts between near and far visual targets. A user application 324 (e.g., visual assessment application 328 in FIG. 3) may process the data and evaluate parameters, such as changes in blink rate, incidence of dry eye symptoms, and visual discomfort over time. Results may be compiled into a report that provides detailed insights into the user's vision health, identifying specific symptoms of digital eye strain and offering recommendations for mitigating these effects, such as screen time management, optimal lighting conditions, and the use of blue light filters or corrective lenses. As such, the computer system 300 may offer a dynamic, engaging, and precise approach to understanding and managing the impact of digital screen exposure on vision health, representing a significant advancement over traditional eye health assessments.

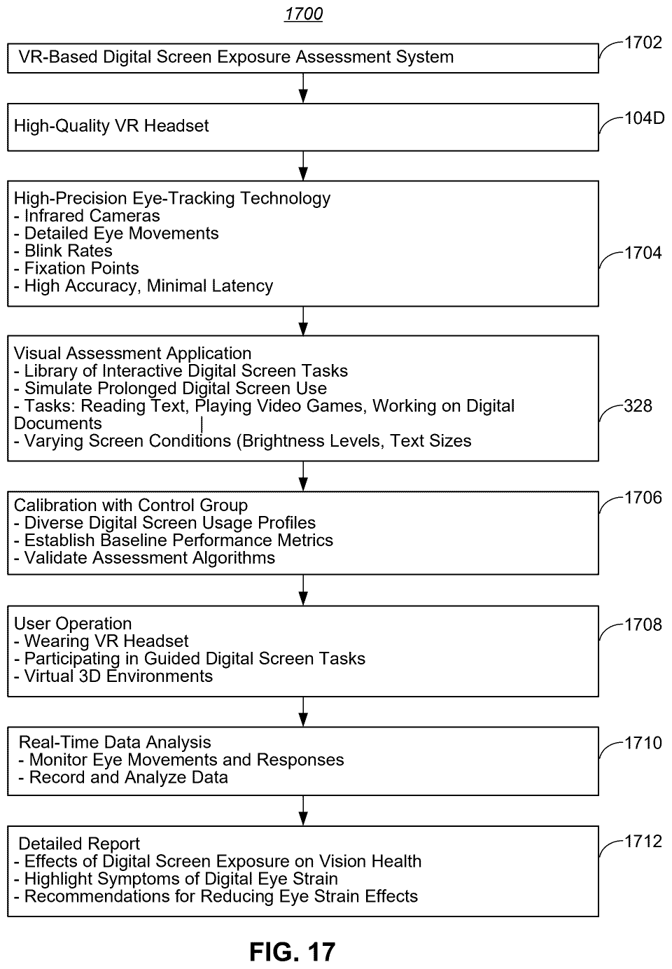

FIG. 17 is a flow diagram of an example vision test process 1700 for evaluating visual susceptibility of a user's eyes to digital screen exposure, in accordance with some embodiments. The VR-based computer system 300 may be configured to enable a VR-based digital screen exposure assessment system 1702. The computer system 300 may include a VR headset 104D that includes an eye-tracking camera 366 (FIG. 3). The eye-tracking technology may include an infrared camera (e.g., camera 366) configured to capture (operation 1704) eye movements, blink rates, and fixation patterns with high accuracy and minimal latency. In some embodiments, when a visual assessment application 328 is executed, a library of interactive cognitive tasks may be applied to simulate prolonged digital screen use. These tasks include scenarios where the user may be prompted to read text, play video games, and work on digital documents under varying screen conditions, such as different brightness levels and text sizes.

In some embodiments, when hardware components and software modules may be integrated to form the VR-based digital screen exposure assessment system 1702, the VR-based computer system 300 may be calibrated (operation 1706) using a control group of individuals with diverse digital screen usage profiles to establish baseline performance metrics and validate the accuracy of the assessment algorithms. Users can operate (operation 1708) the calibrated computer system 300 by wearing the VR headset and participating in the digital screen tasks within the virtual environments. The eye-tracking camera 366 may monitor their eye movements and responses to the screen exposure. Image or video data recorded by the camera 366 may be analyzed (operation 1710) in real time by the software modules (e.g., visual assessment application 328, data processing module 330 in FIG. 3). In some implementations, the user may receive a report 1712 outlining the effects of digital screen exposure on their vision health, highlighting any symptoms of digital eye strain and providing recommendations for reducing these effects. By these means, the computer system 300 may offer a precise, non-invasive, and user-friendly method for evaluating and managing the impact of digital screen exposure on vision health, providing substantial benefits for both clinical applications and personal eye care routines.

FIG. 18 is a flow diagram of an example vision test process 1800 for assessing digital screen use by a user 120, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1802 corresponding to a 3D virtual environment. The computer device 140 may display visual content 1804 continuously for an extended duration of time 1806 in the 3D virtual environment, and the visual content 1804 may be displayed with predefined display parameters 1808 associated with a screen usage. The computer device 140 may obtain a stream of sensor data 1810 measured by the one or more sensors 360 and determine a plurality of sequential user responses 1812 to the visual content 1804 based on the stream of sensor data 1810. The computer device 140 may apply at least a screen usage prediction model 1814 to generate a screen usage guidance profile 1816 for the user 120 based on the plurality of sequential user responses 1812.

In some embodiments, the screen usage guidance profile 1816 may include one or more: a screen size 1816A, a color scheme 1816B, a font size 1816C, a screen angle 1816D, a screen height 1816E, a background lighting condition 1816F, and a screen use time limit 1816G. In some embodiments, the screen usage guidance profile 1816 may include a brightness level 1816I and a contrast level 1816J of a screen to be used by the user 120. In some embodiments, the screen usage guidance profile 1816 requires that the brightness level 1816H of the screen varies with a time spent on the screen, and the computer device 140 may automatically adjust the brightness level 1816H of the screen based on the screen usage guidance profile 1816. In some embodiments, the screen usage guidance profile 1816 requires that the contrast level 1816J of the screen varies with a time spent on the screen, and the computer device 149 may automatically adjust the contrast level of the screen based on the screen usage guidance profile 1816.

In some embodiments, the screen usage guidance profile 1816 includes a color scheme 1816B. The computer device 140 may apply a first color scheme to a user interface 1802 associated with a user application 324 (e.g., a media play application). In accordance with a determination that a time spent on a screen by the user 120 is greater than a screen time limit, the computer device 140 may apply a second color scheme to the user interface 1820 associated with the user application 324.

In some embodiments, the plurality of sensors 360 may include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. In some embodiments, the plurality of sequential user responses 1812 include one or more of: an eye blinking rate, a gaze direction, a fixation duration, a stress level, a focus level, a fatigue level, a response time, a response accuracy level, and a micro expression type. For example, the plurality of sequential user responses 1812 may be determined based on eye images captured by the eye-tracking camera 366, such that, the screen usage guidance profile 1816 may be determined automatically based on the plurality of sequential user responses 1812 without user intervention.

In some embodiments, the stream of sensor data 1810 may be captured according to a temporal window 1510, and each of the one or more sensors may have a respective sampling rate and provide a subset of sensor data 342 based on the respective sampling rate. The temporal window 1510 may move along a time axis 1512. Further, in some embodiments, for each of the one or more sensors 360, the computer device 140 may apply a sensor feature extraction model 1822 to process the subset of sensor data 1810 and generate a respective sensor feature vector 1824. A response monitoring model 1826 may be applied to process respective sensor feature vectors 1824 of the one or more sensors 360 and generate a respective sequential user response 1812 corresponding to the temporal window 1510.

In some embodiments, the predefined display parameters 1808 include a plurality of corner display parameters each of which is substantially close to a respective display parameter limit. For example, the brightness level and the contrast level of the predefined display parameters 1808 much are higher than normal brightness and contrast levels used by the user. Based on the user's response, the computer device 140 may expedite the vision test to predict the user's response to an elevated brightness or contrast level, thereby suggesting the brightness level 1816H and the contrast level 1816J in the screen usage guidance profile.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. The computer device 140 may display visual content 1804 continuously for an extended duration of time 1806 in the 3D virtual environment, and the visual content 1804 may be displayed with predefined display parameters 1808 associated with a screen usage. The computer device 140 may obtain a stream of sensor data 1810, determine a plurality of sequential user responses 1812 to the visual content 1804 based on the stream of sensor data 1810, and generate a screen usage guidance profile 1816 for the user 120 based on the plurality of sequential user responses 1812. The screen usage guidance profile 1816 may include at least a time-dependent display parameter 1818 (e.g., a color scheme 1816B or a contrast level 1816J adjustable based on a screen use time).

In some embodiments, the time-dependent display parameter 1818 may include one or more of: a color scheme 1816B, a font size 1816C, a background lighting condition 1816F, a contrast level 1816J, and a brightness level 1816I. In some embodiments, the time-dependent display parameter 1808 may include least two settings of: a color scheme 1816B, a font size 1816C, a background lighting condition 1816F, a contrast level 1816J, and a brightness level 1816I, and the two settings have temporal dependences that are independent of one another.

### Streamlined and Automatic Evaluation of Vision Changes

Some implementations of this application include a VR-based computer system 300 configured to evaluate the effectiveness of prescription changes in eyeglasses or contact lenses by comparing before-and-after vision test results. The computer system 300 may include a VR headset 104D that includes an eye-tracking camera 366 (FIG. 3). The eye-tracking camera 366 may include an infrared camera configured to capture eye movements and fixation patterns with high accuracy and minimal latency. In some embodiments, a visual assessment application 328 may be executed to generate standardized vision tests in a controlled virtual environment. A user 120 may wear the VR headset and perform a series of vision tests both before and after receiving new prescription lenses. In some embodiments, the eye-tracking camera 366 may monitor the user's gaze direction, fixation stability, and response times, while the visual assessment application 328 analyzes these responses to provide detailed assessment of visual performance under each prescription.

In some embodiments, the VR-based computer system 300 may incorporate a range of vision tests such as reading eye charts at varying distances, identifying symbols and objects, and performing tasks that require depth perception and visual acuity. The tests are configured to be interactive and engaging, ensuring accurate and consistent user responses. A user application 324 (e.g., visual assessment application 328 in FIG. 3) may process the data and evaluate parameters, such as visual clarity, sharpness, and focus stability. When test results from the pre- and post-prescription assessments are compared, results may be compiled into a report that highlights improvements or persistent issues in visual performance. As such, the computer system 300 may offer a dynamic, precise, and user-friendly approach to evaluating the effectiveness of prescription changes, representing a significant advancement over traditional static vision tests.

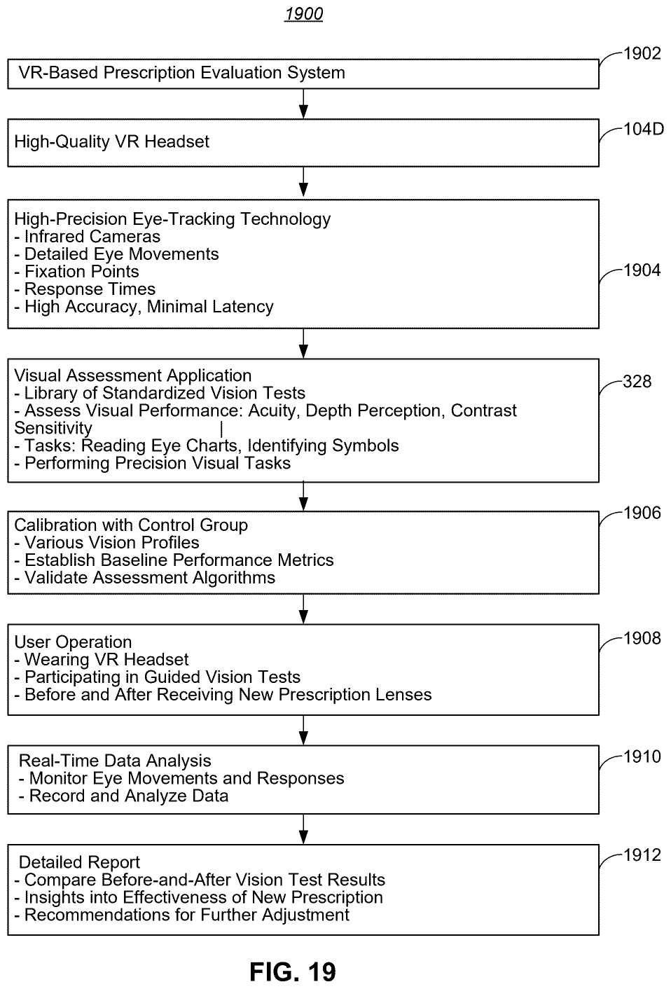

FIG. 19 is a flow diagram of an example vision test process 1900 for evaluating prescription changes in a 3D virtual environment, in accordance with some embodiments. The VR-based computer system 300 may be configured to enable a VR-based prescription evaluation system 1902. The computer system 300 may include a VR headset 104D that includes an eye-tracking camera 366 (FIG. 3). The eye-tracking technology may include an infrared camera (e.g., camera 366) configured to capture (operation 1904) eye movements, fixation points, and response times with high accuracy and minimal latency. In some embodiments, when a visual assessment application 328 is executed, a library of interactive cognitive tasks may be applied to assess different aspects of visual performance, such as acuity, depth perception, and contrast sensitivity. These tests include scenarios where the user may be prompted to read eye charts, identify symbols, and perform tasks requiring precise visual discrimination within the virtual environment.

In some embodiments, when hardware components and software modules may be integrated to form the VR-based prescription evaluation system 1902, the VR-based computer system 300 may be calibrated (operation 1906) using a control group of individuals with various vision profiles to establish baseline performance metrics and validate the accuracy of the assessment algorithms. Users can operate (operation 1908) the calibrated computer system 300 by wearing the VR headset and participating in the guided vision tests before and after receiving new prescription lenses. The eye-tracking camera 366 may monitor their eye movements and responses during the tests. Image or video data recorded by the camera 366 may be analyzed (operation 1910) in real time by the software modules (e.g., visual assessment application 328, data processing module 330 in FIG. 3). In some implementations, the user may receive a report 1912 comparing the before-and-after vision test results, providing insights into the effectiveness of the new prescription and offering recommendations for further adjustments if necessary. By these means, the computer system 300 may offer a precise, non-invasive, and user-friendly method for evaluating prescription changes, providing substantial benefits for both clinical applications and personal eye care routines.

FIG. 20 is a flow diagram of an example vision test process 2000 for tracking a chronic eye condition, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 2002 corresponding to a 3D virtual environment. The computer device 140 may display a predefined video clip 2004 in the 3D virtual environment, and the predefined video clip 2004 may include a plurality of visual sessions corresponding to a sequence of vision tests 2006. While the predefined video clip 2004 is played, the computer device 140 may obtain a stream of sensor data 342 measured by the one or more sensors 360 (FIG. 3) and determine a plurality of first response parameters 2008 to the sequence of vision tests 2006 based on the stream of sensor data 342.

In some embodiments, for each of one or more subsequent iterations 2040, the computer device 140 may repeat display of the predefined video clip 2004 in the 3D virtual environment and determine a plurality of second response parameters 2010. The one or more subsequent iterations 2040 may be implemented a number of days, months, or years after the first response parameters 2008 are determined, thereby tracking response parameters of the user's eyes chronically. That said, in some embodiments, the computer device 140 may track a variation of response parameters 2012 based on the plurality of first response parameters 2008 and the plurality of second response parameters 2010 of each subsequent iteration 2040. Additionally, in some embodiments, each subsequent iteration 2040 is implemented on a distinct day, and the variation of response parameters 2012 may indicate chronic development of the user's eyesight. Further, in some embodiments, the computer device 140 may apply a chronic development model 2014 to process the plurality of first response parameters 2008 and the plurality of second response parameters 2010 of each subsequent iteration 2040 jointly and generate a chronic condition output 2016 associated with the variation of response parameters. For example, the chronic condition output 2016 includes one or more of: an eyesight drop trend, an eyesight drop rate 2016A, whether each of a plurality of known eye conditions 2016B newly occurs, whether each of a plurality of existing eye conditions 2016C gets worse or better, and whether further professional consultation 2016D is needed.

In some embodiments, the predefined video clip 2004 may be displayed while a user 120 associated with the computer device 140 is wearing an eyewear having a first eyewear prescription 2018, and the plurality of first response parameters 2008 correspond to the eyewear having the first eyewear prescription 2018. Further, in some embodiments, while the user 120 is wearing an eyewear having a second eyewear prescription 2020, the computer device 140 may repeat display of the predefined video clip in the 3D virtual environment, determine a plurality of second response parameters 2010, and compare the plurality of first response parameters 2008 and the plurality of second response parameters 2010. Based on a comparison result, the computer device 140 may determine whether the second eyewear prescription 2020 improves eyesight correction compared with the first eyewear prescription 2018.

In some embodiments, the plurality of sensors 360 include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. In some embodiments, the plurality of first response parameters 2008 may include one or more of: an eye blinking rate, a gaze direction, a fixation duration, a stress level, a focus level, a fatigue level, a response time, a response accuracy level, and a micro expression type. In other words, in some embodiments, the plurality of first response parameters 2008 correspond to a spontaneous user response that is tracked automatically without user intervention. For example, the first response parameters 2008 may be determined based on eye images captured by the eye-tracking camera 366. In an example, if the response time of the user's eyes is shortened or the stress level of the user's eyes is lower when the second prescription 2020, the second prescription 2020 may be determined as enhancing eyesight correction compared with the first prescription 2018.

FIG. 21A is a flow diagram of an example response processing method 2100 for determining response parameters 2008 or 2010, in accordance with some embodiments. In some embodiments, the stream of sensor data 342 may be captured according to a temporal window 1510, and each of the one or more sensors 360 has a respective sampling rate and provides a subset of sensor data 342 based on the respective sampling rate. The temporal window 1510 may move along a time axis 1512. Further, in some embodiments, for each of the one or more sensors 360, the computer device 140 may apply a sensor feature extraction model 2102 to process the subset of response data 342 of a respective sensor 360 and generate a respective sensor feature vector 2104 (e.g., corresponding to one of the temporal windows 1510). A response monitoring model 2106 may be applied to process respective sensor feature vectors 2104 of the one or more sensors 360 and generate a respective sequential user response 2108 corresponding to the temporal window 1510. Respective sequential user responses 2108 of a set of successive temporal windows 1510 may be further combined to determine the plurality of first response parameters 2008 or 2010.

In some embodiments, a server 102 may collect training data from a plurality of eye patients. The training data may include sensor data 342 of the plurality of eye patients associated with the video clip 2004 and train the response monitoring model 2106 based on the training data. The server 102 may provide the response monitoring model 2106 that has been trained to the computer device 140.

FIG. 21B is a flow diagram of an example response processing method 2150 for determining a chronic vision change 2160, in accordance with some embodiments. Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. The computer device 140 may display a predefined video clip 2004 in the 3D virtual environment, and the predefined video clip 2004 may include a plurality of visual sessions corresponding to a sequence of vision tests 2006. While the predefined video clip 2004 is played, the computer device 140 may obtain a stream of sensor data 342 measured by the one or more sensors 360 (FIG. 3) and determine a current response feature vector indicating a user response to the sequence of vision tests based on the stream of sensor data 342. The computer device 140 may determine a chronic vision change 2160 of a user 120 associated with the computer device 140 based on a plurality of response feature vectors including a current response feature vector 2152.

In some embodiments, the plurality of response feature vectors may further include a set of one or more historical response feature vectors 2154. The computer device 140 may extract the set of one or more historical response feature vectors 2154 and apply a vision change model 2156 to process the plurality of response feature vectors 2152 and 2154 to determine the chronic vision change 2160 (e.g., a vision change trend). Stated another way, in some embodiments, the sensor data 342 or the response parameters 2008 and 2010 may not need to be stored. Eye performance may be compressed and tracked in the plurality of response feature vectors (e.g., current response feature vectors 2152, historical response feature vectors 2154), while each response feature vector may not define a response parameter (e.g., a response rate) directly.

### Determination of Sightline-Based Corrective Measures of Eyewear

Some implementations of this application include a VR-based computer system 300 configured to determine optimal vision correction parameters through a method of successive approximation. The computer system 300 may include a VR headset 104D that includes an eye-tracking camera 366 (FIG. 3). The eye-tracking camera 366 may include an infrared camera configured to capture eye movements and fixation patterns with high accuracy and minimal latency. In some embodiments, a visual assessment application 328 may be executed to generate a series of vision correction simulations. A user 120 may wear the VR headset and perform a series of tasks that simulate different vision correction parameters, such as varying lens strengths and focal adjustments. In some embodiments, the eye-tracking camera 366 may monitor the user's gaze direction, fixation stability, and visual response accuracy, while the visual assessment application 328 dynamically adjusts the vision correction parameters based on the user's real-time feedback and performance, using a method of successive approximation to enhance correction settings.

In some embodiments, the VR-based computer system 300 may incorporate a range of tasks configured to test visual acuity, depth perception, and focus stability, such as reading text at different distances, identifying symbols, and performing tasks that require precise visual discrimination. A user application 324 (e.g., visual assessment application 328 in FIG. 3) may process the data and evaluate parameters, such as clarity, sharpness, and user comfort with each simulated correction setting. Based on the user's responses and performance, the computer system 300 may iteratively adjust the vision correction parameters, gradually refining the settings to achieve the best possible visual outcome. Results may be compiled into a report that provides detailed insights into the user's optimal vision correction parameters, offering precise recommendations for eyeglass or contact lens prescriptions. As such, the computer system 300 may offer a dynamic, engaging, and highly accurate approach to determining vision correction needs, representing a significant advancement over traditional static vision tests.

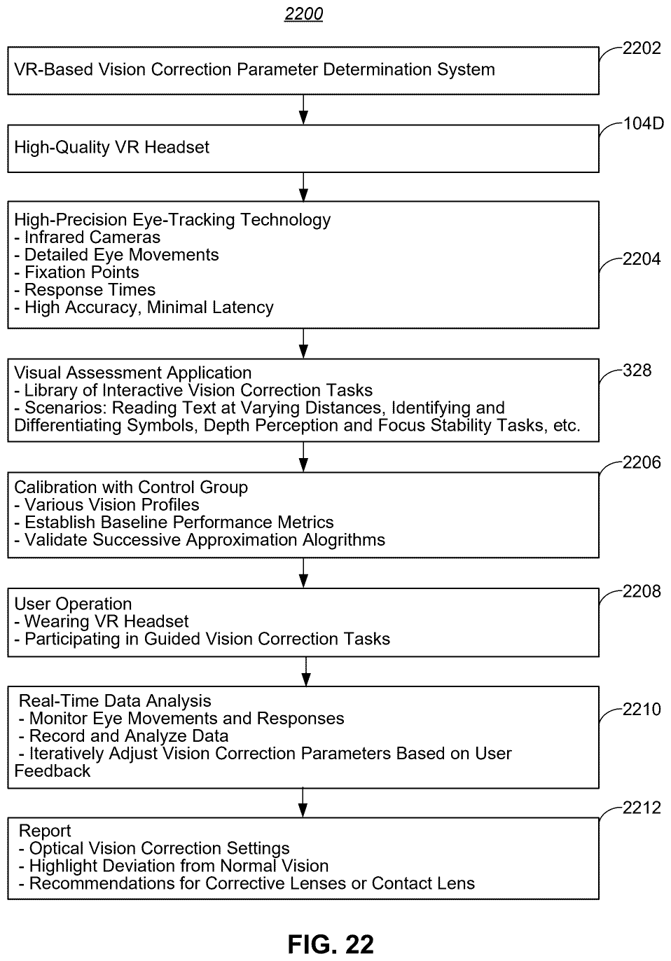

FIG. 22 is a flow diagram of an example vision test process 2200 for determining optimal vision correction parameters through successive approximation, in accordance with some embodiments. The VR-based computer system 300 may be configured to enable a VR-based vision correction parameter determination system 2202. The computer system 300 may include a VR headset 104D that includes an eye-tracking camera 366 (FIG. 3). The eye-tracking technology may include an infrared camera (e.g., camera 366) configured to capture (operation 2204) eye movements, fixation points, and response times with high accuracy and minimal latency. In some embodiments, when a visual assessment application 328 is executed, a library of interactive cognitive tasks may be applied to test various aspects of visual performance under different vision correction simulations. These tasks include scenarios where the user may be prompted to read text at varying distances, identify and differentiate symbols, and perform tasks that require accurate depth perception and focus stability.

In some embodiments, when hardware components and software modules may be integrated to form the VR-based vision correction parameter determination system 2202, the VR-based computer system 300 may be calibrated (operation 2206) using a control group of individuals with known vision profiles to establish baseline performance metrics and validate the accuracy of the successive approximation algorithms. Users can operate (operation 2208) the calibrated computer system 300 by wearing the VR headset and participating in the guided vision correction tasks within the virtual environments. The eye-tracking camera 366 may monitor their eye movements and responses to the dynamic correction simulations. The computer system 300 may iteratively adjust the vision correction parameters based on user feedback, refining the settings to determine the optimal correction parameters. Image or video data recorded by the camera 366 may be analyzed (operation 2210) in real time by the software modules (e.g., visual assessment application 328, data processing module 330 in FIG. 3). In some implementations, the user may receive a report 2212 outlining their optimal vision correction settings, highlighting any deviations from normal vision, and providing precise recommendations for corrective lenses or contact lenses. By these means, the computer system 300 may offer a precise, non-invasive, and user-friendly method for determining vision correction parameters, providing substantial benefits for both clinical applications and personal eye care routines.

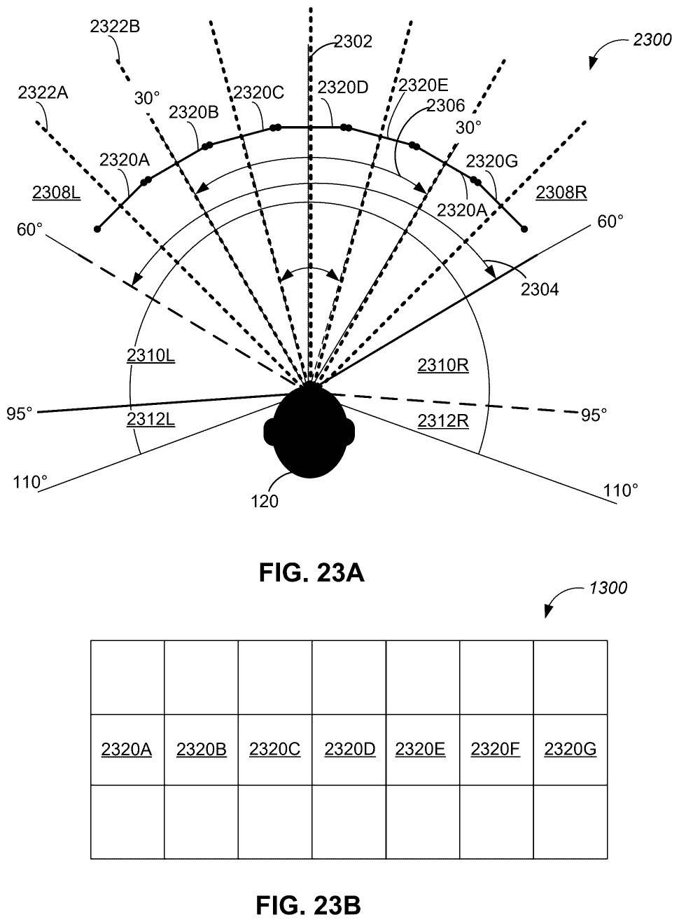

FIG. 23A is a diagram of an example horizontal field of view (HFOV) 2300 of a user's eyes, in accordance with some embodiments, and FIG. 23B is a schematic diagram of an example field of view 1300 including three rows of regions corresponding to a plurality of lines of sight, in accordance with some embodiments. The HFOV 2300 refers to the extent of a visual field that the user 120 can see from side to side, measured in degrees. The HFOV 2300 may include a monocular view of each eye of the user 120 referring to a portion of the HFOV 2300 perceived by the respective eye at a time. In some embodiments, for a single eye, the HFOV 2300 is typically around 155 degrees, depending on the user's eye anatomy. For example, a reference axis 2302 extends forward from a middle point of a line connecting the user's two eyes. A left monocular view of a left eye covers an angular range from −95° to +60° with respect to the reference axis 2302, and a right monocular view of a right eye covers an angular range from −60° to +95° with respect to the reference axis 2302. If only one eye is used, depth perception may be limited, and an object may appear flatter compared to when use both eyes.

The left monocular view of the left eye and the right monocular view of the right eye may overlap in a binocular area 2304 covering a binocular angular range (e.g., [−60°, 60°]). The binocular area 2304 occurs when both eyes work together, allowing for depth perception and a more accurate representation of 3D space. The binocular area 2304 is where stereoscopic vision occurs, providing depth and spatial awareness. Stereoscopic vision is the ability to perceive depth and three-dimensional structure by integrating visual information from both eyes. Each eye captures a slightly different image because they are spaced apart (about 6-7 cm in humans), giving each eye a unique angle on the same object. The user's brain processes and merges these two images associated with two eyes to create a single 3D perception, which is a process known as binocular fusion.

The binocular area 2304 may include an area of focus 2306 (e.g., from −30° to 30°, a left peripheral area 2308L (e.g., between 30 and 60 degrees to the left of the reference axis 2302), and a right peripheral area 2308R (e.g., between 30 and 60 degrees to the right of the reference axis 2302). For example, the peripheral area 2308L or 2308R is about 30 degrees. The HFOV 2300 further includes a left edge area 2310L that is only visible to the left eye and a right edge area 2310R that is only visible to the right eye. The left edge area 2310L and the right edge area 2310R are immediately adjacent to the binocular area 2304. Each of the edge areas 2310L and 2310R may cover an angular range of 35°. Additionally, the HFOV 2300 is further expanded by a temporal area 2312L or 2312R (e.g., corresponding to) 15° on each of two sides of the user's head. The binocular area 2304, the edge areas 2310L and 2310R, and the temporal area 2312L and 2312R contribute to the overall perception of the surrounding environment, with the binocular area 2304 providing enhanced depth and spatial information crucial for activities like driving, sports, or reading depth cues in daily life. In some situations, the user 120 has an impaired HFOV in which the binocular area 2304 covers less than a normal binocular angular range (e.g., the impaired HFOV spans from −40° to 60°, rather than from −60° to 60°). The user's depth perception is compromised within part of the normal binocular angular range.

Referring to FIG. 23B, in some embodiments, the field of view 1300 may include three rows of regions 2320 corresponding to a plurality of lines of sight. The three rows of regions 2320 may include a middle row of regions 2320A to 2320G associated with the HFOV 2300. When the user 120 faces forward without lifting up or lowering down his head, the middle row of regions 2320A to 2320G are located at a height of the user's eyes. The middle row of regions 2320A to 2320G may correspond to a set of lines of sight, e.g., separated by 15 degrees from one another. For example, the region 2320A may correspond to a line of sight 2322A that may pass a center of the region 2320A, be 45 degrees to the left of the reference axis 2302, and have a spanning angle of 15 degrees, while the region 2320B may correspond to a line of sight 2322B that may pass a center of the region 2320B, be 30 degrees to the left of the reference axis 2302, and have a spanning angle of 15 degrees.

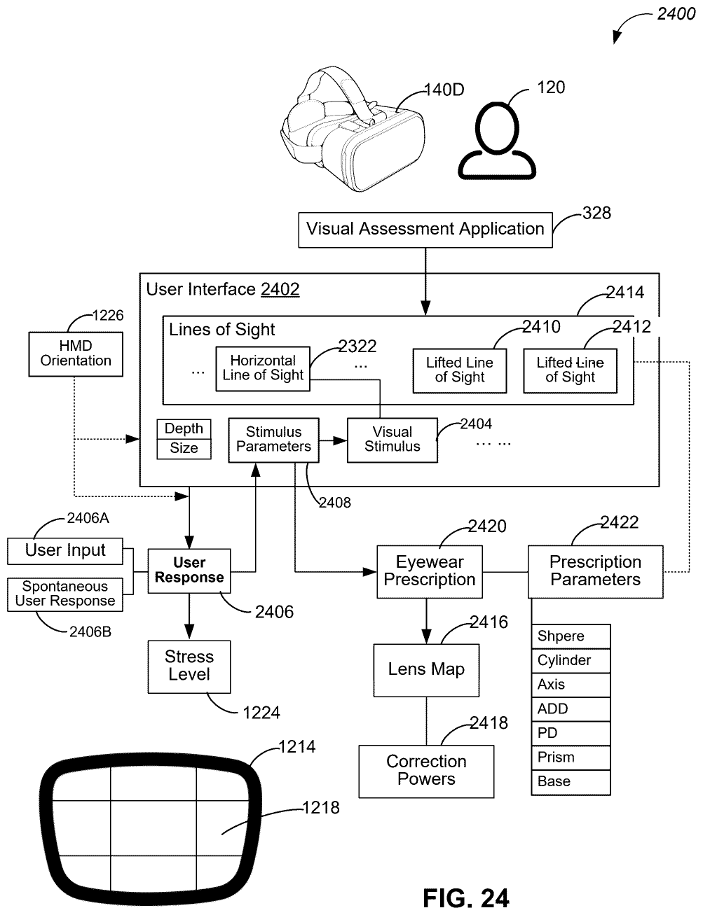

FIG. 24 is a flow diagram of an example vision test process 2400 for determining an eyewear prescription corresponding to a plurality of horizontal lines of sight, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 2402 corresponding to a 3D virtual environment. The computer device 140 may identify a plurality of horizontal lines of sight 2322 (e.g., lines 2322A and 2322B in FIG. 23). For each horizontal line of sight 2322, the computer device 140 may render a respective visual stimulus 2404 on the respective horizontal line of sight 2322, obtain a user response 2406 to the respective visual stimulus 2404, and dynamically adjust stimulus parameters 2408 of the respective visual stimulus 2404 based on the user response 2406. Based on the stimulus parameters 2408 associated with each horizontal line of sight 2322, the computer device 140 may determine an eyewear prescription 2420 of an eyewear for a user 120 associated with the computer device 140. The eyewear prescription 2420 may include prescription parameters 2422 (e.g., corrective measures) corresponding to the plurality of horizontal lines of sight 2322.

In some embodiments, the stimulus parameters 2408 may include at least a stimulus depth and a stimulus size, and the prescription parameters 2408 corresponding to each horizontal line of sight 2322 may be determined based on the stimulus parameters 2408 of the respective visual stimulus 2404 associated with the respective horizontal line of sight 2322. For example, when an optotype is displayed at the line of sight 2322A (FIG. 23), the depth and size of the optotype may determine an acuity level of the user's eye associated with the horizontal line of sight 2322, and corrective measures may be determined based on the acuity level for the horizontal line of sight 2322. Thus, the corrective measures of the eyewear prescription may be different for adjacent horizontal lines of sight 2322A and 2322B. Stated another way, an angular resolution of the eyewear prescription 2420 is controlled at 15 degrees for the horizontal lines of sight 2322. When a lens 1214 is made based on the prescription 2420, the correction powers of every 15 degrees for the horizontal lines of sight 2322 may be different. For example, two different lens portions 1218 corresponding to the adjacent horizontal lines of sight 2322A and 2322B may have correction powers of −2.25 and −2.75, respectively.

In some embodiments, every two immediately adjacent lines of sight of the plurality of horizontal lines of sight 2322 may be separated by 15-30 degrees, inclusively. For example, referring to FIGS. 23A and 23B, two immediately adjacent lines of sight 2322 are separated by 15 degrees.

In some embodiments, the eyewear prescription 2420 may further include prescription parameters 2422 corresponding to a plurality of lifted lines of sight 2410 or a plurality of lowered lines of sight 2412. Further, in some embodiments, for each of the plurality of lifted lines of sight 2410 or the plurality of lowered lines of sight 2412, the computer device 140 may dynamically adjust stimulus parameters 2408 of the respective visual stimulus 2404 based on a user response 2406 to a respective visual stimulus 2404 displayed on the respective line of sight 2410 or 2412. In some situation, the user 120 may tilt up his head from the HFOV 2300 by a lift angle (e.g., 15 degrees) to look forward along the plurality of lifted lines of sight 2410. In some situation, the user 120 may tilt down his head from the HFOV 2300 by a lowered angle (e.g., 15 degrees) to look forward along the plurality of lowered lines of sight 2412.

In some embodiments, the prescription 2420 may map a plurality of lines of sight 2414 including the plurality of horizontal lines of sight 2322 with respective prescription parameters 2422. Further, in some embodiments, the computer device 140 may identify a selection of an eyewear lens 1214 (e.g., having a shape and a size). Based on the selection of the eyewear lens 1214, the computer device 140 may convert the respective prescription parameters 2422 of the plurality of lines of sight 2414 to a lens map 2416, and the lens map 2416 may associate a plurality of lens portions 1218 of the eyewear lens 1214 with a plurality of correction powers (e.g., spherical powers). For example, for each lens portion 1218, the computer device 140 may identify a subset of lines of sight 2414 that passes the respective lens portion 1218 and determine a prescription parameter 2422 of the respective lens portion 1218 as a combination of the prescription parameters 2422 of the subset of lines of sight 2414.

Additionally, in some embodiments, when the computer device 140 converts the respective prescription parameters 2422 of the plurality of lines of sight 2414 to the lens map 2416, for each of the plurality of lines of sight 2414, the computer device 140 may identify a respective lens portion 1218 of the eyewear lens 1214, and determine a respective correction power 2418 for the respective lens portion 1218 based on the respective prescription parameters 2422 corresponding to the respective line of sight 2414. Further, in some situations, the eyewear lens 1214 may not be evenly divided to provide the plurality of lens portions 1218.

In some embodiments, a horizontal field of view may be divided substantially evenly to identify the plurality of horizontal lines of sight.

In some embodiments, the respective visual stimulus 2404 displayed on each horizontal line of sight 2322 (e.g., 2322A to 2322G) may include a predefined visual stimulus 2404. The predefined visual stimulus 2404 is repeatedly displayed along different horizontal lines of sight 2322. In some embodiments, respective visual stimuli 2404 of the plurality of horizontal lines of sight 2322 may be rendered successively to determine a respective subset of the eyewear prescription 2420 for each respective horizontal line of sight 2322. Further, in some embodiments, the respective visual stimuli 2404 may be rendered successively in the plurality of horizontal lines of sight 2322 according to a random order.

In some embodiments, for each of the horizontal lines of sight 2322, the computer device 140 may determine a stress level 1224 based on the user response 2406 to the respective visual stimulus 2404 displayed in the respective horizontal line of sight 2322. In accordance with a determination that the stress level 1224 satisfies a response criterion, the computer device 140 may associate the respective horizontal line of sight 2322 with the respective stimulus parameters 2408 of the visual stimulus 2404. Stated another way, when the stress level 1224 satisfies the response criterion, the user 120 may perceive the visual stimulus 2404 to a satisfactory level, and the respective stimulus parameter 2408 of the visual stimulus 2404 can be finalized and converted to the prescription parameters 2422 for the eyewear prescription 2420.

In some embodiments, the user response 2406 include a user input captured by a subset of one or more sensors of the computer device 140, and the one or more first sensors include a forward-facing camera for detecting a hand gesture, a microphone for collecting an audio response, and a controller for receiving a user physical force.

In some embodiments, the user response includes a spontaneous user response monitored by a subset of one or more second sensors of the computer device 140, and the one or more second sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362.

In some embodiments, the computer device 140 may determine whether the HMD 312A is oriented forward (e.g., having an HMD orientation 1226). The respective visual stimulus 2404 may be rendered and the user response 2406 may be obtained and processed in accordance with a determination that the HMD 312A is oriented forward.

In some embodiments, a first horizontal line of sight 2322A is immediately adjacent to a second horizontal line of sight 2322B. The computer device 140 may set initial parameters of the respective visual stimulus 2404 of the second horizontal line of sight 2322B based on at least the stimulus parameters 2408 determined for the respective visual stimulus 2404 of the first horizontal line of sight 2322A.

In some embodiments, the prescription parameters 2422 of each horizontal line of sight 2322 include one or more of: Sphere, Cylinder, Axis, ADD, PD, Prism, and Base.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 2702 corresponding to a 3D virtual environment. The computer device 140 may identify a plurality of horizontal lines of sight 2322 and render a visual stimulus 2404 successively on the plurality of horizontal lines of sight 2322. The computer device 140 may dynamically adjust stimulus parameters 2408 of the visual stimulus 2404 based on a spontaneous user response 2406B. Based on the stimulus parameters 2408, the computer device 140 may determine an eyewear prescription 2420 of an eyewear for a user 120 associated with the computer device 140. The eyewear prescription 2420 may include corrective measurements 2418 corresponding to the plurality of horizontal lines of sight 2322.

In some embodiments, the computer device 140 may obtain a plurality of eye images captured by an eye-tracking camera 366 and determine the spontaneous user response 2406B based on the plurality of eye images. In some embodiments, the spontaneous user response 2406B may include one or more of: an eye blinking rate, a gaze direction, a fixation duration, a stress level, a focus level 1224, a response time, a response accuracy level, and a micro expression type.

### Determination of Multifocal Parameters of Multifocal Eyewear

Some implementations of this application include a VR-based computer system 300 configured for prescribing and adjusting bifocal and multifocal lenses within a virtual setting. The computer system 300 may include a VR headset 104D that includes an eye-tracking camera 366 (FIG. 3). The eye-tracking camera 366 may include an infrared camera configured to capture eye movements and fixation patterns with high accuracy and minimal latency. In some embodiments, a visual assessment application 328 may be executed to simulate the visual effects of bifocal and multifocal lenses. A user 120 may wear the VR headset and perform a series of tasks that simulate everyday activities requiring both near and distance vision, such as reading, driving, and computer work. In some embodiments, the eye-tracking camera 366 may monitor the user's gaze direction, fixation stability, and visual response accuracy, while the visual assessment application 328 dynamically adjusts the lens simulations based on real-time user feedback and performance. This iterative process may calibrate the lens parameters to suit individual visual needs.

In some embodiments, the VR-based computer system 300 may incorporate a range of scenarios that replicate real-world tasks requiring bifocal and multifocal vision, such as reading text at different distances, transitioning focus between near and far objects, and navigating through dynamic environments. A user application 324 (e.g., visual assessment application 328 in FIG. 3) may process the data and evaluate parameters, such as visual clarity, comfort, and the user's ability to seamlessly switch focus between different visual zones. Based on the analysis, the computer system 300 may fine-tune the lens parameters, ensuring an optimal balance between near and far vision correction. Results may be compiled into a report that provides insights into the user's visual performance and the recommended bifocal or multifocal lens prescription. As such, the computer system 300 may offer a dynamic, engaging, and precise approach to prescribing and adjusting multifocal lenses, representing a significant advancement over traditional methods that rely on static tests and subjective feedback.

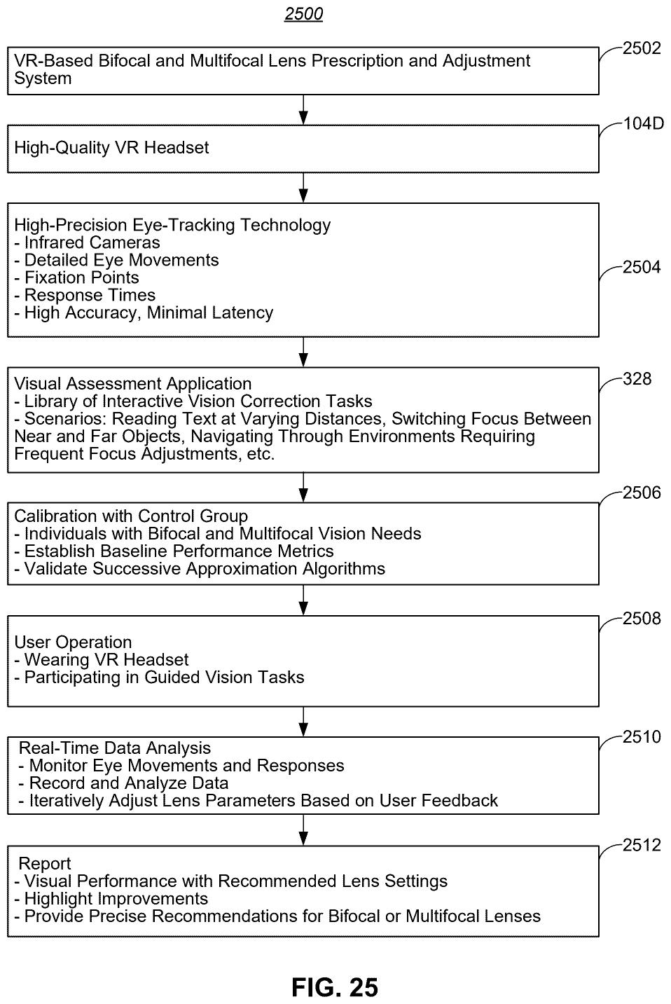

FIG. 25 is a flow diagram of an example vision test process 2500 for prescribing and adjusting multifocal lenses in a 3D virtual environment, in accordance with some embodiments. The VR-based computer system 300 may be configured to enable a VR-based bifocal and multifocal lens prescription and adjustment system 2502. The computer system 300 may include a VR headset 104D that includes an eye-tracking camera 366 (FIG. 3). The eye-tracking technology may include an infrared camera (e.g., camera 366) configured to capture (operation 2504) eye movements, fixation points, and response times with high accuracy and minimal latency. In some embodiments, when a visual assessment application 328 is executed, a library of interactive cognitive tasks may be applied to test various aspects of vision correction with bifocal and multifocal lenses. These tasks include scenarios where the user may be prompted to read text at varying distances, switch focus between near and far objects, and navigate through environments that require frequent adjustments in focus.

In some embodiments, when hardware components and software modules may be integrated to form the VR-based bifocal and multifocal lens prescription and adjustment system 2502, the VR-based computer system 300 may be calibrated (operation 2506) using a control group of individuals with known bifocal and multifocal vision needs to establish baseline performance metrics and validate the accuracy of the adjustment algorithms. Users can operate (operation 2508) the calibrated computer system 300 by wearing the VR headset and participating in the guided vision tasks within the virtual environments. The eye-tracking camera 366 may monitor their eye movements and responses to the lens simulations. The computer system 300 may iteratively adjust the bifocal and multifocal lens parameters based on user feedback, refining the settings to determine the optimal prescription. Image or video data recorded by the camera 366 may be analyzed (operation 2510) in real time by the software modules (e.g., visual assessment application 328, data processing module 330 in FIG. 3). In some implementations, the user may receive a report 2512 outlining their visual performance with the recommended lens settings, highlighting improvements and providing precise recommendations for bifocal or multifocal lenses. By these means, the computer system 300 may offer a precise, non-invasive, and user-friendly method for prescribing and adjusting bifocal and multifocal lenses, providing substantial benefits for both clinical applications and personal eye care routines.

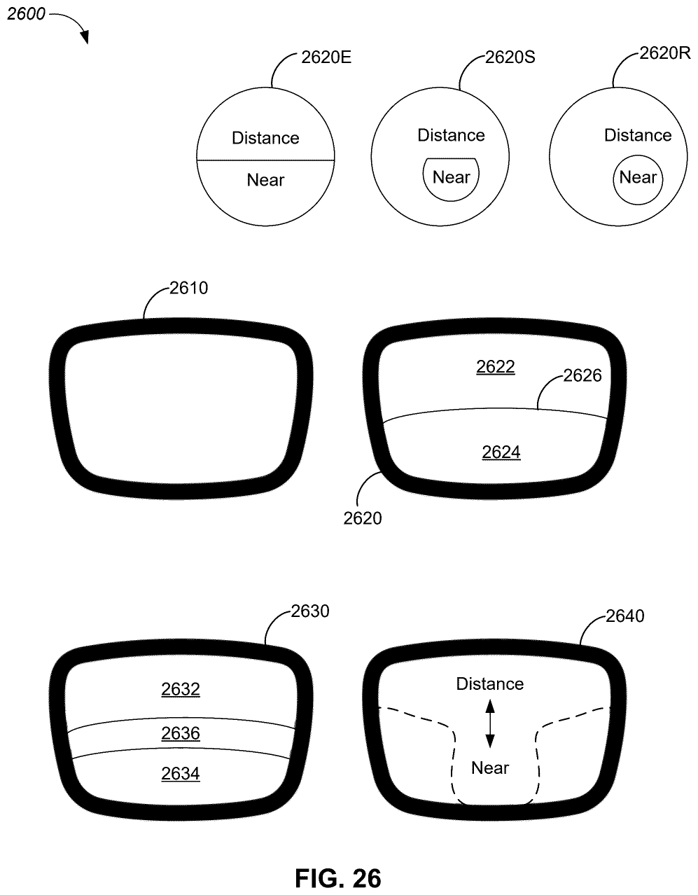

FIG. 26 is a set of example lenses 2600 including one or more focal lengths, in accordance with some embodiments. The set of lenses 2600 include a single vision lens 2610, a bifocal lens 2620, a trifocal lens 2630, and a progressive lens 2640. A single vision lens 2610 may have a single focal length and correct vision at a distance, whether it be near, intermediate, or far. The single vision lens 2610 may be prescribed for individuals with myopia (nearsightedness) or hyperopia (farsightedness) who need vision correction only for one range of distance. In contrast, the bifocal lens 2620, the trifocal lens 2630, and the progressive lens 2640 are collectively called multifocal lenses having more than one focal length.

The bifocal lens 2620 may have two distinct optical powers within the same lens: a first segment 2622 for distance vision and a separate second segment 2624 for near vision. The first segment 2622 and the second segment 2624 may be marked by a visible line 2626 separating the two segments 2622 and 2624. The bifocal lens 2620 may be used by individuals with presbyopia, which affects a user's ability to focus on close objects as they age. In some embodiments, each of the first segment 2622 and the second segment 2624 occupies a respective half of an executive bifocal lens 2620E. In some embodiments, the second segment 2624 is smaller than, and fully enclosed by, a lower portion of the first segment 2622 in a straight top bifocal lens 2620S or a round bifocal lens 2620R. The second segment 2624 has a flat top edge in the straight top bifocal lens 2620S and a round shape in the round bifocal lens 2620R.

The trifocal lens 2630 adds an intermediate vision correction segment 2636 between a distance segment 2632 and a near vision segment 2634, providing a more comprehensive range of vision correction. The trifocal lens 2630 may be beneficial for those who require sharp vision at multiple distances. The progressive lens 2640 (also called a no-line multifocal lens) may offer a seamless gradient of varying lens powers for distance, intermediate, and near vision correction. The progressive lens 2640 may allow for a smooth transition between different focal lengths, eliminating the visible lines found in bifocal and trifocal lenses, and providing a more natural visual experience for a wearer.

In some implementations, a multifocal lens may include a plurality of segments and have more than one focal length. The multifocal lens may be distinct from the bifocal lens 2620, the trifocal lens 2630, and the progressive lens 2640.

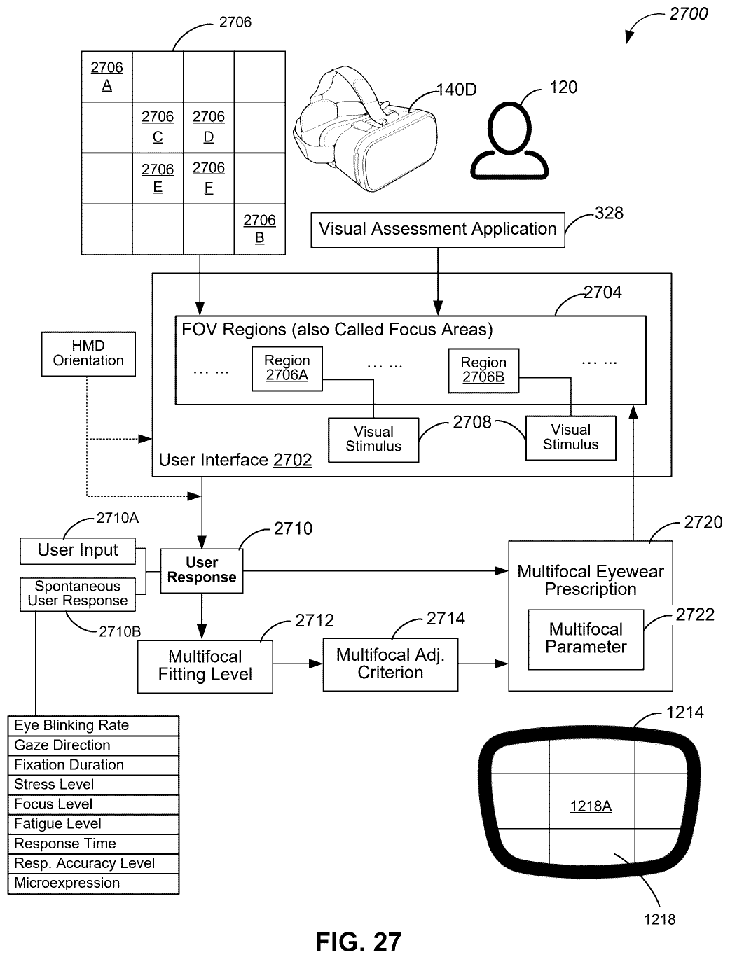

FIG. 27 is a flow diagram of an example vision test process 2700 for determining a multifocal eyewear prescription 2720, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 2702 corresponding to a 3D virtual environment. The computer device 140 may determine a multifocal eyewear prescription 2720 of a user 120 associated with the computer device 140. The multifocal eyewear prescription 2720 may include a multifocal parameter 2722 for a lens 1214 having a plurality of focal lengths. The computer device 140 may partition a field of view 2704 displayed on the user interface 2702 into a plurality of regions 2706 and display a visual stimulus 2708 successively in two distinct regions 2706A and 2706B of the user interface 2702. The computer device 140 may obtain user response data 2710 captured by one or more sensors 360 (FIG. 3) in response to the visual stimulus 2708 displayed in the two distinct regions 2706A and 2706B. Based on the user response data 2710, the multifocal parameter 2722 of the multifocal eyewear prescription 2720 may be adjusted.

In some embodiments, the plurality of regions 2706 of the field of view 2704 may include a grid of regions, and the two distinct regions 2706A and 2706B may not be immediately adjacent to each other.

In some embodiments, the lens 1214 may include a progressive lens 2640 (FIG. 26) having a gradient of varying lens powers for distance, intermediate, and near vision correction. The multifocal parameter 2722 may correspond to a gradient of lens powers. The computer device 140 may adjust the multifocal parameter 2722 further by modifying the gradient of varying lens powers in at least a portion of the lens. In some embodiments, the lens 1214 may include a bifocal lens 2620 (FIG. 26), and the multifocal parameter 2722 may correspond to three types of lenses 2620E, 2620S, and 2620R or a ratio between the first segment 2622 for distance vision and the separate second segment 2624 for near vision. The computer device 140 may select a different type of the three types of lenses 2620E, 2520S, and 2620R or adjust the ratio between the first segment 2622 for distance vision and the separate second segment 2624 for near vision. In some embodiments, the lens 1214 may include a trifocal lens 2630 (FIG. 26), and the multifocal parameter 2722 may correspond to correction powers of, or relative sizes among, an intermediate vision correction segment 2636, a distance segment 2632 and a near vision segment 2634. The computer device 140 may adjust the correction powers of, or the relative sizes among, the segments 2632-2636.

In some embodiments, based on the user response data 2710, the computer device 140 may determine a spontaneous user response 2710B having one or more response parameters of: an eye blinking rate, a gaze direction, a fixation duration, a stress level, a focus level, a response time, a response accuracy level, and a micro expression type. The multifocal parameter may be determined based on the one or more response parameters.

In some embodiments, the plurality of sensors 360 used to capture the user response data 2710 include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362.

In some embodiments, the computer device 140 may adjust the multifocal parameter 2722 by determining a multifocal fitting level 2712 based on the user response data 2710. The multifocal parameter 2722 may be adjusted in accordance with a determination that the multifocal fitting level 2712 satisfies a multifocal adjustment criterion 2714. For example, in some situations, the multifocal fitting level 2712 is substantially low, when the eye blinking rate determined based on the user response data 2710 is greater than a blinking rate threshold. The multifocal eyewear prescription 2720 is not a good fit for the user's eyes, and the multifocal adjustment criterion 2714 is satisfied, causing the computer device 140 to adjust the multifocal parameter 2722 of the multifocal eyewear prescription 2720 is not a good fit for the user's eyes.

In some embodiments, the computer device 140 may determine whether the HMD 312A is oriented forward when the visual stimulus 2708 are displayed in the two distinct regions 2706A and 2706B of the user interface 2702. The multifocal parameter 2722 may be adjusted based on the user response data 2710 in accordance with a determination that the HMD 312A is oriented forward.

Referring to FIG. 27, In some embodiments, the plurality of regions 2706 may include a grid of 3×3 regions, and the two distinct regions may be located on a top row and a bottom row of the grid, respectively. In some embodiments, the plurality of regions 2706 may include a grid of 3×3 regions, and the two distinct regions 2706A and 2706B may be diagonal to each other in the grid. The user response data 2710 may indicate how well the user's eyes respond to the visual stimulus 2708 switching between the two regions 2706A and 2706B, allowing the multifocal eyewear prescription 2720 to be adjusted accordingly. Existing eye vision tests do not display visual stimulus 2708 according to different dynamic display schemes, nor do they allow the multifocal eyewear prescription 2720 to be adjusted based on the visual stimulus 2708 switching between the two regions 2706A and 2706B in the field of view 2704.

In some embodiments, the lens 1214 may include a grid of lens portions 1218 each having a distinct focal length. Each lens portion 1218 may correspond to a subset of the multifocal eyewear prescription 2720 (e.g., one or more multifocal parameters 2722 of a set of one or more regions 2706). For example, a lens portion 1218A may correspond to regions 2706C, 2706D, 2706E, and 2706F.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 2702 corresponding to a 3D virtual environment. The computer device 140 may obtain a multifocal eyewear prescription 2720 of a user 120 associated with the computer device 140. The multifocal eyewear prescription 2720 may include a multifocal parameter 2722 for a lens 1214 having a plurality of focal lengths. Based on the multifocal parameter, the computer device may display a visual stimulus 2708 successively in a plurality of distinct regions 2706 of the 3D virtual environment. The computer device 140 may obtain a spontaneous user response 2710B in response to the visual stimulus 2708 displayed in the two distinct regions 2706A and 2706B. Based on the spontaneous user response 2710B, the computer device 140 automatically adjust the multifocal parameter 2722 of the multifocal eyewear prescription 2720.

In some embodiments, the visual stimulus 2708 is displayed in the plurality of distinct regions 2706 according to a predefined temporal order (e.g., an order 1310 in FIG. 13). In some embodiments, the visual stimulus 2708 may be displayed in the plurality of distinct regions 2706 randomly.

FIG. 28 is a flow diagram of an example process 2800 for determining a multifocal parameter 2722 based on user response data 2710, in accordance with some embodiments. The one or more sensors 360 that may capture the user response data 2710 include an eye-tracking camera 366, and the user response data 2710 may include a sequence of eye images 2802. The computer device 140 may determine at least one of a gaze point 2804, an eyeball position 2806, and a pupil size 2808 in each eye image 2802. Further, in some embodiments, the computer device 140 may determine an eye movement trace 2810 of the gaze point 2804, the eyeball position 2806, or the pupil size 2808 among the sequence of eye images 2802. The computer device 140 may further determine a multifocal fitting level 2812 based on the eye movement trace 2810, and the multifocal parameter 2722 may be adjusted in accordance with a determination that the multifocal fitting level 2712 satisfies a multifocal adjustment criterion 2714. For example, in some situations, the eye movement trace 2810 indicates that the multifocal fitting level 2712 is substantially low (e.g., the user's eyes have excessive movement), and the multifocal adjustment criterion 2714 is satisfied, causing the computer device 140 to adjust the multifocal parameter 2722.

Additionally, in some embodiments, the computer device 140 may determine an eye movement trace 2810 of the gaze point 2804, the eyeball position 2806, or the pupil size 2808 among the sequence of eye images 2802, extract a plurality of eye movement samples 2814 from the eye movement trace 2810, and apply a multifocal adjustment model 2816 to process a plurality of eye movement samples 2814 and adjust the multifocal parameter 2722.

### Machine-Learning-Based Contact Lens Fitting Analysis

Some implementations of this application include a VR-based computer system 300 configured to evaluate and recommend specialized contact lenses through immersive scenarios. The computer system 300 may include a VR headset 104D that includes an eye-tracking camera 366 (FIG. 3). The eye-tracking camera 366 may include an infrared camera configured to capture eye movements and fixation patterns with high accuracy and minimal latency. In some embodiments, a visual assessment application 328 may be executed to generate realistic, interactive virtual environments. A user 120 may wear the VR headset and perform a series of tasks that replicate everyday activities under various visual conditions, such as low light, bright light, and digital screen exposure. In some embodiments, the eye-tracking camera 366 may monitor the user's gaze direction, fixation stability, and visual response accuracy, while the visual assessment application 328 adjusts the visual scenarios to simulate the effects of different contact lens prescriptions. This approach may assess in real time how various specialized contact lenses enhance visual performance in diverse settings.

In some embodiments, the VR-based computer system 300 may incorporate a range of immersive scenarios configured to challenge different aspects of vision, such as reading fine print, recognizing faces, navigating through a busy environment, and working on a computer. A user application 324 (e.g., visual assessment application 328 in FIG. 3) may process the data and evaluate parameters, such as visual clarity, comfort, and user satisfaction with each simulated lens type. Based on the analysis, the system recommends specialized contact lenses customized based on the user's specific visual needs, such as lenses configured for astigmatism, multifocal lenses for presbyopia, or lenses with enhanced UV protection. Results may be compiled into a report that provides insights into the user's visual performance and the recommended contact lens prescription. As such, the computer system 300 may offer a dynamic, engaging, and precise approach to evaluating and prescribing specialized contact lenses, representing a significant advancement over traditional static vision tests.

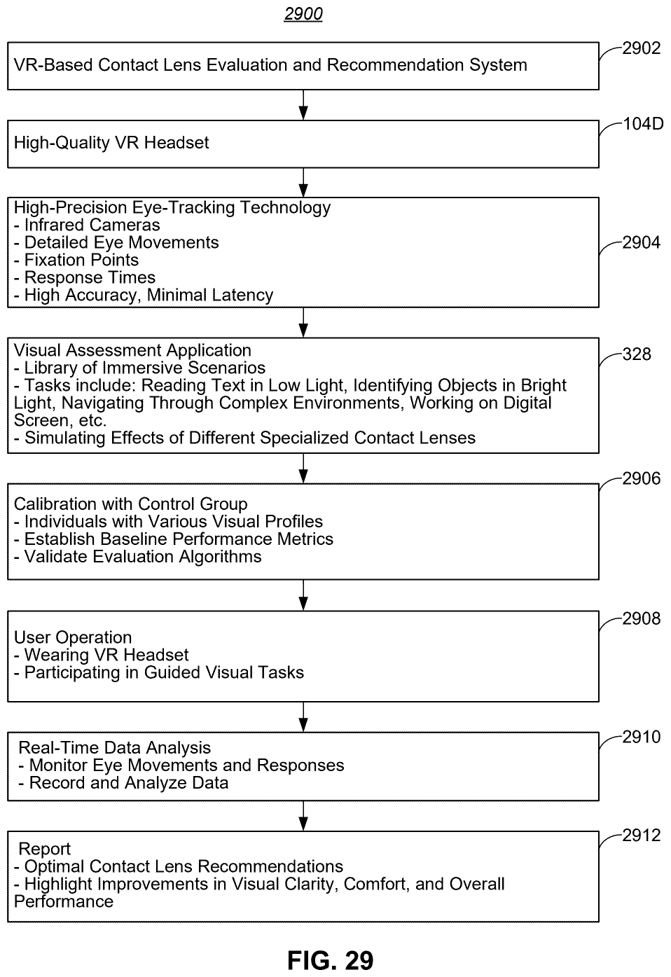

FIG. 29 is a flow diagram of an example vision test process 2900 for contact lens fitting in a 3D virtual environment, in accordance with some embodiments. The VR-based computer system 300 may be configured to enable a VR-based contact lens evaluation and recommendation system 2902. The computer system 300 may include a VR headset 104D that includes an eye-tracking camera 366 (FIG. 3). The eye-tracking technology may include an infrared camera (e.g., camera 366) configured to capture (operation 2904) eye movements, fixation points, and response times with high accuracy and minimal latency. In some embodiments, when a visual assessment application 328 is executed, a library of interactive cognitive tasks may be applied to test various aspects of visual performance under different conditions. These scenarios may include tasks where users may be prompted to read text in low light, identify objects in bright light, navigate through complex environments, and work on digital screens, simulating the effects of different specialized contact lenses.

In some embodiments, when hardware components and software modules may be integrated to form the VR-based contact lens evaluation and recommendation system 2902, the VR-based computer system 300 may be calibrated (operation 2906) using a control group of individuals with various visual profiles to establish baseline performance metrics and validate the accuracy of the evaluation algorithms. Users can operate (operation 2908) the calibrated computer system 300 by wearing the VR headset and participating in the guided visual tasks within the virtual environments. The eye-tracking camera 366 may monitor their eye movements and responses to the simulated contact lenses. Image or video data recorded by the camera 366 may be analyzed (operation 2910) in real time by the software modules (e.g., visual assessment application 328, data processing module 330 in FIG. 3). In some implementations, the user may receive a report 2912 outlining the optimal contact lens recommendations, highlighting improvements in visual clarity, comfort, and overall performance. By these means, the computer system 300 may offer a precise, non-invasive, and user-friendly method for evaluating and recommending specialized contact lenses, providing substantial benefits for both clinical applications and personal eye care routines.

FIG. 30 is a flow diagram of an example vision test process 3000 for checking contact lens fitting, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 3002 corresponding to a 3D virtual environment. The computer device 140 may display visual content 3004 continuously for a duration of time 3006 in the 3D virtual environment. The visual content 3004 may be displayed with predefined display parameters 3008 associated with contact lens fitting. The computer device 140 may obtain a stream of sensor data 342 measured by the one or more sensors 360 and apply at least a contact lens fitting model 3010 to generate a contact lens fitting profile 3012 for a user 120 associated with the computer device 104 based on the stream of sensor data 342. In an example, the user 120 may wear contact lenses for computer work, and the visual content 3004 may include a computer screen image rendered in an arm's length of the user 120.

In some embodiments, the contact lens fitting profile 3012 includes at least one of: a fitting level of contact lenses 3012A worn by the user 120, one or more potential eye conditions 3012B and one or more associated occurrence probabilities, a suggested prescription adjustment 3012C, and one or more recommendations of contact lens types 3012D. Further, in some embodiments, the one or more potential eye conditions 3010C include a subset of eye redness 3010-1, burning and itchiness 3010-2, eye discharge 3010-3, grittiness 3010-4, light sensitivity 3010-5, blurry vision 3010-6, and dry eye 3010-7.

In some embodiments, the computer device 140 may determine a plurality of sequential user responses 3014 to the visual content 3004 based on the stream of sensor data 342 and apply the contact lens fitting model 3010 to generate the contact lens fitting profile 3012. Further, in some embodiments, the plurality of sequential user responses 3014 may include one or more of: an eye blinking rate, a gaze direction, a fixation duration, a stress level, a focus level, an eye dryness level, an eye redness level, a fatigue level, a response time, a response accuracy level, and a micro expression type. In other words, the sequential user responses 3014 may include a spontaneous user response 3014B. The plurality of sensors 360 may include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. The sensor data 342 may be processed to determine the spontaneous user response 3014B automatically without receiving an active user input 3014A. In some embodiments, the user response 3014 includes a user input 3014A captured by a subset of one or more sensors of the computer device 104, and the one or more sensors include a forward facing camera 378 (FIG. 3) for detecting a hand gesture, a microphone 380 (FIG. 3) for collecting an audio response, or a controller 390 (FIG. 3) for receiving a user physical force.

In some embodiments, the predefined display parameters 3008 include a plurality of corner display parameters each of which is substantially close to a respective display parameter limit, and the contact lens fitting profile 3012 is generated under a stressed display condition. For example, the visual content 3004 may be displayed at an elevated brightness level, and the user's eyes may be tested to evaluate whether the contact lenses can still fit reasonable when exposed to the elevated brightness level, particularly for the time duration 3006.

In some embodiments, the contact lens fitting profile 3012 includes a light sensitivity level 3010-5. The computer device 140 may adjust a color scheme 3008A, a contrast level 3008B, or a brightness level 3008C of the HMD 312A.

In some embodiments, the stream of sensor data 342 are captured according to a temporal window 1510 (e.g., in FIGS. 15 and 21A). Each of the one or more sensors 360 may have a respective sampling rate and provide a subset of sensor data 342 based on the respective sampling rate. The temporal window 1510 may move along a time axis 1512. Further, in some embodiments, for each of the one or more sensors 360, the computer device 240 may apply a sensor feature extraction model to process the subset of sensor data and generate a respective sensor feature vector. A response monitoring model may be applied to process respective sensor feature vectors of the one or more sensors 360 and generate a respective sequential user response 3014 corresponding to the temporal window 1510. More details on tracking sequential user response 3014 are also explained above with reference to the sequential response 2022 applied in the process 2000 in FIG. 21A.

In some embodiments, the contact lens fitting profile 3012 is tracked during an extended duration of time (e.g., within 6 months) to monitor an eye health condition associated with contact lens wearing. Particularly, when the user 120 can implement the vision test process 3000 at home using the computer device 140 without visiting an optician's office, the computer device 140 may provide the contact lens fitting profile 3012 (e.g., eye redness 3010-1) in real time, and track development of the contact lens fitting profile 3012 over the extended duration of time.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. The computer device 140 may obtain a plurality of eye images 3020 captured by an eye-tracking camera 366 (e.g., FIGS. 3 and 28), and generate a current contact lens fitting profile 3012C for a user 120 associated with the computer device 140 based on the plurality of eye images 3020.

In some embodiments, the computer device 140 may extract a historical contact lens fitting profile 3012H and compare the historic contact lens fitting profile 3012H and the current contact lens fitting profile 3012C to identify a profile change 3016.

In some embodiments, visual content 3004 may be continuously displayed for a duration of time 3006 in the 3D virtual environment. The visual content 3004 may be displayed with predefined display parameters 3008 associated with contact lens fitting, and the plurality of eye images 3020 may be captured while the visual content 3004 is displayed.

In some embodiments, the computer device 140 may determine a spontaneous user response 3014B based on the plurality of eye images 3020. The spontaneous user response 3014B may include one or more of: an eye blinking rate, a gaze direction, a fixation duration, a stress level, a focus level, a response time, a response accuracy level, and a micro expression type.

### Content Rendering for Multifocal Eyewear Fitting

Some implementations of this application include a VR-based computer system 300 configured to measure how well users can adapt to different visual correction techniques, such as progressive lenses. The computer system 300 may include a VR headset 104D that includes an eye-tracking camera 366 (FIG. 3). The eye-tracking camera 366 may include an infrared camera configured to capture eye movements and fixation patterns with high accuracy and minimal latency. In some embodiments, a visual assessment application 328 may be executed to generate dynamic visual environments that simulate the use of various visual correction methods. A user 120 may wear the VR headset and perform a series of tasks that replicate real-world activities, such as reading, driving, and navigating through different environments. In some embodiments, the eye-tracking camera 366 may monitor the user's gaze direction, fixation stability, and visual response accuracy, while the visual assessment application 328 adjusts the visual simulations to reflect the effects of different correction techniques, such as bifocals, trifocals, and progressive lenses.

In some embodiments, the VR-based computer system 300 may incorporate a range of scenarios configured to test and measure the user's adaptation to these visual correction techniques. Example tasks include, but are not limited to, transitioning focus between near and far objects, navigating complex visual scenes, and performing activities that require peripheral vision. A user application 324 (e.g., visual assessment application 328 in FIG. 3) may process the data and evaluate parameters, such as visual clarity, comfort, and the speed of adaptation to each correction method. Results may be compiled into a report that provides insights into the user's adaptation performance, highlighting any challenges or areas of difficulty. Based on this analysis, the computer system 300 may recommend visual correction technique for the user, ensuring optimal visual performance and comfort. As such, the computer system 300 may offer a dynamic, engaging, and precise approach to evaluating visual correction techniques, representing a significant advancement over traditional static vision tests.

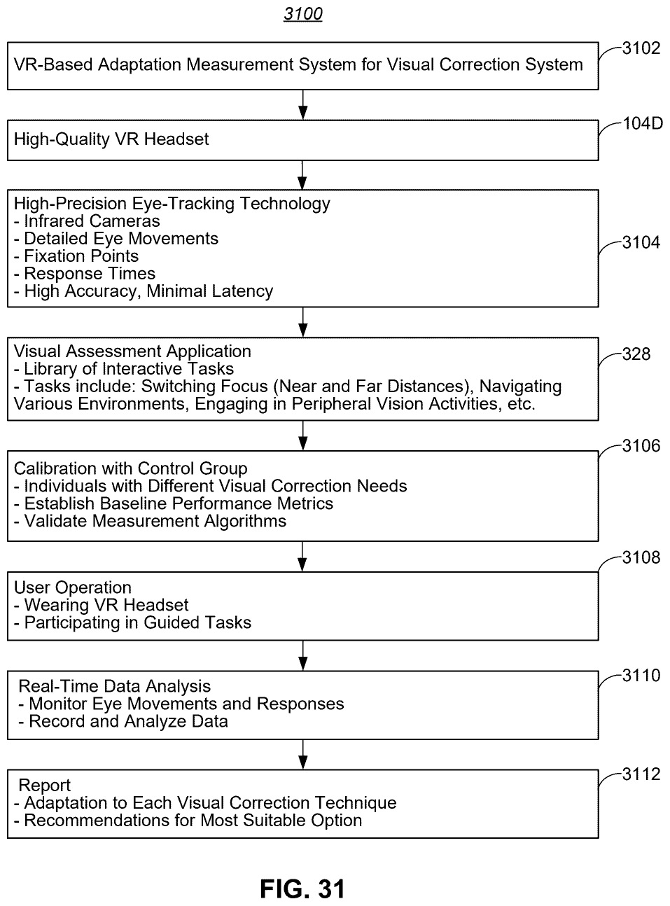

FIG. 31 is a flow diagram of an example process 3100 for rendering content for multifocal eyewear fitting in a 3D virtual environment, in accordance with some embodiments. The VR-based computer system 300 may be configured to enable a VR-based adaptation measurement system 3102. The computer system 300 may include a VR headset 104D that includes an eye-tracking camera 366 (FIG. 3). The eye-tracking technology may include an infrared camera (e.g., camera 366) configured to capture (operation 3104) eye movements, fixation points, and response times with high accuracy and minimal latency. In some embodiments, when a visual assessment application 328 is executed, a library of interactive cognitive tasks may be applied to test user adaptation to different visual correction methods. These tasks may include scenarios where users may be prompted to switch focus between near and far distances, navigate through various environments, and engage in activities that require extensive use of peripheral vision.

In some embodiments, when hardware components and software modules may be integrated to form the VR-based adaptation measurement system 3102, the VR-based computer system 300 may be calibrated (operation 3106) using a control group of individuals with different visual correction needs to establish baseline performance metrics and validate the accuracy of the adaptation measurement algorithms. Users can operate (operation 3108) the calibrated computer system 300 by wearing the VR headset and participating in the guided tasks within the virtual environments. The eye-tracking camera 366 may monitor their eye movements and responses to the simulated visual correction techniques. Image or video data recorded by the camera 366 may be analyzed (operation 3110) in real time by the software modules (e.g., visual assessment application 328, data processing module 330 in FIG. 3). In some implementations, the user may receive a report 3112 outlining their adaptation to each visual correction technique, providing recommendations for the most suitable option. By these means, the computer system 300 may offer a precise, non-invasive, and user-friendly method for measuring and enhancing user adaptation to various visual correction methods, providing substantial benefits for both clinical applications and personal eye care routines.

FIG. 32 is a flow diagram of an example vision test process 3200 for preparing an eyewear, in accordance with some embodiments. A vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface corresponding to a 3D virtual environment. The computer device 140 may obtain a comprehensive prescription 3220 (e.g. lens prescription 1220) for an eyewear 1214 having a plurality of lens portions 1218 (e.g., in FIG. 12), and each lens portion 1218 may correspond to one or more regions 1320 of a field of view 1300 (e.g., FIG. 13) and have a respective prescription parameter 3222 (e.g. corrective measures 1228). In some embodiments, a lens 1214 of the eyewear may be evenly divided to provide the plurality of lens portions 1218.

The computer device 140 may generate a bifocal filter 3202, a trifocal filter 3204, and a progressive filter 3206 based on the comprehensive prescription 3220. The computer device 140 may obtain 3D visual content 3208 for display on the user interface, and render three versions 3212, 3214, and 3216 of the 3D visual content 3004 based on the bifocal filter 3202, the trifocal filter 3204, and the progressive filter 3206, respectively. In some embodiments, the computer device 140 may obtain a user selection 3218 of one of the three version 3212, 3214, and 3216 of the 3D visual content. Based on the user selection 3218, the computer device 140 may simplify the comprehensive prescription 3220 to a multifocal prescription 3224 (e.g., to one of the bifocal lens 2620, the trifocal lens 2630, and the progressive lens 2640, which may have a scheme of combining different focal lengths on the same lens 1214). Further, in some embodiments, based on the multifocal prescription 3224, the computer device 140 may generate a set of one or more instructions 3226 to be sent to an eyewear manufacturing machine 3228 to make a lens (e.g., lens 1214 in FIG. 12) based on the multifocal prescription 3224.

In some embodiments, the computer device 140 may partition a field of view (e.g., field of view 1300) displayed on the user interface into a plurality of regions (e.g., regions 1320). The prescription 3220 of the eyewear 1214 may correspond to a filter map 1210 associating the plurality of regions 1320 with respective vision correction filters 1208 (e.g., in FIGS. 12 and 13), and each region 1320 may be associated with one or more filter settings 1212 of the respective vision correction filter 1208. Stated another way, the computer device 140 may apply the respective vision correction filters 1208 to different regions 1320 of the field of view 1300 to mimic different lens portions 1218 of the eyewear lens 1214 for improving perception of the visual content 3208.

In some embodiments, the computer device 140 may identify a selection of an eyewear lens 1214 (e.g., having a particular shape). Based on the selection of the eyewear lens 1214, the computer device 140 may adjust the bifocal filter 3202, the trifocal filter 3204, and the progressive filter 3206. When the particular shape of the eyewear lens 1214 is selected, the three versions 3212, 3214, and 3216 of the 3D visual content 3004 may be adjusted, e.g., based on the particular shape. In some embodiments, the user 120 may be prompted to

In some embodiments, the computer device 140 may partition a field of view 1300 displayed on the user interface into a plurality of regions 1320 substantially evenly.

In some embodiments, for each of the three versions 3212, 3214, and 3216 of the 3D visual content 3208, the computer device 140 may obtain a plurality of sensor signals from a plurality of sensors 360 (FIG. 3), and determine a stress level 1224 based on the plurality of sensor signals in response to the respective version 3212, 3214, or 3216 of the 3D visual content 3208.

In some embodiments, the user 120 may be prompted to provide a user response 3210, which may include a user input 3210A captured by a subset of one or more first sensors of the computer device 140. The one or more first sensors may include a forward-facing camera 378 (FIG. 3) for detecting a hand gesture, a microphone 380 (FIG. 3) for collecting an audio response, or a controller 390 (FIG. 3) for receiving a user physical force.

In some embodiments, the user response 3210 may include a spontaneous user response 3210B monitored by a subset of one or more second sensors of the computer device 140, e.g., automatically and without user intervention, and the one or more second sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362.

In some embodiments, for each of the three versions 3212, 3214, and 3216 of the 3D visual content 3208, the computer device 140 may obtain a plurality of sensor signals from a plurality of sensors 360 (FIG. 3) and determine a respective response parameter 3230 based on the plurality of sensor signals. The respective response parameter 3230 may include one or more of: an eye blinking rate, a gaze direction, a fixation duration, a stress level 1224, a focus level, a response time, a response accuracy level, and a micro expression type.

In some embodiments, the computer device 140 may provide respective response parameters 3230 of the three versions 3212, 3214, and 3216 of the 3D visual content 3208 to a generative artificial intelligence (AI) model 3232 and generate a message 3234 summarizing the respective response parameters 3230 of the three versions 3212, 3214, and 3216 of the 3D visual content 3208 using the generative AI model 3232.

Some implementations of this application are directed to implementing a vision test to get an eyewear prescription 1220. The eyewear prescription 1220 corresponds to the plurality of regions 1320 of the field of view 1300 shown in FIG. 13. The vision test may be implemented by a computer device 140 (e.g., a headset device 140D) that may further include one or more processors, memory storing instructions to be executed by the one or more processors, and an HMD 312A (FIG. 3). The computer device 140 may execute a user application 324 (e.g., a visual assessment application 328 in FIG. 3) configured to enable a virtual vision test and generate a VR user interface 1202 corresponding to a 3D virtual environment. The computer device 140 may obtain a comprehensive prescription 3220 for an eyewear having a plurality of lens portions 1218, and each lens portion 1218 may correspond to one or more respective region 1320 of a field of view 1300 and have a respective prescription parameter 3222. The computer device 140 may obtain 3D visual content 3208 for display on the user interface, generate a multifocal prescription 3224. The computer device 140 may iteratively render the 3D visual content 3208 based on the comprehensive prescription 3220 and simplify the comprehensive prescription 3220, until an eyewear fitting condition 3240 is satisfied.

In some embodiments, the computer device 140 may successively implement at least one set of operations of adjusting a bifocal filter 3202 and rendering the 3D visual content 3208 based on the bifocal filter 3202, adjusting a trifocal filter 3204 and rendering the 3D visual content 3208 based on the trifocal filter 3204, and adjusting a progressive filter 3206 and rendering the 3D visual content 3208 based on the progressive filter 3206.

### Illustration of the Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for implementing a vision test, comprising: at an electronic device including one or more processors, memory storing instructions, and a head-mounted display (HMD): executing a visual assessment application, including displaying a user interface to create a 3D virtual environment; partitioning a field of view displayed on the user interface into a plurality of regions; for each of the plurality of regions in the field of view, successively: rendering a respective visual pattern in the respective region; obtaining a user response to the respective visual pattern; adjusting a respective vision correction filter to the respective visual pattern based on the user response; combining respective vision correction filters corresponding to the plurality of regions to determine a prescription of an eyewear for a user associated with the electronic device.

Clause 2. The method of Clause 1, wherein the prescription of the eyewear includes a filter map associating the plurality of regions with the respective vision correction filters, each region associated with one or more filter settings of the respective vision correction filter.

Clause 3. The method of Clause 2, further comprising: identifying a selection of an eyewear lens; based on the selection of the eyewear lens, converting the filter map to a lens map, the lens map associating a plurality of lens portions of the eyewear lens with a plurality of correction powers.

Clause 4. The method of Clause 3, converting the filter map to the lens map further comprising, for each of the plurality of regions: identifying a respective lens portion of the eyewear lens; determining a respective correction power for the respective lens portion based on the respective filter settings of the respective vision correction filter corresponding to the respective region.

Clause 5. The method of Clause 3 or 4, wherein the eyewear lens is not evenly divided to provide the plurality of lens portions.

Clause 6. The method of any of Clauses 1-5, wherein the plurality of regions includes a first number of regions, and the first number is greater than 9.

Clause 7. The method of any of Clauses 1-6, wherein the field of view is divided substantially evenly to form the plurality of regions.

Clause 8. The method of any of Clauses 1-7, wherein the plurality of regions corresponds to a plurality of visual patterns jointly form an image frame, and the plurality of visual patterns is rendered on the user interface concurrently, and wherein the respective vision correction filters corresponding to the plurality of regions is adjusted jointly to determine the prescription of the eyewear.

Clause 9. The method of any of Clauses 1-8, wherein respective visual patterns of the plurality of regions is rendered successively to determine a respective subset of the prescription for each respective region.

Clause 10. The method of Clause 9, wherein the respective visual patterns are rendered successively in the plurality of regions according to a random order.

Clause 11. The method of any of Clauses 1-10, further comprising, for each of the plurality of regions: determining a stress level based on the user response to the respective visual pattern displayed in the respective region; and in accordance with a determination that the stress level satisfies a response criterion, associating the respective region with the respective vision correction filter.

Clause 12. The method of any of Clauses 1-11, wherein the user response includes a user input captured by a subset of one or more first sensors of the electronic device, and the one or more first sensors includes a forward facing camera for detecting a hand gesture, a microphone for collecting an audio response, and a controller for receiving a user physical force.

Clause 13. The method of any of Clauses 1-12, wherein the user response includes a spontaneous user response monitored by a subset of one or more second sensors of the electronic device, and the one or more second sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 14. The method of any of Clauses 1-13, further comprising: determining whether the HMD is oriented forward, wherein the visual pattern is rendered, and the user response is obtained and processed in accordance with a determination that the HMD is oriented forward.

Clause 15. A method for implementing a vision test, comprising: at an electronic device including one or more processors, memory storing instructions, and a head-mounted display (HMD): executing a visual assessment application, including displaying a user interface to create a 3D virtual environment; partitioning a field of view displayed on the user interface into a plurality of regions; determining one or more respective corrective measures for each of the plurality of regions in the field of view; and determine a prescription of an eyewear for a user associated with the electronic device, wherein the prescription of the eyewear includes a map associating each of the plurality of regions with one or more respective corrective measures.

Clause 16. The method of Clause 15, further comprising, for each of the plurality of regions in the field of view, successively: rendering a respective visual pattern in the respective region; obtaining a user response to the respective visual pattern; adjusting a respective vision correction filter to the respective visual pattern based on the user response; and generating the one or more respective corrective measures based on one or more filter settings of the respective vision correction filter.

Clause 17. A method for implementing a vision test, comprising: at an electronic device including one or more processors, memory storing instructions, and a head-mounted display (HMD): executing a visual assessment application, including displaying a user interface to create a 3D virtual environment corresponding to a field of view of a user associated with the electronic device; rendering a visual pattern in the field of view; applying a vision correction filter to the visual pattern; obtaining a set of user response data captured by a plurality of sensors in response to the visual pattern; determining whether the set of user response data satisfy a response quality criterion; and dynamically adjusting filter parameters of the vision correction filter based on the set of user response data, until the set of user response data satisfy the response quality criterion.

Clause 18. The method of Clause 17, wherein the visual pattern is rendered in one of a plurality of regions of the field of view, and the vision correction filter is configured to mimic a lens portion of an eyewear lens for improving perception of the visual pattern in the one of the plurality of regions of the field of view.

Clause 19. The method of Clause 18, further comprising: identifying a selection of the eyewear lens; and based on the selection of the eyewear lens, identifying the lens portion on the eyewear lens.

Clause 20. The method of Clause 18 or 19, further comprising: in accordance with a determination that the set of user response data satisfy the response quality criterion, converting the filter parameters of the vision correction filter to a set of one or more eye prescription parameters associated with the lens portion of the eyewear lens for the user.

Clause 21. The method of Clause 20, wherein the set of one or more eye prescription parameters associated with the lens portion of the eyewear lens includes one or more of: Sphere, Cylinder, Axis, ADD, PD, Prism, and Base.

Clause 22. The method of any of Clauses 17-21, further comprising: determining whether the HMD is oriented forward, wherein the visual pattern is rendered, and the set of user response data is processed in accordance with a determination that the HMD is oriented forward.

Clause 23. The method of any of Clauses 17-22, determining whether the set of user response data satisfy the response quality criterion further comprising: determining a plurality of response parameters based on the set of response data; determining a combination of the plurality of response parameters; and determining whether the combination of the plurality of response parameters satisfies the response quality criterion.

Clause 24. The method of any of Clauses 17-23, wherein the set of response data is captured in a temporal window, and each of the plurality of sensors has a respective sampling rate and provides a subset of response data items based on the respective sampling rate, and wherein the temporal window moves along a time axis.

Clause 25. The method of Clause 24, wherein the plurality of response parameters is determined based on the temporal window, and includes one or more of: an eye blinking rate, a gaze direction, a fixation duration, a stress level, a focus level, a response time, a response accuracy level, and a micro expression type.

Clause 26. The method of any of Clauses 17-25, further comprising: for each of the plurality of sensors, applying a sensor feature extraction model to process a subset of response data and generate a respective sensor feature vector; and applying a response monitoring model to process respective sensor feature vectors of the plurality of sensors and generate a response quality indicator indicating whether the set of user response data satisfy the response quality criterion.

Clause 27. The method of Clause 26, wherein the response monitoring model processes a visual pattern identification, display parameters of the visual pattern, and a location of the visual pattern jointly with the respective sensor feature vectors.

Clause 28. The method of Clause 27, further comprising, at a server: collecting training data from a plurality of eye patients, the training data including response data of the plurality of eye patients associated with the visual pattern; and training the response monitoring model based on the training data.

Clause 29. The method of any of Clauses 17-28, wherein the plurality of sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 30. A method for implementing a vision test, comprising: at an electronic device including one or more processors, memory storing instructions, and a head-mounted display (HMD): rendering a visual pattern in the field of view; applying a vision correction filter to the visual pattern; obtaining a set of user response data captured by a plurality of sensors in response to the visual pattern; adjusting filter parameters of the vision correction filter based on the set of user response data; in accordance with a determination that the set of user response data satisfy a response quality criterion, generating a prescription of an eyewear based on the filter parameters of the vision correction filter.

Clause 31. The method of Clause 30, wherein the visual pattern is rendered in at least one of a plurality of regions of the field of view, and the vision correction filter is configured to mimic a lens portion of an eyewear lens for improving perception of the visual pattern in the one of the plurality of regions of the field of view.

Clause 32. The method of Clause 30 or 31, wherein the visual pattern covers a plurality of regions of the field of view, and each region corresponds to a subset of respective filter parameters, adjusting the filter parameters of the vision correction filter further comprising, during each of a plurality of iteration: adjusting the subset of respective filter parameters for at least one region based on the set of user response data.

Clause 33. A method of implementing a vision test, comprising: at an electronic device having a head-mounted display (HMD), one or more sensors, one or more processors, and memory: executing a visual assessment application, including displaying a user interface to create a 3D virtual environment; displaying visual content continuously for an extended duration of time in the 3D virtual environment, wherein the visual content is displayed with predefined display parameters associated with a screen usage; obtaining a stream of sensor data measured by the one or more sensors; determining a plurality of sequential user responses to the visual content based on the stream of sensor data; and applying at least a screen usage prediction model to generate a screen usage guidance profile for the user based on the plurality of sequential user responses.

Clause 34. The method of Clause 33, wherein the screen usage guidance profile includes one or more: a screen size, a color scheme, a font size, a screen angle, a screen height, a background lighting condition, and a screen use time limit.

Clause 35. The method of Clause 33 or 34, wherein the screen usage guidance profile includes a brightness level and a contrast level of a screen to be used by the user.

Clause 36. The method of any of Clauses 33-35, wherein the screen usage guidance profile requires that the brightness level of the screen varies with a time spent on the screen, and the method further comprises automatically adjusting the brightness level of the screen.

Clause 37. The method of any of Clauses 33-36, wherein the screen usage guidance profile requires that the contrast level of the screen varies with a time spent on the screen, and the method further comprises automatically adjusting the contrast level of the screen.

Clause 38. The method of any of Clauses 33-37, wherein the screen usage guidance profile includes a color scheme, and the method further comprises, automatically: applying a first color scheme to the user interface; and in accordance with a determination that a time spent on a screen by the user is greater than a screen time limit, applying a second color scheme to the user interface.

Clause 39. The method of any of Clauses 33-38, wherein the plurality of sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 40. The method of any of Clauses 33-39, wherein the plurality of sequential user responses includes one or more of: an eye blinking rate, a gaze direction, a fixation duration, a stress level, a focus level, a fatigue level, a response time, a response accuracy level, and a micro expression type.

Clause 41. The method of any of Clauses 33-40, wherein the stream of sensor data is captured according to a temporal window, and each of the one or more sensors has a respective sampling rate and provides a subset of sensor data based on the respective sampling rate, and wherein the temporal window moves along a time axis.

Clause 42. The method of Clause 41, further comprising: for each of the one or more sensors, applying a sensor feature extraction model to process the subset of sensor data and generate a respective sensor feature vector; and applying a response monitoring model to process respective sensor feature vectors of the one or more sensors and generate a respective sequential user response corresponding to the temporal window.

Clause 43. The method of any of Clauses 33-42, wherein the predefined display parameters includes a plurality of corner display parameters each of which is substantially close to a respective display parameter limit.

Clause 44. A method of implementing a vision test, comprising: at an electronic device having a head-mounted display (HMD), one or more sensors, one or more processors, and memory: displaying visual content continuously for an extended duration of time in a 3D virtual environment, wherein the visual content is displayed with predefined display parameters associated with a screen usage; obtaining a stream of sensor data; determining a plurality of sequential user responses to the visual content based on the stream of sensor data; and generating a screen usage guidance profile for the user based on the plurality of sequential user responses, the screen usage guidance profile including at least a time-dependent display parameter.

Clause 45. The method of Clause 44, wherein the time-dependent display parameter includes one or more of: a color scheme, a font size, a background lighting condition, a contrast level, and a brightness level.

Clause 46. The method of Clause 44, wherein the time-dependent display parameter includes least two settings of: a color scheme, a font size, a background lighting condition, a contrast level, and a brightness level, and the two settings have temporal dependences that are independent of one another.

Clause 47. A method of implementing a vision test, comprising: at an electronic device having a head-mounted display (HMD), one or more sensors, one or more processors, and memory: executing a visual assessment application, including displaying a user interface to create a 3D virtual environment; displaying a predefined video clip in the 3D virtual environment, the predefined video clip including a plurality of visual sessions corresponding to a sequence of vision tests; while the predefined video clip is played, obtaining a stream of sensor data measured by the one or more sensors; and determining a plurality of first response parameters to the sequence of vision tests based on the stream of sensor data.

Clause 48. The method of Clause 47, further comprising, for each of one or more subsequent iterations: repeating display of the predefined video clip in the 3D virtual environment; and determining a plurality of second response parameters.

Clause 49. The method of Clause 48, further comprising tracking a variation of response parameters based on the plurality of first response parameters and the plurality of second response parameters of each subsequent iteration.

Clause 50. The method of Clause 49, wherein each subsequent iteration is implemented on a distinct day, and the variation of response parameters indicates chronic development of the user's eyesight.

Clause 51. The method of Clause 50, further comprising applying a chronic development model to process the plurality of first response parameters and the plurality of second response parameters of each subsequent iteration jointly and generate a chronic condition output associated with the variation of response parameters.

Clause 52. The method of Clause 51, wherein the chronic condition output includes one or more of: an eyesight drop rate, whether each of a plurality of known eye conditions newly occurs, whether each of a plurality of existing eye conditions gets worse or better, and whether further professional consultation is needed.

Clause 53. The method of any of Clauses 47-52, wherein the predefined video clip is displayed while a user associated with the electronic device is wearing an eyewear having a first eyewear prescription, and the plurality of first response parameters correspond to the eyewear having the first eyewear prescription.

Clause 54. The method of Clause 53, further comprising, while the user is wearing an eyewear having a second eyewear prescription: repeating display of the predefined video clip in the 3D virtual environment; determining a plurality of second response parameters; and comparing the plurality of first response parameters and the plurality of second response parameters; and based on a comparison result, determining whether the second eyewear prescription improves eyesight correction compared with the first eyewear prescription.

Clause 55. The method of any of Clauses 47-54, wherein the plurality of sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 56. The method of any of Clauses 47-55, wherein the plurality of first response parameters includes one or more of: an eye blinking rate, a gaze direction, a fixation duration, a stress level, a focus level, a fatigue level, a response time, a response accuracy level, and micro expression information.

Clause 57. The method of any of Clauses 47-56, wherein the stream of sensor data is captured according to a temporal window, and each of the one or more sensors has a respective sampling rate and provides a subset of sensor data based on the respective sampling rate, and wherein the temporal window moves along a time axis.

Clause 58. The method of Clause 57, further comprising: for each of the one or more sensors, applying a sensor feature extraction model to process the subset of response data and generate a respective sensor feature vector; applying a response monitoring model to process respective sensor feature vectors of the one or more sensors and generate a respective sequential user response corresponding to the temporal window; and combining respective sequential user responses of a set of successive temporal windows to determine the plurality of first response parameters.

Clause 59. A method of implementing a vision test, comprising: at an electronic device having a head-mounted display (HMD), one or more sensors, one or more processors, and memory: displaying a predefined video clip in a 3D virtual environment, the predefined video clip including a plurality of visual sessions corresponding to a sequence of vision tests; while the predefined video clip is played, obtaining a stream of sensor data measured by the one or more sensors; determining a current response feature vector indicating a user response to the sequence of vision tests based on the stream of sensor data; and determining a chronic vision change of a user associated with the electronic device based on a plurality of response feature vectors including a current response feature vector.

Clause 60. The method of Clause 59, wherein the plurality of response feature vectors further includes a set of one or more historical response feature vectors, the method further comprising: extracting the set of one or more historical response feature vectors; and applying a vision change model to process the plurality of response feature vectors to determine the chronic vision change.

Clause 61. A method for implementing a vision test, comprising: at an electronic device including one or more processors, memory storing instructions, and a head-mounted display (HMD): executing a visual assessment application, including displaying a user interface to create a 3D virtual environment; identifying a plurality of horizontal lines of sight; for each horizontal line of sight: rendering a respective visual stimulus on the respective horizontal line of sight; obtaining a user response to the respective visual stimulus; dynamically adjusting stimulus parameters of the respective visual stimulus based on the user response; based on the stimulus parameters associated with each horizontal line of sight, determine an eyewear prescription of an eyewear for a user associated with the electronic device, the eyewear prescription including prescription parameters corresponding to the plurality of horizontal lines of sight.

Clause 62. The method of Clause 61, wherein the stimulus parameters include at least a stimulus depth and a stimulus size, and the prescription parameters corresponding to each horizontal line of sight is determined based on the stimulus parameters of the respective visual stimulus associated with the respective horizontal line of sight.

Clause 63. The method of Clause 61 or 62, wherein every two immediately adjacent lines of sight of the plurality of horizontal lines of sight is separated by 15-30 degrees, inclusively.

Clause 64. The method of any of Clauses 61-63, wherein the eyewear prescription further includes prescription parameters corresponding to a plurality of lifted lines of sight or a plurality of lowered lines of sight.

Clause 65. The method of Clause 64, further comprising: for each of the plurality of lifted lines of sight or the plurality of lowered lines of sight, dynamically adjusting stimulus parameters of the respective visual stimulus based on a user response to a respective visual stimulus displayed on the respective line of sight.

Clause 66. The method of any of Clauses 61-65, wherein the eye prescription maps a plurality of lines of sight including the plurality of horizontal lines of sight with respective prescription parameters.

Clause 67. The method of Clause 66, further comprising: identifying a selection of an eyewear lens; based on the selection of the eyewear lens, converting the respective prescription parameters of the plurality of lines of sight to a lens map, the lens map associating a plurality of lens portions of the eyewear lens with a plurality of correction powers.

Clause 68. The method of Clause 67, converting the respective prescription parameters of the plurality of lines of sight to the lens map further comprising, for each of the plurality of lines of sight: identifying a respective lens portion of the eyewear lens; determining a respective correction power for the respective lens portion based on the respective prescription parameters corresponding to the respective line of sight.

Clause 69. The method of Clause 67 or 68, wherein the eyewear lens is not evenly divided to provide the plurality of lens portions.

Clause 70. The method of any of Clauses 61-69, wherein a horizontal field of view is divided substantially evenly to identify the plurality of horizontal lines of sight.

Clause 71. The method of any of Clauses 61-70, wherein the respective visual stimulus displayed on each horizontal line of sight includes a predefined visual stimulus.

Clause 72. The method of any of Clauses 61-71, wherein respective visual stimuli of the plurality of horizontal lines of sight is rendered successively to determine a respective subset of the eyewear prescription for each respective horizontal line of sight.

Clause 73. The method of Clause 72, wherein the respective visual patterns are rendered successively in the plurality of horizontal lines of sight according to a random order.

Clause 74. The method of any of Clauses 61-73, further comprising, for each of the plurality of horizontal lines of sight: determining a stress level based on the user response to the respective visual stimulus displayed in the respective horizontal line of sight; and in accordance with a determination that the stress level satisfies a response criterion, associating the respective horizontal line of sight with the respective stimulus parameters.

Clause 75. The method of any of Clauses 61-74, wherein the user response includes a user input captured by a subset of one or more sensors of the electronic device, and the one or more first sensors include a forward facing camera for detecting a hand gesture, a microphone for collecting an audio response, and a controller for receiving a user physical force.

Clause 76. The method of any of Clauses 61-75, wherein the user response includes a spontaneous user response monitored by a subset of one or more second sensors of the electronic device, and the one or more second sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 77. The method of any of Clauses 61-76, further comprising: determining whether the HMD is oriented forward, wherein the respective visual stimulus is rendered, and the user response is obtained and processed in accordance with a determination that the HMD is oriented forward.

Clause 78. The method of any of Clauses 61-77, wherein a first horizontal line of sight is immediately adjacent to a second horizontal line of sight, the method further comprising: setting initial parameters of the respective visual stimulus of the second horizontal line of sight based on at least the stimulus parameters determined for the respective visual stimulus of the first horizontal line of sight.

Clause 79. The method of any of Clauses 61-78, wherein the prescription parameters of each horizontal line of sight include one or more of: Sphere, Cylinder, Axis, ADD, PD, Prism, and Base.

Clause 80. A method for implementing a vision test, comprising: at an electronic device including one or more processors, memory storing instructions, and a head-mounted display (HMD): identifying a plurality of horizontal lines of sight; rendering a visual stimulus successively on the plurality of horizontal lines of sight; dynamically adjusting stimulus parameters of the visual stimulus based on a spontaneous user response; based on the stimulus parameters, determining an eyewear prescription of an eyewear for a user associated with the electronic device, the eyewear prescription including corrective measurements corresponding to the plurality of horizontal lines of sight.

Clause 81. The method of Clause 80, further comprising: obtaining a plurality of eye images captured by an eye-tracking camera; and determining the spontaneous user response based on the plurality of eye images.

Clause 82. The method of Clause 80 or 81, wherein the spontaneous user response includes one or more of: an eye blinking rate, a gaze direction, a fixation duration, a stress level, a focus level, a response time, a response accuracy level, and a micro expression type.

Clause 83. A method for implementing a vision test, comprising: at an electronic device comprising a head-mounted display (HMD), one or more processors and memory: determining a multifocal eyewear prescription of a user associated with the electronic device, wherein the multifocal eyewear prescription includes a multifocal parameter for a lens having a plurality of focal lengths; partition a field of view displayed on the user interface into a plurality of regions; displaying a visual stimulus successively in two distinct regions of the user interface; obtaining user response data captured by one or more sensors in response to the visual stimulus displayed in the two distinct regions; and based on the user response data, adjusting the multifocal parameter of the multifocal eyewear prescription.

Clause 84. The method of Clause 83, wherein the plurality of regions includes a grid of regions, and the two distinct regions are not immediately adjacent to each other.

Clause 85. The method of Clause 83 or 84, wherein the lens includes a progressive lens having a gradient of varying lens powers for distance, intermediate, and near vision correction, and adjusting the multifocal parameter further includes modifying the gradient of varying lens powers in at least a portion of the lens.

Clause 86. The method of any of Clauses 83-85, wherein the one or more sensors includes an eye-tracking camera, and the user response data includes a sequence of eye images captured, the method further comprising: determining at least one of a gaze point, an eyeball position, and a pupil size in each eye image.

Clause 87. The method of Clause 86, further comprising: determining an eye movement trace of the gaze point, the eyeball position, or the pupil size among the sequence of eye images; and determining a multifocal fitting level based on the eye movement trace, wherein the multifocal parameter is adjusted in accordance with a determination that the multifocal fitting level satisfies a multifocal adjustment criterion.

Clause 88. The method of Clause 86 or 87, further comprising: determining an eye movement trace of the gaze point, the eyeball position, or the pupil size among the sequence of eye images; extracts a plurality of eye movement samples from the eye movement trace; and applying a multifocal adjustment model to process a plurality of eye movement samples and adjust the multifocal parameter.

Clause 89. The method of any of Clauses 83-88, adjusting the multifocal parameter further comprising: based on the user response data, determining one or more response parameters of: an eye blinking rate, a gaze direction, a fixation duration, a stress level, a focus level, a response time, a response accuracy level, and a micro expression type, wherein the multifocal parameter is determined based on the one or more response parameters.

Clause 90. The method of any of Clauses 83-89, wherein the plurality of sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 91. The method of any of Clauses 83-90, adjusting the multifocal parameter further comprising: determining a multifocal fitting level based on the user response data, wherein the multifocal parameter is adjusted in accordance with a determination that the multifocal fitting level satisfies a multifocal adjustment criterion.

Clause 92. The method of any of Clauses 83-91, further comprising: determining whether the HMD is oriented forward when the visual stimulus is displayed in the two distinct regions of the user interface, wherein the multifocal parameter is adjusted based on the user response data in accordance with a determination that the HMD is oriented forward.

Clause 93. The method of any of Clauses 83-9, wherein the plurality of regions includes a grid of 3×3 regions, and the two distinct regions are diagonal to each other in the grid.

Clause 94. The method of any of Clauses 83-92, wherein the plurality of regions includes a grid of 3×3 regions, and the two distinct regions are located on a top row and a bottom row of the grid, respectively.

Clause 95. The method of any of Clauses 83-93, wherein the lens includes a grid of lens portions each having a distinct focal length.

Clause 96. A method of implementing a vision test, comprising: at an electronic device comprising a head-mounted display (HMD), one or more processors and memory: obtaining a multifocal eyewear prescription of a user associated with the electronic device, wherein the multifocal eyewear prescription includes a multifocal parameter for a lens having a plurality of focal lengths; based on the multifocal parameter, displaying a visual stimulus successively in a plurality of distinct regions of a 3D virtual environment; obtaining a spontaneous user response in response to the visual stimulus displayed in the two distinct regions; and based on the spontaneous user response, automatically, adjusting the multifocal parameter of the multifocal eyewear prescription.

Clause 97. The method of Clause 96, wherein the visual stimulus is displayed in the plurality of distinct regions according to a predefined temporal order.

Clause 98. The method of Clause 96 or 97, wherein the visual stimulus is displayed in the plurality of distinct regions randomly.

Clause 99. A method of implementing a vision test, comprising: at an electronic device having a head-mounted display (HMD), one or more sensors, one or more processors, and memory: executing a visual assessment application, including displaying a user interface to create a 3D virtual environment; displaying visual content continuously for an extended duration of time in the 3D virtual environment, wherein the visual content is displayed with predefined display parameters associated with contact lens fitting; obtaining a stream of sensor data measured by the one or more sensors; applying at least a contact lens fitting model to generate a contact lens fitting profile for a user associated with the electronic device based on the stream of sensor data.

Clause 100. The method of Clause 99, wherein the contact lens fitting profile includes at least one of: a fitting level of contact lenses worn by the user, one or more potential eye conditions and one or more associated occurrence probabilities, a suggested prescription adjustment, and one or more recommendations of contact lens types.

Clause 101. The method of Clause 100, wherein the one or more potential eye conditions includes a subset of eye redness, burning and itchiness, eye discharge, grittiness, light sensitivity, blurry vision, and dry eye.

Clause 102. The method of any of Clauses 99-101, further comprising: determining a plurality of sequential user responses to the visual content based on the stream of sensor data; and applying the contact lens fitting model to generate the contact lens fitting profile.

Clause 103. The method of Clause 102, wherein the plurality of sequential user responses includes one or more of: an eye blinking rate, a gaze direction, a fixation duration, a stress level, a focus level, an eye dryness level, an eye redness level, a fatigue level, a response time, a response accuracy level, and a micro expression type.

Clause 104. The method of any of Clauses 99-103, wherein the predefined display parameters include a plurality of corner display parameters each of which is substantially close to a respective display parameter limit, and the contact lens fitting profile is generated under a stressed display condition.

Clause 105. The method of any of Clauses 99-104, wherein the contact lens fitting profile includes a light sensitivity level, the method further comprising: adjusting a color scheme, a contrast level, or a brightness level of the HMD.

Clause 106. The method of any of Clauses 99-105, wherein the plurality of sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 107. The method of any of Clauses 99-106, wherein the stream of sensor data is captured according to a temporal window, and each of the one or more sensors has a respective sampling rate and provides a subset of sensor data based on the respective sampling rate, and wherein the temporal window moves along a time axis.

Clause 108. The method of Clause 107, further comprising: for each of the one or more sensors, applying a sensor feature extraction model to process the subset of sensor data and generate a respective sensor feature vector; and applying a response monitoring model to process respective sensor feature vectors of the one or more sensors and generate a respective sequential user response corresponding to the temporal window.

Clause 109. The method of any of Clauses 99-108, wherein the contact lens fitting profile is tracked during an extended duration of time to monitor an eye health condition associated with contact lens wearing.

Clause 110. A method of implementing a vision test, comprising: at an electronic device having a head-mounted display (HMD), one or more sensors, one or more processors, and memory: executing a visual assessment application, including displaying a user interface to create a 3D virtual environment; obtaining a plurality of eye images captured by an eye-tracking camera; and generating a current contact lens fitting profile for a user associated with the electronic device based on the plurality of eye images.

Clause 111. The method of Clause 110, further comprising: extracting a historical contact lens fitting profile; and comparing the historic contact lens fitting profile and the current contact lens fitting profile to identify a profile change.

Clause 112. The method of Clause 110 or 111, wherein the contact lens fitting profile includes at least one of: a fitting level of contact lenses worn by the user, one or more potential eye conditions and one or more associated occurrence probabilities, a suggested prescription adjustment, and one or more recommendations of contact lens types.

Clause 113. The method of any of Clauses 110-112, further comprising: displaying visual content continuously for an extended duration of time in the 3D virtual environment, wherein the visual content is displayed with predefined display parameters associated with contact lens fitting, and the plurality of eye images is captured while the visual content is displayed.

Clause 114. The method of any of Clauses 110-113, further comprising determining a spontaneous user response based on the plurality of eye images, wherein the spontaneous user response includes one or more of: an eye blinking rate, a gaze direction, a fixation duration, a stress level, a focus level, a response time, a response accuracy level, and a micro expression type.

Clause 115. A method for preparing an eyewear, comprising: at an electronic device including one or more processors, memory storing instructions, and a head-mounted display (HMD): executing a visual assessment application, including displaying a user interface to create a 3D virtual environment; obtaining a comprehensive prescription for an eyewear having a plurality of lens portions, each lens portion corresponding to a distinct region of a field of view and having a respective prescription parameter; generating a bifocal filter, a trifocal filter, and/or a progressive filter based on the comprehensive prescription; obtaining 3D visual content for display on the user interface; and rendering a plurality of versions of the first 3D visual content based on the bifocal filter, the trifocal filter, and/or the progressive filter.

Clause 116. The method of Clause 115, further comprising: obtaining a user selection of one of the three version of the 3D visual content; and based on the user selection, simplifying the comprehensive prescription to a multifocal prescription.

Clause 117. The method of Clause 116, further comprising, based on the multifocal prescription, generating a set of one or more instructions to be sent to an eyewear manufacturing machine to make a lens based on the multifocal prescription.

Clause 118. The method of any of Clauses 115-117, further comprising partitioning a field of view displayed on the user interface into a plurality of regions, wherein the prescription of the eyewear corresponds to a filter map associating the plurality of regions with respective vision correction filters, each region associated with one or more filter settings of the respective vision correction filter.

Clause 119. The method of any of Clauses 115-118, further comprising: identifying a selection of an eyewear lens; based on the selection of the eyewear lens, adjusting the bifocal filter, the trifocal filter, and the progressive filter.

Clause 120. The method of any of Clauses 115-119, wherein a lens of the eyewear is evenly divided to provide the plurality of lens portions.

Clause 121. The method of any of Clauses 115-120, further comprising partitioning a field of view displayed on the user interface into a plurality of regions substantially evenly.

Clause 122. The method of any of Clauses 115-121, further comprising, for each of the three versions of the 3D visual content: obtaining a plurality of sensor signals from a plurality of sensors; and determining a stress level based on the plurality of sensor signals in response to the respective version of the 3D visual content.

Clause 123. The method of any of Clauses 115-122, wherein the user response includes a user input captured by a subset of one or more first sensors of the electronic device, and the one or more first sensors includes a forward facing camera for detecting a hand gesture, a microphone for collecting an audio response, and a controller for receiving a user physical force.

Clause 124. The method of any of Clauses 115-123, wherein the user response includes a spontaneous user response monitored by a subset of one or more second sensors of the electronic device, and the one or more second sensors includes one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 125. The method of any of Clauses 115-124, further comprising, for each of the three versions of the 3D visual content: obtaining a plurality of sensor signals from a plurality of sensors; and determining a respective response parameter based on the plurality of sensor signals, wherein the respective response parameter includes one or more of: an eye blinking rate, a gaze direction, a fixation duration, a stress level, a focus level, a response time, a response accuracy level, and a micro expression type.

Clause 126. The method of any of Clauses 115-125, further comprising: providing respective response parameters of the three versions of the 3D visual content to a generative artificial intelligence (AI) model; and generating a message summarizing the respective response parameters of the three versions of the 3D visual content using the generative AI model.

Clause 127. A method for preparing an eyewear, comprising: at an electronic device including one or more processors, memory storing instructions, and a head-mounted display (HMD): executing a visual assessment application, including displaying a user interface to create a 3D virtual environment; obtaining a comprehensive prescription for an eyewear having a plurality of lens portions, each lens portion corresponding to one or more respective regions of a field of view and having a respective prescription parameter; obtaining 3D visual content for display on the user interface; and generating a multifocal prescription, including iteratively: rendering the 3D visual content based on the comprehensive prescription; and simplifying the comprehensive prescription, until an eyewear fitting condition is satisfied.

Clause 128. The method of Clause 127, iteratively simplifying the comprehensive prescription further comprising, successively implementing at least one set of operations of: adjusting a bifocal filter and rendering the 3D visual content based on the bifocal filter; adjusting a trifocal filter and rendering the 3D visual content based on the trifocal filter; and adjusting a progressive filter and rendering the 3D visual content based on the progressive filter.

Clause 129. An interactive virtual-reality method for performing a virtual vision test and displaying media, as discussed in any of Clauses 1-128.

Clause 130. A non-transitory computer readable storage medium, storing one or more programs for execution by one or more processors of a computer system, the one or more programs including instructions for implementing a method in any of Clauses 1-128.

Clause 131. An electronic device, comprising: one or more processors; and memory for storing one or more programs for execution by the one or more processors, the one or more programs including instructions for implementing a method in any of Clauses 1-128.

In some embodiments, any of the above clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

### FURTHER CONSIDERATIONS

As used herein, the word module refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase at least one of preceding a series of items, with the term and to separate any of the items, modifies the list as a whole, rather than each member of the list (i.e., each item). The phrase at least one of does not require selection of at least one of each item listed; rather, the phrase allows a meaning that includes at least one of any one of the items, and/or at least one of any combination of the items, and/or at least one of each of the items. By way of example, the phrases at least one of A, B, and C or at least one of A, B, or C each refer to only A, only B, or only C; any combination of A, B, and C; and/or at least one of each of A, B, and C.

Terms such as top, bottom, front, rear and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term include, have, or the like is used in the description or the claims, such term is intended to be inclusive in a manner similar to the term comprise as comprise is interpreted when employed as a transitional word in a claim.

As used herein, the term about is relative to the actual value stated, as will be appreciated by those of skill in the art, and allows for approximations, inaccuracies and limits of measurement under the relevant circumstances. In one or more aspects, the terms about, substantially, and approximately may provide an industry-accepted tolerance for their corresponding terms and/or relativity between items.

As used herein, the term comprising indicates the presence of the specified integer(s), but allows for the possibility of other integers, unspecified. This term does not imply any particular proportion of the specified integers. Variations of the word comprising, such as comprise and comprises, have correspondingly similar meanings.

The word exemplary is used herein to mean serving as an example, instance, or illustration. Any embodiment described herein as exemplary is not necessarily to be construed as preferred or advantageous over other embodiments.

A reference to an element in the singular is not intended to mean one and only one unless specifically stated, but rather one or more. Pronouns in the masculine (e.g., his) include the feminine and neuter gender (e.g., her and its) and vice versa. The term some refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope. In addition, it is not necessary for a device or method to address every problem that is solvable (or possess every advantage that is achievable) by different embodiments of the disclosure in order to be encompassed within the scope of the disclosure. The use herein of can and derivatives thereof shall be understood in the sense of possibly or optionally as opposed to an affirmative capability.

What is claimed is:

1. A method of implementing a vision test, comprising:
at an electronic device having a head-mounted display (HMD), one or more sensors, one or more processors, and memory:
executing a visual assessment application, including displaying a user interface to create a 3D virtual environment;
displaying visual content continuously for an extended duration of time in the 3D virtual environment, wherein the visual content is displayed with predefined display parameters associated with contact lens fitting;
obtaining a stream of sensor data measured by the one or more sensors;
applying at least a contact lens fitting model to generate a contact lens fitting profile for a user associated with the electronic device based on the stream of sensor data.

2. The method of claim 1, wherein the contact lens fitting profile includes at least one of: a fitting level of contact lenses worn by the user, one or more potential eye conditions and one or more associated occurrence probabilities, a suggested prescription adjustment, and one or more recommendations of contact lens types.

3. The method of claim 2, wherein the one or more potential eye conditions include a subset of eye redness, burning and itchiness, eye discharge, grittiness, light sensitivity, blurry vision, and dry eye.

4. The method of claim 1, further comprising:
determining a plurality of sequential user responses to the visual content based on the stream of sensor data; and
applying the contact lens fitting model to generate the contact lens fitting profile.

5. The method of claim 4, wherein the plurality of sequential user responses include one or more of: an eye blinking rate, a gaze direction, a fixation duration, a stress level, a focus level, an eye dryness level, an eye redness level, a fatigue level, a response time, a response accuracy level, and a micro expression type.

6. The method of claim 1, wherein the predefined display parameters include a plurality of corner display parameters each of which is substantially close to a respective display parameter limit, and the contact lens fitting profile is generated under a stressed display condition.

7. The method of claim 1, wherein the contact lens fitting profile includes a light sensitivity level, the method further comprising:
adjusting a color scheme, a contrast level, or a brightness level of the HMD

8. The method of claim 1, wherein the one or more sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

9. The method of claim 1, wherein the stream of sensor data are captured according to a temporal window, and each of the one or more sensors has a respective sampling rate and provides a subset of sensor data based on the respective sampling rate, and wherein the temporal window moves along a time axis.

10. The method of claim 9, further comprising:
for each of the one or more sensors, applying a sensor feature extraction model to process the subset of sensor data and generate a respective sensor feature vector; and
applying a response monitoring model to process respective sensor feature vectors of the one or more sensors and generate a respective sequential user response corresponding to the temporal window.

11. The method of claim 1, wherein the contact lens fitting profile is tracked during an extended duration of time to monitor an eye health condition associated with contact lens wearing.

12. A non-transitory computer readable storage medium, storing one or more programs for execution by one or more processors of an electronic device having an HMD and one or more sensors, the one or more programs including instructions for:
executing a visual assessment application, including displaying a user interface to create a 3D virtual environment;
displaying visual content continuously for an extended duration of time in the 3D virtual environment, wherein the visual content is displayed with predefined display parameters associated with contact lens fitting;
obtaining a stream of sensor data measured by the one or more sensors; and
applying at least a contact lens fitting model to generate a contact lens fitting profile for a user associated with the electronic device based on the stream of sensor data.

13. The non-transitory computer readable storage medium of claim 12, wherein the contact lens fitting profile includes at least one of: a fitting level of contact lenses worn by the user, one or more potential eye conditions and one or more associated occurrence probabilities, a suggested prescription adjustment, and one or more recommendations of contact lens types.

14. The non-transitory computer readable storage medium of claim 13, wherein the one or more potential eye conditions include a subset of eye redness, burning and itchiness, eye discharge, grittiness, light sensitivity, blurry vision, and dry eye.

15. The non-transitory computer readable storage medium of claim 12, the one or more programs further comprising instructions for:
determining a plurality of sequential user responses to the visual content based on the stream of sensor data; and
applying the contact lens fitting model to generate the contact lens fitting profile.

16. The non-transitory computer readable storage medium of claim 15, wherein the plurality of sequential user responses include one or more of: an eye blinking rate, a gaze direction, a fixation duration, a stress level, a focus level, an eye dryness level, an eye redness level, a fatigue level, a response time, a response accuracy level, and a micro expression type.

17. An electronic device, comprising:
an HMD;
one or more sensors;
one or more processors; and
memory for storing one or more programs for execution by the one or more processors, the one or more programs including instructions for:
executing a visual assessment application, including displaying a user interface to create a 3D virtual environment;
displaying visual content continuously for an extended duration of time in the 3D virtual environment, wherein the visual content is displayed with predefined display parameters associated with contact lens fitting;
obtaining a stream of sensor data measured by the one or more sensors; and
applying at least a contact lens fitting model to generate a contact lens fitting profile for a user associated with the electronic device based on the stream of sensor data.

18. The electronic device of claim 17, wherein the predefined display parameters include a plurality of corner display parameters each of which is substantially close to a respective display parameter limit, and the contact lens fitting profile is generated under a stressed display condition.

19. The electronic device of claim 17, wherein the contact lens fitting profile includes a light sensitivity level, the one or more programs further comprising instructions for:
adjusting a color scheme, a contrast level, or a brightness level of the HMD

20. The electronic device of claim 17, wherein the one or more sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.
